(19) United States
(12) Patent Application Publication
(10) Pub. No.: US 2024/0285571 A1
(43) Pub. Date: Aug. 29, 2024

(54) METHODS OF TREATING BONE INFECTIONS

(71) Applicant: MICROBION CORPORATION, Bozeman, MT (US)

(72) Inventor: Brett Hugh James BAKER, Bozeman, MT (US)

(21) Appl. No.: 18/247,637

(22) PCT Filed: Oct. 1, 2021
(86) PCT No.: PCT/US2021/053194

§ 371 Date: Mar. 31, 2023

Related U.S. Application Data
(60) Provisional application No. 63/086,438, filed on Oct. 1, 2020.

(51) Int. Cl. A61K 31/29; A61K 45/06

(52) U.S. Cl. CPC A61K 31/29; A61K 45/06; A61L 2300/404

(57) ABSTRACT

The present disclosure relates to bismuth-thiol compounds such as bismuth-1,2-dithiane (BisEDT) for use in treating, preventing and/or reducing the effects of osteomyelitis in a subject in need thereof.

### CROSS REFERENCE TO RELATED APPLICATIONS

This application claims the benefit of U.S. Provisional Application No. 63/086,438, filed on Oct. 1, 2020, which is incorporated herein by reference in its entirety.

### BACKGROUND

Osteomyelitis is associated with significant morbidity and mortality. In particular, postoperative orthopaedic infections, particularly antibiotic-resistant infections, present a serious clinical challenge to surgeons and other treating physicians because these infections involve foreign materials (e.g., stabilizing orthopaedic hardware) which are frequently associated with persistent microbial biofilms. Surgical intervention, including irrigation, debridement and potential replacement of orthopaedic hardware, combined with a prolonged course of systemic antibiotics, is the standard of care for postoperative infections. However, outcomes associated with these infections are often poor, including chronic/recurrent infections, repeated hospitalizations, repeated surgeries, multiple courses of systemic antibiotic treatment, loss of function, disability, amputation and death.

Current antimicrobial treatments for osteomyelitis, have generally shown inadequate efficacy due to the limited access of systemically administered antibiotics to sites where causative bacteria can reside, including as biofilms on bone surfaces. In fact, it has been found that treatment of heavily contaminated or infected wounds with systemic antibiotics often has practical and potential value only if a therapeutic blood level or, more importantly, tissue level is achieved within the first 4 hours after wounding, i.e., before bacterial biofilm adherence to bone and geographically related tissues, and orthopaedic hardware surfaces. The competition between bacteria and host tissues with respect to speed of colonization of foreign objects such as orthopaedic medical devices is a key factor. Bacteria are able to adhere to foreign, implanted objects almost immediately, facilitating rapid formation of microbial biofilms which drastically increase the resistance of wound-associated bacteria and contribute significantly to persistence and virulence of the infection. Such biofilms can serve as quiescent reservoirs of adherent, antibiotic-tolerant or antibiotic-resistant bacteria within the wound, or as foci of active infections, which helps to explain the chronic and/or recurrent nature of many device-related infections. In addition, biofilms are hypermutable, increasing the likelihood of developing genetically-based antibiotic resistance. Finally, biofilms serve as environments in which bacteria can exchange genetic material encoding antibiotic resistance genes. Recent studies have shown that the most highly multidrug resistant (MDR) bacteria are also the strongest biofilm-forming bacteria, and similarly, that invasive MDR bacteria are usually biofilm-forming bacteria.

Development of local therapies that eradicate established biofilms or prevent the formation of biofilms (as a new target) in addition to overcoming the broad spectrum of antibiotic-resistant bacterial pathogens (including anaerobes) would be an innovative new clinical strategy, particularly considering the current shortcomings of systemically administered antibiotics for treatment of wound infections. However current approaches, such as using polymethylmethacrylate beads as non-biodegradable carrier systems to deliver antibiotics to orthopaedic infections, require surgical removal upon completion of drug release. They also tend to release antibiotics in an initial burst pattern that quickly depletes the bulk of the drug from the carrier beads, followed by a slow release at lower concentrations that may not be adequate to control infection and may foster development of resistance. These concerns limit the usefulness of this approach in the majority of bone and joint infections.

Thus, the discovery and development of more effective and physiologically targeted local delivery systems are needed.

### SUMMARY

To address the aforementioned issues, as well as others, the present disclosure provides compositions and methods useful for treating osteomyelitis and associated conditions.

In some aspects, the present disclosure provides a method of treating osteomyelitis, bone infection, or infection near a bone or orthopaedic device inserted in a subject, comprising administering to the subject in need thereof a therapeutically effective amount of a bismuth-thiol (BT) composition that comprises BisEDT suspended therein, wherein the BT composition is topically applied near or on a site of an infected bone of said subject.

In some aspects, the present disclosure provides a method of preventing osteomyelitis bone infection, or infection near a bone or orthopaedic device inserted in a subject, comprising administering to a subject in need thereof a therapeutically effective amount of a bismuth-thiol (BT) composition that comprises BisEDT suspended therein, wherein the BT composition is topically applied to a site susceptible to a bone infection.

In some aspects, the present disclosure provides a method of preventing osteomyelitis, bone infection, or infection near a bone or orthopaedic device inserted in a subject, comprising administering to a subject in need thereof a therapeutically effective amount of a bismuth-thiol (BT) composition that comprises BisEDT suspended therein, wherein the BT composition is topically applied to an infected site capable of causing a bone infection, e.g., an open wound, an open fracture, puncture, etc.

In some embodiments, the composition is a suspension of microparticles having a volumetric mean diameter (VMD) from about 0.4 μm to about 5 μm. In some embodiments, the composition is a suspension of microparticles having a volumetric mean diameter (VMD) from about 0.6 μm to about 3 μm. In some embodiments, the composition is a suspension of microparticles having a volumetric mean diameter (VMD) from about 1 μm to about 2 μm.

In some embodiments, the composition comprises BisEDT at a concentration of 0.025 mg/mL or greater, about 3% methylcellulose, about 0.5% Tween 80, about 10 mM sodium chloride, and about 10 mM sodium phosphate at about pH 7.4. In some embodiment, the BisEDT concentration is from about 0.025 mg/mL to about 0.25 mg/mL.

In some aspects, the present disclosure provides a method for treating osteomyelitis, bone infection, or infection near a bone or orthopaedic device inserted in a subject, comprising administering to a subject in need thereof a therapeutically effective amount of a composition, wherein the composition is a suspension of microparticles having a volumetric mean diameter (VMD) from about 0.4 μm to about 5 μm, and comprising BisEDT at a concentration of 0.025 mg/mL or greater, about 3% methylcellulose, about 0.5% Tween 80®, about 10 mM sodium chloride, and about 10 mM sodium phosphate at about pH 7.4, and wherein the composition is applied directly on an infected bone of said subject

In some aspects, the present disclosure provides a method for treating an osteosynthesis-associated infection, comprising intraoperatively administering to a subject in need thereof a therapeutically effective amount of a composition, wherein the composition is a suspension of microparticles having a volumetric mean diameter (VMD) from about 0.4 μm to about 5 μm, and comprising BisEDT at a concentration of 0.025 mg/mL or greater, about 3% methylcellulose, about 0.5% Tween 80®, about 10 mM sodium chloride, and about 10 mM sodium phosphate, and wherein the composition is applied either (a) directly to structures within infected osteosynthesis sites during revision surgery with or without hardware removal; or (b) directly to the immediate soft tissue and bone in subjects with chronic or acute-on-chronic osteomyelitis of the long bone extremities or residual amputated limbs.

In some embodiments, the infection is a bacterial infection. In some embodiments, the infection is a fungal infection. In some embodiments, the infection is caused by one of more of the following pathogens Corynebacterium jeikeium, Corynebacterium, non-speciated, Actinomyces turicensis, Corynebacterium amycolatum, Corynebacterium resistens, Corynebacterium simulans, Dermabacter hominis, Staphylococcus epidermidis, Staphylococcus aureus, MRSA, Staphylococcus aureus, MSSA, Staphylococcus lugdunensis, Enterococcus faecalis, Granulicatella, non-speciated, Staphylococcus arlettae, Staphylococcus haemolyticus, Staphylococcus hominis, Staphylococcus pasteuri, Staphylococcus warneri, Streptococcus oralis, Enterobacter cloacae, Serratia marcescens, Escherichia coli, Klebsiella oxytoca, Pseudomonas aeruginosa, Stenotrophomonas maltophilia, Cutibacterium (Propionibacterium) acnes, Finegoldia magna, Anaerococcus murdochii, Anaerococcus, non-speciated, Clostridium sphenoides, Peptoniphilus gorbachii, Prevotella bergensis, and/or Candida parapsilosis. In some embodiments, the infection is caused by Staphylococcus aureus. In some embodiments, the Staphylococcus aureus is methicillin-susceptible Staphylococcus aureus (MSSA). In some embodiments, the Staphylococcus aureus is methicillin-resistant Staphylococcus aureus (MRSA).

In some aspects, the present disclosure provides a method for treating and/or preventing a fungal infection as a result of an orthopaedic procedure, comprising administering to the subject in need thereof a therapeutically effective amount of a composition, wherein the composition is a suspension of microparticles having a volumetric mean diameter (VMD) from about 0.4 μm to about 5 μm, and comprising BisEDT at a concentration 0.025 mg/mL or greater, about 3% methylcellulose, about 0.5% Tween 80, about 10 mM sodium chloride, and about 10 mM sodium phosphate at about pH 7.4, and wherein the composition it applied directly on an infected site of said subject.

In some embodiments, the composition remains active on the site of direct application after 1 week, 2 weeks, 3 weeks, 1 month, 2 months, 3, months, 4 months, or 5 months. In some embodiments, the composition remains active on the site of direct application after 1 week, 1 month, or 2 months.

The present disclosure also provides a pharmaceutical composition comprising bismuth-thiol (BT) composition that comprises BisEDT suspended therein and at least one antimicrobial agent, wherein the BT composition comprises a plurality of microparticles, wherein the D90 of said microparticles is less than or equal to 2 μm.

### BRIEF DESCRIPTION OF THE DRAWINGS

FIG. 1 shows the activity of BT compounds against biofilms grown from MR14, which is a multidrug-resistant isolate of Pseudomonas aeruginosa.

FIG. 2 shows the activity of BT compounds against biofilms grown from AG14, which is an aminoglycoside-resistant isolate of Pseudomonas aeruginosa.

FIG. 3 shows the activity of BT compounds against biofilms grown from AU197, which is a isolate of Burkholderia cenocepacia.

FIG. 4 shows the activity of BT compounds against biofilms grown from AMT0130-8, which represents an isolate of the clinically relevant Mycobacterium abscessus complex (MABSC).

FIG. 5 shows the activity of BT compounds against biofilms grown from AMT0089-5, which is a macrolide-resistant, amikacin-resistant MABSC.

FIG. 6 shows the activity of BT compounds against biofilms grown from ATCC-19977, which is M. abscessus (macrolide-resistant; inducible).

FIG. 7 shows the activity of BT compounds against biofilms grown from MABSC isolate.

FIG. 8 shows the activity of BT compounds against biofilms grown from Achromobacter spp.

FIG. 9 shows the activity of BT compounds against biofilms grown from Stenotrophomonas maltophilia.

FIG. 10 shows the activity of BT compounds against biofilms grown from E. coli.

FIG. 11 is a summary of the clinical trial study evaluating the efficacy of MBN-101 (aqueous liquid suspension comprising BisEDT) in treating infected osteosynthesis sites during revision surgery or sites of chronic or acute-on-chronic osteomyelitis.

FIG. 12 provides a chart for subject disposition based on the clinical trial design for the study evaluating the efficacy of MBN-101.

### DEFINITIONS

Unless defined otherwise, all technical and scientific terms used herein have the meaning commonly understood by a person skilled in the art of the present disclosure. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the verb “comprise” as is used in this description and in the claims and its conjugations are used in its non-limiting sense to mean that items following the word are included, but items not specifically mentioned are not excluded. The present disclosure may suitably “comprise”, “consist of”, or “consist essentially of”, the steps, elements, and/or reagents described in the claims.

Unless specifically stated or obvious from context, as used herein, the term “or” is understood to be inclusive. Unless specifically stated or obvious from context, as used herein, the terms “a”, “an”, and “the” are understood to be singular or plural.

Throughout the present specification, the terms “about” and/or “approximately” may be used in conjunction with numerical values and/or rages. The term “about” is understood to mean those values near to a recited value. Furthermore, the phrases “less than about[a value]” or “greater than about[a value]” should be understood in view of the definition of the term “about” provided herein. The terms “about” and “approximately” may be used interchangeably.

An “alkyl” group or “alkane” is a straight chained or branched non-aromatic hydrocarbon which is completely saturated. Typically, a straight chained or branched alkyl group has from 1 to about 20 carbon atoms, e.g. from 1 to about 10 unless otherwise defined. Examples of straight chained and branched alkyl groups include methyl, ethyl, n-propyl, iso-propyl, n-butyl, sec-butyl, tert-butyl, pentyl, hexyl, pentyl and octyl. A C₁-C₆ straight chained or branched alkyl group is also referred to as a “lower alkyl” group.

Moreover, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate. For instance, the substituents of a substituted alkyl can include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y}” when used in conjunction with a chemical moiety, such as, acyl, acyloxy, alkyl, alkenyl, alkynyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylthio”, as used herein, refers to a thiol group substituted with an alkyl group and can be represented by the general formula alkylS—.

The terms “amine” and “amino” are art-recognized and refer to both unsubstituted and substituted amines and salts thereof, e.g., a moiety that can be represented by

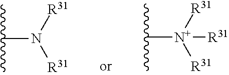

wherein each R³¹ independently represents a hydrogen or a hydrocarbyl group, or two R³¹ are taken together with the N atom to which they are attached complete a heterocycle having from 4 to 8 atoms in the ring structure. The term “aminoalkyl”, as used herein, refers to an alkyl group substituted with an amino group.

The term “aryl” as used herein include substituted or unsubstituted single-ring aromatic groups in which each atom of the ring is carbon. In some embodiments, the ring is a 5- to 7-membered ring, e.g. a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “bismuth” refers to the 83^{rd} element of the periodic table, or atoms or ions thereof. Bismuth can occur in the metallic state or in the ionized state, such as in the III or V oxidation state. Bismuth ions can form complexes with anions, either to make bismuth salts, or to form complex anions which are then further complexed with one or more additional cation(s). Bismuth can also form covalent bonds to other atoms, such as sulfur.

As disclosed herein, a “bismuth-thiol compound” or “BT compound” is a compound that has a bismuth atom covalently bound to one, two or three other sulfur atoms present on one or more thiol compounds. The term “thiol” refers to a carbon-containing compound, or fragment thereof, containing an —SH group and can be represented by the general formula R—SH. These thiol compounds include compounds with one, two, three or more S atoms. Thiol compounds can have other functionality, such as alkyl, hydroxyl, carbocyclyl, heterocyclyl, aryl, heteroaryl, amino, and other substituents. Thiol compounds having two or more S atoms can chelate the bismuth atom, such that two S atoms from the same molecule covalently bond with the bismuth atom. Exemplary bismuth-thiol compounds are shown below:

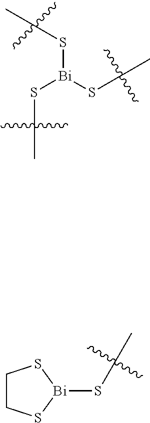

The terms “carbocycle”, and “carbocyclic”, as used herein, refers to a saturated or unsaturated ring in which each atom of the ring is carbon. The term carbocycle includes both aromatic carbocycles and non-aromatic carbocycles. Non-aromatic carbocycles include both cycloalkane rings, in which all carbon atoms are saturated, and cycloalkene rings, which contain at least one double bond.

The term “carbocycle” includes 5-7 membered monocyclic and 8-12 membered bicyclic rings. Each ring of a bicyclic carbocycle can be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle can be selected from saturated, unsaturated and aromatic rings. In an exemplary embodiment, an aromatic ring, e.g., phenyl, can be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” can be substituted at any one or more positions capable of bearing a hydrogen atom.

A “cycloalkyl” group is a cyclic hydrocarbon which is completely saturated. “Cycloalkyl” includes monocyclic and bicyclic rings. Typically, a monocyclic cycloalkyl group has from 3 to about 10 carbon atoms, more typically 3 to 8 carbon atoms unless otherwise defined. The second ring of a bicyclic cycloalkyl can be selected from saturated, unsaturated and aromatic rings. Cycloalkyl includes bicyclic molecules in which one, two or three or more atoms are shared between the two rings. The term “fused cycloalkyl” refers to a bicyclic cycloalkyl in which each of the rings shares two adjacent atoms with the other ring. The second ring of a fused bicyclic cycloalkyl can be selected from saturated, unsaturated and aromatic rings. A “cycloalkenyl” group is a cyclic hydrocarbon containing one or more double bonds.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The term “heteroalkyl”, as used herein, refers to a saturated or unsaturated chain of carbon atoms and at least one heteroatom, wherein no two heteroatoms are adjacent.

The terms “heteroaryl” and “hetaryl” include substituted or unsubstituted aromatic single ring structures, for example 5- to 7-membered rings, e.g. 5- to 6-membered rings, whose ring structures include at least one heteroatom, for example one to four heteroatoms, e.g. one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The terms “heterocyclyl”, “heterocycle”, and “heterocyclic” refer to substituted or unsubstituted non-aromatic ring structures, for example, 3- to 10-membered rings, more e.g. 3- to 7-membered rings, whose ring structures include at least one heteroatom, e.g. one to four heteroatoms, e.g. one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocyclylalkyl”, as used herein, refers to an alkyl group substituted with a heterocycle group.

The term “hydrocarbyl”, as used herein, refers to a group that is bonded through a carbon atom that does not have a ═O or ═S substituent, and typically has at least one carbon-hydrogen bond and a primarily carbon backbone, but can optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocyclyl, alkyl, alkenyl, alkynyl, and combinations thereof.

The term “lower” when used in conjunction with a chemical moiety, such as, acyl, acyloxy, alkyl, alkenyl, alkynyl, or alkoxy is meant to include groups where there are ten or fewer non-hydrogen atoms in the substituent, for example, six or fewer. A “lower alkyl”, for example, refers to an alkyl group that contains ten or fewer carbon atoms, e.g. six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, e.g. from 5 to 7.

The term “N-oxide” refers to a zwitterionic group containing a nitrogen atom in the +1 oxidation state bound to an oxygen atom in the −1 oxidation state. An non-limiting example of an N-oxide is pyridium N-oxide shown below. As used herein, the term “N-oxide” encompasses substituents of other groups having this functionality.

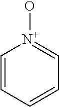

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen can have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that substituents can themselves be substituted, if appropriate. Unless specifically stated as “unsubstituted,” references to chemical moieties herein are understood to include substituted variants. For example, reference to an “aryl” group or moiety implicitly includes both substituted and unsubstituted variants.

The term “thioalkyl”, as used herein, refers to an alkyl group substituted with a thiol group. A “thiol compound” as discussed above can include a thioalkyl as a substituent on the compound structure. A thiol compound can have, for example, one, two, three or more thioalkyl groups.

The term “thioether”, as used herein, is equivalent to an ether, wherein the oxygen is replaced with a sulfur.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week.

“Coadministration” refers to the administration of the two agents in any manner in which the pharmacological effects of both agents are manifest in the patient at the same time. Thus, concomitant administration does not require that a single pharmaceutical composition, the same dosage form, or even the same route of administration be used for administration of both agents or that the two agents be administered at precisely the same time. However, in some situations, coadministration will be accomplished most conveniently by the same dosage form and the same route of administration, at substantially the same time.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more signs and symptoms of the disorder or condition relative to the untreated control sample.

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “managing” includes therapeutic treatments as defined above. Managing includes achieving a steady state level of infection as determined by known methods in the art. The steady state can include evaluation of one or more of the severity of the infection(s), the size and location of the infection(s), the number of different microbial pathogens present in the infection(s), the level of antibiotic tolerant or resistant microbial pathogens, the degree of response to treatment, such as with a BT composition disclosed herein, the degree of biofilm formation and reduction, and the side effects experienced by the subject. During management of an infection, the infection may fluctuate from increasing to lessening in severity, in the amount or extent of infection, amount of side effects experienced by the subject, or other subject outcome indicia. Over a period of time, such as days, month, or years, the degree of management of the infection can be determined by evaluation of the above factors to assess whether the clinical course of infection has improved, is bacteriostatic, or has worsened. In some embodiments, managing an infection include successful treatment of microbial pathogen(s) that are otherwise drug tolerant or drug resistant.

The term “lessen the severity” of infection(s) refers to an improvement in the clinical course of the infection on any measurable basis. Such basis can include measurable indices such as reducing the extent of infection(s), whether the infection(s) are considered acute, the number and identity of microbial pathogens causing the infection(s), the extent/spread/amount of microbial (e.g. bacterial and/or fungal) biofilms, and side effects experienced by the subject. In some embodiments, lessening the severity of an infection is determined by measuring an improvement in clinical signs and symptoms of infection. In some embodiments, lessening the severity involves halting a steady decline in outcome to achieve stabilized infection(s), resulting in the subject entering successful management of the infection(s). In other embodiments, lessening the severity can result in substantial to complete treatment of the infection(s).

In some embodiments, lessening the severity of infections and/or symptoms can relate to patient-reported outcomes (“PROs”). A PRO instrument is defined as any measure of a subject's health status that is elicited from the patient and determines how the patient “feels or functions with respect to his or her health condition.” PROs are particularly useful in reporting outcomes in DFI and whether the severity of symptoms has been reduced or lessened. Such symptoms can be observable events, behaviors, or feelings (e.g., ability to walk quickly, lack of appetite, expressions of anger), or unobservable outcomes that are known only to the patient (e.g., perceptions of pain, feelings of depression). In some embodiments, lessening the severity of infections and/or symptoms can be determined by physician assessments commonly known in the art, for example by an 8 item wound score.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of an infection.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

A “response” to a method of treatment can include, among other things, a decrease in or amelioration of negative signs and symptoms, a decrease in the progression of an infection or symptoms thereof, an increase in beneficial symptoms or clinical outcomes, a lessening of side effects, stabilization of the infection, and partial or complete remedy of infection, partial or full wound closure, reduction in wound size, or complete or substantially complete re-epithelialization, among others.

“Antibiotic susceptibility or sensitivity” refers to whether a bacteria will be successfully treated by a given antibiotic. Similarly, “Antifungal susceptibility or sensitivity” refers to whether a fungi will be successfully treated by a given antibiotic. Testing for susceptibility can be performed by methods known in the art such as the Kirby-Bauer method, the Stokes method and Agar Broth dilution methods. The effectiveness of an antibiotic in killing the bacteria or preventing bacteria from multiplying can be observed as areas of reduced or stable amount, respectively, of bacterial growth on a medium such as a wafer, agar, or broth culture.

“Antimicrobial tolerance” refers to the ability of a microbe, such as bacteria or fungi, to naturally resist being killed by antibiotics. It is not caused by mutant microbes but rather by microbial cells that exist in a transient, dormant, non-dividing state. Antibiotic or drug tolerance is caused by a small subpopulation of microbial cells termed persisters. Persisters are not mutants, but rather are dormant cells that can survive the antimicrobial treatments that kill the majority of their genetically identical siblings. Persister cells have entered a non- or extremely slow-growing physiological state which makes them insensitive (refractory or tolerant) to the action of antimicrobial drugs. Similarly, “antibiotic tolerance” refers to the ability of a bacteria to naturally resist being killed by antibiotics and “antifungal tolerance” refers to the ability of a fungi to naturally resist being killed by antibiotics.

“Antimicrobial resistance” refers to the ability of a microbe to resist the effects of medication that once could successfully treat the microbe. Microbes resistant to multiple antimicrobials are called multidrug resistant (MDR). Resistance arises through one of three mechanisms: natural resistance in certain types of bacteria, genetic mutation, or by one species acquiring resistance from another. Mutations can lead to drug inactivation, alteration of the drugs binding site, alteration of metabolic pathways and decreasing drug permeability.

As used herein, the term “in combination” or “in further combination” or “further in combination” refers to the use of an additional prophylactic and/or therapeutic agent as well as a BT composition of the present disclosure. The use of the term “in combination” does not restrict the order in which prophylactic and/or therapeutic agents are administered to a subject. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent (different from the first prophylactic or therapeutic agent) to a subject.

As used herein, the terms “prophylactic agent” and “prophylactic agents” refer to an agent, such as a BT composition of the present disclosure, which can be used in the prevention, management, or control of one or more signs and symptoms of a disease or disorder, in particular, a disease or disorder associated with a microbial (e.g. bacterial and/or fungal) infection, such as diabetic foot infection.

As used herein, the terms “antibacterial activity”, “antifungal activity” and “antimicrobial activity”, with reference to a BT composition of the present disclosure, refers to the ability to kill and/or inhibit the growth or reproduction of a particular microorganism. In certain embodiments, antibacterial or antimicrobial activity is assessed by culturing bacteria, e.g., Gram-positive bacteria (e.g., S. aureus), Gram-negative bacteria (e.g., A. baumannii, E. coli, and/or P. aeruginosa) or bacteria not classified as either Gram-positive or Gram-negative, or fungi according to standard techniques (e.g., in liquid culture or on agar plates), contacting the culture with a BT composition of the present disclosure and monitoring cell growth after said contacting. For example, in a liquid culture, bacteria may be grown to an optical density (“OD”) representative of a mid-point in exponential growth of the culture; the culture is exposed to one or more concentrations of one or more BT compounds of the present disclosure, or variants thereof, and the OD is monitored relative to a control culture. Decreased OD relative to a control culture is representative of antibacterial activity (e.g., exhibits lytic killing activity). Similarly, bacterial colonies can be allowed to form on an agar plate, the plate exposed to a BT composition of the present disclosure, or variants thereof, and subsequent growth of the colonies evaluated related to control plates. Decreased size of colonies, or decreased total numbers of colonies, indicate antibacterial activity.

“Biofilm” refers any syntrophic consortium of microorganisms in which cells stick to each other and often also to a surface. These adherent cells become embedded within a slimy extracellular matrix that is composed of extracellular polymeric substances (EPS). Upon formation of biofilms, microbial resistance to antibiotics is up to 1000 times greater compared to that of planktonic bacteria. Bacterial aggregates are clusters of laterally aligned cells can initiate biofilm development, which has a more complex and denser 3-D structure. In some embodiments, the biofilm may comprise one or more species of bacteria (e.g., Pseudomonas aeruginosa and Staphylococcus aureus) and/or one or more different phyla (e.g., bacteria, virus and fungi).

The term “infection” is used herein in its broadest sense and refers to any infection, such as viral infection or caused by a microorganism bacterial infection, fungal infection or parasitic infection (e.g. protozoa, amoeba or helminths). Examples of such infections can be found in a number of well-known texts such as “Medical Microbiology” (Greenwood, D., Slack, R, Peutherer, J., Churchill Livingstone Press, 2002); “Mims' Pathogenesis of Infectious Disease” (Mims, C., Nash, A., Stephen, J., Academic Press, 2000); “Fields” Virology. (Fields, B N, Knipe D M, Howley, P M, Lippincott Williams and Wilkins, 2001); and “The Sanford Guide To Antimicrobial Therapy,” 26th Edition, JP Sanford et al. (Antimicrobial Therapy, Inc., 1996), which is incorporated by reference herein. The presence of infection in e.g. a diabetic foot wound is defined by clinical signs and symptoms of infection or inflammation, not by the culture of microorganisms, which are always present. However, immediately following resolution of clinical signs and symptoms of a wound infection, most patients will still have the underlying ulcer (e.g. diabetic foot ulcer), which requires continued treatment to facilitate complete wound closure. Of note, however, is that many wound specialists believe that in addition to the clinically defined state of infection, a less clinically apparent pathological state, known as “critical colonization” exists. In this state, a wound may be delayed or arrested in wound healing due to the subclinical presence of a high level of bacteria. This critical colonization, sometimes referred to as a high ‘wound bioburden’, is often polymicrobial and associated with biofilm-producing bacteria; it has been shown to induce, or prolong, the active inflammatory phase of repair, thus preventing a normal wound healing process. The bacterial cells that comprise such biofilms are difficult to recognize because they often exist in a viable, but nonculturable (VBNC), state (Pasquaroli 2013), yet they are adherent to surfaces and are typically more tolerant and resistant than their planktonic counterparts to antibiotics and antiseptics (Costerton 1999, Nguyen 2011). The term “infection” therefore contemplates the clinically defined state of infection as well as “critical colonization.”

The term “wound closure” can encompass healing of a wound wherein sides of the wound are rejoined to form a continuous barrier (e.g., intact skin). In another embodiment, the compositions and methods provided herein promote tissue regeneration. In another embodiment, the compositions and methods provided herein limit scarring of tissues such as glia, tendons, eye tissue, ligament or skin. In some embodiments, “wound closure” refers to complete or substantially complete re-epithelialization. In some embodiments, “wound closure” occurs via secondary intention.

It is to be understood that the term “wound healing” can encompass a regenerative process with the induction of a temporal and spatial healing program comprising wound closure and the processes involved in wound closure. The term “wound healing” can also encompass the processes of granulation, neovascularization, fibroblast, endothelial and epithelial cell migration, extracellular matrix deposition, re-epithelialization, and remodeling. In some embodiments, “wound healing” refers to a wound remaining closed for a sufficient period of time after the initial wound closure (e.g. one day, two days, three days, four days, five days, six days, one week, two weeks, three weeks, or one month). In some embodiments, “wound healing” refers to a wound remaining closed for two weeks after the initial wound closure.

It will be appreciated by a skilled artisan that the term “granulation” can encompass the process whereby small, red, grainlike prominences form on a raw surface (that of wounds or ulcers) as healing agents. Granulation may also include the formation of granulation tissue over the wound. “Granulation tissue” refers to the newly growing tissue material at a wound site formed to heal the wound. The tissue is perfused, fibrous connective tissue including a variety of cell types. The tissue will grow generally from the base of the wound to gradually fill the entire wound space.

It will be appreciated by a skilled artisan that the term “neovascularization” can encompass the new growth of blood vessels with the result that the oxygen and nutrient supply is improved. Similarly, it will be appreciated by the skilled artisan that the term “angiogenesis” may encompass the vascularization process involving the development of new capillary blood vessels. It will also be appreciated that the term “cell migration” refers to the movement of cells (e.g., fibroblast, endothelial, epithelial, etc.) to the wound site.

It is to be understood that the term “extracellular matrix deposition” can encompass the secretion by cells of fibrous elements (e.g., collagen, elastin, reticulin), link proteins (e.g., fibronectin, laminin), and space filling molecules (e.g., glycosaminoglycans). It will be appreciated by the skilled artisan that the term “type I collagen” can encompass the most abundant collagen, which forms large well-organized fibrils having high tensile strength.

It will be appreciated by a skilled artisan that the term “re-epithelialization” can encompass the reformation of epithelium over a denuded surface (e.g., wound).

The term “remodeling” refers to the replacement of and/or devascularization of granulation tissue.

As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

As used herein, “D90” refers to the 90% value of particle diameter (i.e. the microparticle). For example if D90=1 μm, 90% of the particles are smaller than 1 μm. Similarly, “D80” refers to the 80% value of particle diameter (i.e. the microparticle), “D70” refers to the 70% value of particle diameter (i.e. the microparticle), “D60” refers to the 60% value of particle diameter (i.e. the microparticle), “D50” refers to the 50% value of particle diameter (i.e. the microparticle), “D40” refers to the 40% value of particle diameter (i.e. the microparticle), “D30” refers to the 30% value of particle diameter (i.e. the microparticle), “D20” refers to the 20% value of particle diameter (i.e. the microparticle), “D10” refers to the 10% value of particle diameter (i.e. the microparticle).

As used herein, “osteomyelitis” refers to the inflammation of bone or bone marrow, or infection of the bone or bone marrow, such as a fungal, or bacterial infection.

As used herein, MBN-101 refers to a composition comprising BisEDT.

### DETAILED DESCRIPTION

### Pharmaceutical Compositions

In some embodiments, the present disclosure provides a pharmaceutical composition comprising a bismuth-thiol (BT) compound.

The compositions of the present disclosure can be utilized to treat a subject in need thereof. In certain embodiments, the subject is a mammal such as a human, or a non-human mammal. When administered to subject, such as a human, the composition or the compound is preferably administered as a pharmaceutical composition comprising, for example, a compound of the disclosure (i.e., a BT compound such as BisEDT) and a pharmaceutically acceptable carrier. Pharmaceutically acceptable carriers are well known in the art and include, for example, aqueous solutions such as water, physiologically buffered saline, physiologically buffered phosphate, or other solvents or vehicles such as glycols, glycerol, oils such as olive oil, or injectable organic esters. In some embodiments, when such pharmaceutical compositions are for human administration, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as lyophile for reconstitution, powder, solution, syrup, injection or the like. The composition can also be present in a suspension or solution suitable for topical administration.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose, or dextrans; antioxidants, such as ascorbic acid or glutathione; chelating agents; low molecular weight proteins; salts; or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of a subject without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the subject. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, methyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols and sugar alcohols, such as glycerin, sorbitol, mannitol, xylitol, erythritol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances, including salts such as sodium chloride, employed in pharmaceutical formulations.

The formulations can conveniently be presented in unit dosage form and can be prepared by any methods well known in the art of pharmacy. The amount of active ingredient which can be combined with a carrier material to produce a single dosage form will vary depending upon the subject being treated, the particular mode of administration. The amount of active ingredient that can be combined with a carrier material to produce a single dosage form will generally be that amount of the compound which produces a therapeutic effect.

In some embodiments, the BT composition is a powder, spray, ointment, paste, cream, lotion, suspension, solution, patch, suspension or gel. In some embodiments, the BT composition is a solution. In some embodiments, the BT composition is a suspension. In some embodiments, the suspension comprises a plurality of microparticles as defined herein.

The BT composition can comprise any suitable concentration of bismuth-thiol compound. In some embodiments, the BT composition is administered as a dosage from about 0.025 mg/mL to about 15 mg/mL, from about 0.04 mg/mL to about 15 mg/mL, from about 0.06 mg/mL to about 15 mg/mL, from about 0.06 mg/mL to about 10.0 mg/mL, from about 0.5 mg/mL to about 10.0 mg/mL, from about 1.0 mg/mL to about 100 mg/mL, from about 25 mg/mL to about 100 mg/mL, from about 50 mg/mL to about 100 mg/mL, from about 0.8 mg/mL to about 15 mg/mL, from about 1 mg/mL to about 10 mg/mL, from 2.5 mg/mL to about 10 mg/mL, from about 4 mg/mL to about 10 mg/mL, from about 5 mg/mL to about 10 mg/mL, from about 6 mg/mL to about 10 mg/mL, 0.6 mg/mL to about 6 mg/mL, from about 4 mg/mL to about 15 mg/mL, from about 6 mg/mL to about 15 mg/mL, from about 50 μg/mL to about 750 μg/mL, from about 75 μg/mL to about 500 μg/mL, from about 100 μg/mL to about 250 μg/mL, from about 100 μg/mL to about 150 μg/mL, or from about 75 μg/mL to about 150 μg/mL; and/or the total amount of the BT composition administered to the lungs is from about 0.25 mg to about 15 mg, from about 0.4 mg to about 15 mg, from about 0.6 mg to about 15 mg, from about 0.8 mg to about 15 mg, from about 1 mg to about 10 mg, from 2.5 mg to about 10 mg, from about 4 mg to about 10 mg, from about 5 mg to about 10 mg, from about 6 mg to about 10 mg, 0.6 mg to about 6 mg, from about 4 mg to about 15 mg, from about 6 mg to about 15 mg, from about 50 μg to about 750 μg, from about 75 μg to about 500 μg, from about 100 μg to about 250 μg, from about 100 μg to about 150 μg, or from about 75 μg to about 150 μg. In some embodiments, the BT composition is administered as a dosage from about 0.025 mg/mL to about 10 mg/mL. In some embodiments, the BT composition is administered as a dosage from about 0.025 mg/mL to about 5 mg/mL. In some embodiments, the BT composition is administered as a dosage from about 0.025 mg/mL to about 5 mg/mL. In some embodiments, the BT composition is administered as a dosage from about 0.025 mg/mL to about 1.0 mg/mL. In some embodiments, the BT composition is administered as a dosage from about 0.025 mg/mL to about 0.5 mg/mL. In some embodiments, the BT composition is administered as a dosage from about 0.025 mg/mL to about 0.25 mg/mL. In certain embodiments, the BT composition is administered as a dosage from about 0.6 mg/mL to about 6 mg/mL.

In some embodiments, the BT composition is administered three times per day, two times per day, once daily, every other day, once every three days, once every week, once every other week, once monthly, to once every other month. In certain embodiments, the BT composition is administered once daily. In certain embodiments, the BT composition is administered once weekly. In certain embodiments, the BT composition is administered once every other week. In some embodiments, the BT composition is administered chronically in a 4 week on/4 week off dosing schedule. In some embodiments, the BT composition is administered chronically, for example as part of a background therapy. As will be appreciated by a person having ordinary skill in the art, the administration frequency may depend on a number of factors including dose and administration route. For example, if the BT composition is administered via an topical administration, a low dose such as 100-1000 μg/mL may be administered once or twice daily; however, a high dose such as 2.5-10 mg/mL may be administered e.g. once or twice a week.

In some embodiments, the BT composition further comprises one or more carriers selected from animal and vegetable fats, oils, waxes, paraffins, starch, tragacanth, cellulose derivatives, polyethylene glycols, silicones, bentonites, silicic acid, polymers, talc, and zinc oxide. In some embodiments, the carrier is methylcellulose. In some embodiments, the carrier is poly(methyl methacrylate).

Compositions can also be formulated so as to provide slow or controlled release of the active ingredient therein using, for example, hydroxypropylmethyl cellulose in varying proportions to provide the desired release profile, other polymer matrices, liposomes and/or microspheres. They can be sterilized by, for example, filtration through a bacteria-retaining filter, by ionizing radiation (gamma photons for example), autoclaving, or by incorporating sterilizing agents in the form of sterile solid compositions that can be dissolved in sterile water, or some other sterile injectable medium immediately before use.

Liquid dosage forms useful for topical administration include pharmaceutically acceptable emulsions, lyophiles for reconstitution, microemulsions, solutions, suspensions, gels, syrups and elixirs. In addition to the active ingredient, the liquid dosage forms can contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (such as cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the topical compositions can also include adjuvants such as wetting agents, emulsifying and suspending agents, and preservative agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound can be mixed under sterile conditions with a pharmaceutically acceptable carrier, and with any preservatives or buffers that can be required.

The ointments, pastes, creams and gels can contain, in addition to an active compound, one or more excipients or carriers, such as animal and vegetable fats, oils, waxes, paraffins, starch, tragacanth, cellulose derivatives, polyethylene glycols, silicones, bentonites, silicic acid, talc, polymers, salts, and zinc oxide, or mixtures thereof. In some embodiments, the BT composition is in the form of an aqueous solution. In some embodiments, the excipient comprises a salt selected from sodium chloride or potassium chloride. In some embodiments, the excipient comprises sodium chloride.

In certain embodiments, the BT composition is a suspension of one or more BT compounds in TWEEN® (e.g. TWEEN 80®, polysorbate 80) and/or in a buffer (e.g. sodium phosphate buffer). For example, in some embodiments, the BT composition is a suspension of one or more BT compounds in from about 0.1% TWEEN 80® to about 1.0% TWEEN 80®, including all ranges therebetween. For example, the BT composition can be a suspension of one or more BT compounds in about 0.1% TWEEN 80®, about 0.2% TWEEN 80®, about 0.3% TWEEN 80®, about 0.4% TWEEN 80®, about 0.5% TWEEN 80®, about 0.6% TWEEN 80®, about 0.7% TWEEN 80®, about 0.8% TWEEN 80®, about 0.9% TWEEN 80®, or about 1% TWEEN 80®. In some embodiments, the BT composition is a suspension of one or more BT compounds in about 0.5% TWEEN 80®.

In a specific embodiment, the present invention may be a pharmaceutical composition comprising bismuth-thiol (BT) composition that comprises BisEDT suspended therein, wherein the BT composition comprises a plurality of microparticles. In a specific embodiment, the D90 of said microparticles is less than or equal to 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges in between. In some embodiments, the D90 of said microparticles is less than or equal to 3 μm. In some embodiments, the D90 of said microparticles is less than or equal to 2.9 μm. In some embodiments, the D90 of said microparticles is less than or equal to 2.8 μm. In some embodiments, the D90 of said microparticles is less than or equal to 2.7 μm. In some embodiments, the D90 of said microparticles is less than or equal to 2.6 μm. In some embodiments, the D90 of said microparticles is less than or equal to 2.5 μm. In some embodiments, the D90 of said microparticles is less than or equal to 2.4 μm. In some embodiments, the D90 of said microparticles is less than or equal to 2.3 μm. In some embodiments, the D90 of said microparticles is less than or equal to 2.2 μm. In some embodiments, the D90 of said microparticles is less than or equal to 2.1 μm. In some embodiments, the D90 of said microparticles is less than or equal to 2.0 μm. In one embodiment, the D90 of said microparticles is less than or equal to 1.9 μm. In one embodiment, the D90 of said microparticles is less than or equal to 1.6 μm. In one embodiment, the D50 of said microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In one embodiment, the D10 of said microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between.

In some embodiments, the pharmaceutical composition comprising bismuth-thiol (BT) composition comprises BisEDT suspended therein, wherein the BT composition comprises a plurality of microparticles, wherein the D90 of said microparticles is less than or equal to about 3 sm. In some embodiments, the pharmaceutical composition comprising bismuth-thiol (BT) composition comprises BisEDT suspended therein, wherein the BT composition comprises a plurality of microparticles, wherein the D90 of said microparticles is less than or equal to about 2 sm. In a specific embodiment, the pharmaceutical composition comprising bismuth-thiol (BT) composition comprises BisEDT suspended therein, wherein the BT composition comprises a plurality of microparticles, wherein the D90 of said microparticles is less than or equal to about 1.6 μm. In a specific embodiment, the BT composition comprises BisEDT at a concentration greater than about 0.1 mg/mL, about 0.05% to about 1.0% TWEEN 80®, about 0.05 to 40 mM sodium chloride, and optionally about 2 to 20 mM sodium phosphate at about pH 7.4. In another specific embodiment, the compositions described above can be administered to a subject for treating, preventing and/or lessening the severity of osteomyelitis in a subject.

In some embodiments, the composition is a suspension of microparticles having a volumetric mean diameter (VMD) from about 0.4 μm to about 5 μm. In some embodiments, at least 60%, 65%, 70%, 75%, 80%, 90%, or 95% of the microparticles have a VMD of from about 0.4 μm to about 5 μm, or from about 0.6 μm to about 2.5 μm, or from about 0.7 μm to about 4 μm, or from about 0.7 μm to about 3.5 μm, or from about 0.7 μm to about 3.0 μm, or from about 0.9 μm to about 3.5 μm, or from about 0.9 μm to about 3 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm and all ranges therebetween. In some embodiments, at least 60%, 65%, 70%, 75%, 80%, 90%, or 95% of the microparticles have a VMD of from about 0.6 μm to about 2.5 μm, or from about 0.8 μm to about 1.6 μm, or from about 0.9 μm to about 3.5 μm, or from about 0.9 μm to about 3 μm, or from about 0.9 μm to about 1.4 μm, or from about 1.0 μm to about 2.0 μm, or from about 1.0 μm to about 1.8 μm and all ranges therebetween. In some embodiments, the microparticles have a D90 of less than about 10 μm. For example, in some embodiments, the microparticles have a D90 of less than about 10 μm, 9 μm, 8 μm, 7 μm, 6 μm, 5 μm, 4 μm, 3 μm, 2 μm, or about 1 μm. In some embodiments, the microparticles have a D90 of less than about 3 μm. In some embodiments, the microparticles have a D90 ranging from about 1 μm to about 5 μm, or about 2 μm to about 6 μm, or about 2 μm to about 4 μm, or about 2 μm to about 3 μm, or about 1 μm to about 4 μm, or about 1 μm to about 3 μm.

In some embodiments, the pharmaceutical composition comprising bismuth-thiol (BT) composition comprises BisEDT suspended therein, wherein the BT composition comprises a plurality of microparticles, wherein the D90 of said microparticles is less than or equal to about 3 μm and the VMD is from about 0.6 μm to about 2.5 μm. In some embodiments, the pharmaceutical composition comprising bismuth-thiol (BT) composition comprises BisEDT suspended therein, wherein the BT composition comprises a plurality of microparticles, wherein the D90 of said microparticles is less than or equal to about 1.6 μm and the VMD is from about 1 μm to about 2 μM.

A variety of buffers may be used in the context of the present disclosure and will be readily apparent to a person having ordinary skill in the art. For example, in some embodiments, suitable buffers include sodium or potassium citrate, citric acid, phosphate buffers such as sodium phosphate, boric acid, sodium bicarbonate and various mixed phosphate buffers including combinations of Na₂HPO₄, NaH₂PO₄ and KH₂PO₄. In some embodiments, sodium phosphate buffer is used. In some embodiments, sodium citrate buffer is used. Without being bound by any particular theory, changes in airway surface liquid pH may contribute to the host defense defect in cystic fibrosis soon after birth. Changes in lung pH may impact the airway surface liquid environment, improve airway defenses, and alter the disease course. Accordingly, the formulation pH may vary from about 5 to about 10. In some embodiments, the formulation pH is about 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or about 10. In some embodiments, the formulation pH is about 7.4.

In some embodiments, the BT composition is a suspension of one or more BT compounds in about 0.5% TWEEN 80® in sodium phosphate buffer at a pH of about 7.4. In some embodiments, the one or more BT compounds are present in the composition at a concentration ranging from about 100 μg/mL to about 1000 mg/mL including all integers and ranges therebetween. For example, in some embodiments, the one or more BT compounds are present in the composition at a concentration ranging from about 100 μg/mL, 200 μg/mL, 300 μg/mL, 400 μg/mL, 500 μg/mL, 600 μg/mL, 700 μg/mL, 800 μg/mL, 900 μg/mL, 1000 μg/mL, 10 mg/mL, 25 mg/mL, 50 mg/mL, 100 mg/mL, 125 mg/mL, 150 mg/mL, 175 mg/mL, 200 mg/mL, 225 mg/mL, 250 mg/mL, 275 mg/mL, 300 mg/mL, 325 mg/mL, 350 mg/mL, 375 mg/mL, 400 mg/mL, 425 mg/mL, 450 mg/mL, 475 mg/mL, 500 mg/mL, 525 mg/mL, 550 mg/mL, 575 mg/mL, 600 mg/mL, 625 mg/mL, 650 mg/mL, 675 mg/mL, 700 mg/mL, 725 mg/mL, 750 mg/mL, 775 mg/mL, 800 mg/mL, 825 mg/mL, 850 mg/mL, 875 mg/mL, 900 mg/mL, 925 mg/mL, 950 mg/mL, 975 mg/mL, to about 1000 mg/mL. In some embodiments, the one or more BT compounds are present in the composition at a concentration ranging from about 100 μg/mL to about 1000 μg/mL.

In some embodiments, the composition osmolality may need to be further adjusted with an additive such as NaCl or TDAPS to achieve a desired osmolality. For example, in some embodiments, the osmolality of the composition is adjusted with sodium chloride to an osmolality ranging from about 100 mOsmol/kg to about 500 mOsmol/kg, including all integers and ranges therebetween. In some embodiments, the osmolality of the composition is from about 290 mOsmol/kg to about 310 mOsmol/kg. For example, in some embodiments, the osmolality of the composition is about 290 mOsmol/kg, 291 mOsmol/kg, 292 mOsmol/kg, 293 mOsmol/kg, 294 mOsmol/kg, 295 mOsmol/kg, 296 mOsmol/kg, 297 mOsmol/kg, 298 mOsmol/kg, 299 mOsmol/kg, 300 mOsmol/kg, 301 mOsmol/kg, 302 mOsmol/kg, 303 mOsmol/kg, 304 mOsmol/kg, 305 mOsmol/kg, 306 mOsmol/kg, 307 mOsmol/kg, 308 mOsmol/kg, 309 mOsmol/kg, to about 310 mOsmol/kg. In some embodiments, the osmolality is about 300 mOsmol/kg.

In some embodiments, the BT composition is a suspension of BisEDT in TWEEN® (e.g. TWEEN 80®) in a buffer (e.g. sodium phosphate buffer). In some embodiments, the BT composition is a suspension of BisEDT in about 0.5% TWEEN 80® in a sodium phosphate buffer at a pH of about 7.4. In some embodiments, the BT composition is a suspension of BisEDT in about 0.5% TWEEN 80® in a sodium phosphate buffer at a pH of about 7.4, wherein the composition has an osmolality of about 300 mOsmol/kg (e.g. adjusted to 300 mOsmol/kg with sodium chloride). In some embodiments, the BisEDT is present at a concentration of about 100 μg/mL, 250 μg/mL, 500 μg/mL, 750 μg/mL, 1000 μg/mL, 2.5 mg/mL, 10 mg/mL, 25 mg/mL, 50 mg/mL, 75 mg/mL, or about 100 mg/mL.

### Methods of Use

The current standard of care to treat orthopaedic infection is challenged by widespread antibiotic resistance and the presence of recalcitrant biofilms. For example, when an orthopaedic procedure involves implantation of a device, the presence of biofilm on the device and surrounding tissue contribute to the persistence and virulence of infection. Consequently, outcomes associated with such infections are often poor and can lead to (a) chronic/recurrent infection; (b) repeated hospitalizations; (c) repeated surgeries; (d) multiple course of systemic antibiotics; (e) loss of function/disability/amputation; and (f) death. Local therapies that eliminate established biofilm or prevent its formation, coupled with eradicating causative pathogens, potentially promote more rapid/complete resolution of orthopaedic infections.

Bismuth-thiol (BT) compounds, such as BisEDT, are a new class of anti-infective agent that has demonstrated broad-spectrum in vitro and in vivo antimicrobial activity against a variety of difficult-to-treat antibiotic-resistant bacteria including MRSA, methicillin-resistant Staphylococcus epidermidis, antibiotic-resistant Pseudomonas aeruginosa, extended-spectrum beta lactamase-positive Klebsiella pneumoniae, and antibiotic-resistant Enterobacter, to name a few. These compounds are also highly active against antibiotic-resistant infections caused by biofilms and exhibit low resistance potential. As such, BT compounds and compositions thereof have been established as effective treatments for wound, cystic fibrosis, diabetic foot infections and other complicated infections as described in International Publication Nos. WO2010/091124, WO2011/097347, WO2020/028558, and WO2020/028561; U.S. Pat. Nos. 8,389,021, 9,028,878, 10,835,510, 10,960,012; and U.S. Publication Nos. 2021/0260017, 2020/0038361, and 2020/0046650, each of which is incorporated herein by reference in its entirety for all purposes.

To further expand the therapeutic utility of bismuth-thiol compounds, the present disclosure describes the use of BT compounds in treating, preventing, and reducing the effects of osteomyelitis and other conditions.

Accordingly, in some embodiments, the present disclosure provides methods for treating or preventing osteomyelitis, a bone infection, or infection near a bone or orthopaedic device inserted in a subject, comprising administering to a subject in need thereof a therapeutically effective amount of a composition comprising a bismuth-thiol compound disclosed herein, wherein the composition is applied directly to the site of an infection (e.g., a bone infection).

Topical treatment provides the advantages of avoiding systemic adverse effects, providing increased target site concentration, and allowing the use of agents not available for systemic therapy. In some embodiments, mechanical debridement may be used to improve topical treatment because it reduces the bioburden of bacteria present and also opens a time-dependent therapeutic window for topical antimicrobial therapy (TAT) (Wolcott R D, et al. 2010. J Wound Care 19:320-328). Nevertheless, to date, no TAT agent has been proven to be effective for treating DFI (Nelson E A, et al. 2006. Diabet Med 23:348-359).

In some embodiments, the osteomyelitis results from a puncture wound infection, surgical infection or an infection of an open fracture. In some embodiments, the osteomyelitis results from a secondary infection from seeding of bacterial. In some embodiments, the osteomyelitis is acute, chronic, or chronic or acute-one-chronic osteomyelitis. In some embodiments, the osteomyelitis results from a bloodstream infection (bacteremia), wherein bacteria are deposited in a focal area of the bone. In some embodiments, the osteomyelitis results from a chronic open wound or soft tissue infection that extends down to the bone surface. In some embodiments, the osteomyelitis results from a diabetic foot infection. In some embodiments, a bone of a subject is infected with one or more of the following bacterial pathogens: Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asacchamlydcus, Veillonella atypia, Anaerococcus vaginalis. In some embodiments, the bone infection is caused by one or more of the following pathogens: Corynebacterium jeikeium, Corynebacterium, non-speciated, Actinomyces turicensis, Corynebacterium amycolatum, Corynebacterium resistens, Corynebacterium simulans, Dermabacter hominis, Staphylococcus epidermidis, Staphylococcus aureus, MRSA, Staphylococcus aureus, MSSA, Staphylococcus lugdunensis, Enterococcus faecalis, Granulicatella, non-speciated, Staphylococcus arlettae, Staphylococcus haemolyticus, Staphylococcus hominis, Staphylococcus pasteuri, Staphylococcus warneri, Streptococcus oralis, Enterobacter cloacae, Serratia marcescens, Escherichia coli, Klebsiella oxytoca, Pseudomonas aeruginosa, Stenotrophomonas maltophilia, Cutibacterium (Propionibacterium) acnes, Finegoldia magna, Anaerococcus murdochii, Anaerococcus, non-speciated, Clostridium sphenoides, Peptoniphilus gorbachii, Prevotella bergensis, and/or Candida parapsilosis.

Biofilms of S. aureus and other bacteria that are present in the bone infections of patients increase the difficulty of successful infection management and reduction. Combinations of such bacteria forming multispecies biofilms containing e.g. S. aureus have demonstrated greater resistance, virulence and pathogenicity than comparable single-species biofilms. The presence of such complex biofilms in bone infections of patients is considered to be at least partly responsible for the recalcitrant nature of these infections.

In some embodiments, the bacterial pathogen exhibits resistance to one or more antibiotics. Of particular concern in osteomyelitis are the methicillin-resistant Staphylococcus aureus strains (MRSA). MRSA remained an uncommon occurrence in hospital setting until the 1990's, when there was an explosion in MRSA prevalence in hospitals. MRSA now is considered endemic to hospitals, especially in the UK (Johnson A P et al. 2001 J. Antimicrobial Chemotherapy 48(1): 143-144). Moreover, MRSA presents a new threat in diabetic foot infections (Retrieved Jan. 17, 2009, from CDC: Centers for Disease Control and Prevention Web site). The ulcers and open sores that can occur in diabetic feet put patients at risk for contracting MRSA, and recent studies show evidence of MRSA impairing healing when present in the diabetic wound (Bowling F L, et al. 2009 Curr Diab Rep 9(6):440-444). See also, Kosinski, M A, et al. 2010. Expert Rev AntiInfect Ther. 8(11):1293-1305. In some embodiments, a bacterial pathogen is resistant to known standards of antibiotic care, including, but not limited to, amikacin, methicillin, vancomycin, nafcillin, gentamicin, metronidazole, Piperacillin/Tazobactam, ampicillin, chloramphenicol, doxycycline, tobramycin, levofloxacin, cephalosporins (e.g. cephalexin, cefoxitin, ceftizoxime, ceftibiprole, ceftazidime, ceftaroline), penicillin/β-lactamase inhibitor combinations (e.g. amoxicillin/clavulanate, ampicillin/sulbactam, piperacillin/tazobactam, and ticarcillin/clavulanate), carbapenems (e.g. imipenem/cilastatin, ertapenem), fluoroquinolones (e.g. ciprofloxacin, moxifloxacin), clindamycin, linezolid, daptomycin, tigecycline, and vancomycin.

Long-term, repeated treatment with antibiotics to treat bone infections may result in development of antibiotic-resistance, characterized by the presence of microbial biofilms. Recent research has repeatedly demonstrated a correlation between multi-drug resistant (MDR) bacteria, and stronger, more prolific biofilm-forming capabilities. Bacteria within biofilms are protected from antibiotics, which increases the minimal inhibitory concentration of such antibiotics.

The BT compositions of the present disclosure have activity against a plurality of bacterial and fungal strains. In some embodiments, the BT compositions have activity against a plurality of strains including but not limited to Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. Accordingly, some embodiments of the present disclosure provide methods of treating and/or preventing infections associated with Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis in both humans and animals using the BT compositions. In other aspects, the present disclosure provides methods of treating and/or preventing infections associated with related species or strains of these bacteria. In some embodiments, the bacterial infection is an infection associated with diabetic lower extremity infections, such as diabetic foot infections.

Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis are responsible for many severe opportunistic infections, particularly in individuals with compromised immune systems, including diabetic patients. The pharmaceutical compositions of the present disclosure are contemplated for treating and/or preventing any infection associated with Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis or associated with other species or strains of bacteria, including, but not limited to, infections of the skin, infections in and around wounds, chronic ulcers, ulcers associated with burn wounds, postoperative infections, infections associated with catheters and surgical drains, and infections of the blood. In some embodiments, the pharmaceutical compositions of the present disclosure find use in treating and/or preventing bacterial infections associated with areas of non-intact skin, including but not limited to, infections associated with cutaneous ulcers, such as diabetic foot ulcers, skin lesions, vesicles, cysts, blisters, bullae, open sores such as decubitus ulcers (bed sores) and other pressure sores, chronic ulcers, cellulitis and sores associated therewith, erysipelas and lesions associated therewith, wounds, burns and wounds associated therewith, carbuncles, or other conditions where the skin is damaged, cracked, broken, breached, and/or otherwise compromised.

In embodiments described herein, the BT compositions may be used to treat an infection (e.g. osteomyelitis, bone infection, or infection near a bone or orthopaedic device inserted in a subject) of one or more of the following bacterial pathogens:
Acinetobacter baumanii
Acinetobacter junii
Anaerococcus lactolyticus
Anaerococcus vaginalis
Anaerococcus murdoch
Anaerococcus tetradius
Anaerococcus hydrogenalis
Actinobaculum massiliense
Actinobaculum schaalii
Actinomyces europaeus
Actinomyces hominis
Actinomyces neuii
Actinomyces radingae
Alcaligenes faecalis
Abiotrophia paraadiacens
Bacteroides fragilis
Bulleidia extructa
Bilophila wadsworthia
Campylobacter ureolyticus
Citrobacter murliniae
Clostridium saccharogumia
Clostridium novyi
Corynebacterium accolens
Corynebacterium amycolatum
Corynebacterium aurimucosum
Corynebacterium freiburgense
Corynebacterium hansenii
Corynebacterium jeikeium
Corynebacterium mycetoide
Corynebacterium simulans
Corynebacterium Tuberculostearicum
Corynebacterium xerosis
Corynebacterium striatum
Dermabacter hominis
Dialister invisus
Dialister propionicifaciens
Dialister micraerophilus
Dialister pneumosintes
Delftia acidovorans
Enterobacter aerogenes
Enterobacter cloacae
Enterobacter hormaechei
Enterococcus faecalis
Enterococcus canintestini
Echerichia coli
Escherichia fergusonii
Escherichia vulneris
Enterococcus avium
Enterococcus gallinarum
Enterococcus casseliflavus
Eggerthella lenta
Finegoldia magna
Fusobacterium canifelinum
Fusobacterium nucleatum
Fusobacterium periodontium
Granulicatella adiacens
Gemella morbillorum
Globicatella sanguinis
Haemophilus parainfluenzae
Haemophilus segnis
Helcococcus kunzii
Helcococcus kunzii
Klebsiella oxytoca
Kocuria atrinae
Leclercia adecarboxylata
Mobiluncus curtisii
Moryella indoligenes
Morganella morganii
Negativicoccus succinicivorans
Peptoniphilus harei
Peptoniphilus gorbachii
Peptoniphilus ivorii
Peptoniphilus lacrimalis
Peptoniphilus olseni
Peptoniphilus asacchrolyticus
Parvimonas micra
Peptococcus niger
Peptostreptococcus anaerobius
Peptostreptococcus stomatis
Porphyromonas asaccharolyica
Porphyromonas bennonis
Porphyromonas somerae
Porphyromonas uenonis
Porphyromonas levii
Prevotella timonensis
Prevotella bergensis
Prevotella buccalis
Prevotella corporis
Prevotella disiens
Prevotella intermedia
Prevotella nanceiensis
Pseudomonas indica
Pseudomonas otitidis
Psychrobacter lutiphocae
Proteus myxofaciens
Proteus hauseri
Providencia rettgeri
Providencia stuartii
Staphylococcus aureus
Staphylococcus epidermidis
Staphylococcus carnosus
Staphylococcus chromogenes
Staphylococcus devriesei
Staphylococcus hominis
Staphylococcus lugdunensis
Serratia nematodiphila
Stenotrophomonas maltophilia
Staphylococcus pettenkoferi
Staphylococcus capitis
Staphylococcus saprophyticus
Streptococcus agalactiae
Streptococcus anginosus
Streptococcus canis
Streptococcus dysgalactiae
Streptococcus infantarius
Streptococcus oralis
Serratia grimesii
Stenotrophomonas pavanii
Salmonella enterica
Trueperella bernardiae
Varibaculum cambriense
Veillonella atypica
Veillonella parvula
Veillonella dispar
Veillonella rogosae
Acinetobacter calcoaceticus
Acinetobacter lwoffii
Anaerococcus prevotii
Bacteroides caccae
Bacteroides distasonis
Bacteroides ovatus
Bacteroides stercoris
Bacteroides thetaiotaomicron
Bacteroides uniformis
Bacteroides vulgatus
Corynebacterium striatum
Clostridium innocuum
Clostridium perfringens
Clostridium ramosum
Pluralibacter gergoviae
Fusobacterium mortiferum
Finegoldia magna
Klebsiella oxytoca
Klebsiella pneumoniae
Pseudomonas aeruginosa
Peptococcus magnus
Prevotella bivia
Prevotella melaninogenica
Porphyromonas asaccharolytica
Peptostreptococcus asaccharolyticus
Peptostreptococcus micros
Parvimonas micra
Proteus mirabilis
Staphylococcus haemolyticus
Staphylococcus simulans
Staphylococcus saprophyticus
Streptococcus pneumoniae
Streptococcus agalactiae
Streptococcus mitis
Streptococcus milleri
Streptococcus dysgalactiae
Streptococcus canis
Serratia marcescens
Serratia liquefaciens
Stenotrophomonas maltophila
Epidermolysis bullosa

In embodiments described herein, the BT compositions may be used to treat an infection (e.g. osteomyelitis, bone infection, or infection near a bone or orthopaedic device inserted in a subject,) of one or more of the following fungal pathogens: Candida spp., Cladosporium spp., Aspergillus spp., Penicillium spp., Alternaria spp., Pleospora spp., Fusarium spp, Candida lusitaniae, Candida parapsilisis, and Candida albicans.

In some embodiments, the BT compositions of the present disclosure find use in treating bone infections that result from the development of chronic ulcers. Chronic ulcers may arise from wounds caused by a variety of factors, especially in patients with impaired blood circulation, for example, caused by cardiovascular issues or external pressure from a bed or a wheelchair. More than 8 million patients are diagnosed with chronic skin ulcers each year in the United States alone (Harsha, A. et al., 2008, Journal of Molecular Medicine, 86(8): 961-969), which costs more than 10 billion dollars per year (Margolis, D J, et al., 2002, Journal of the American Academy of Dermatology 46(3): 381-386). Chronic ulcers may develop in the mouth, throat, stomach, and skin. Chronic skin ulcers include diabetic ulcers, venous ulcers, radiation ulcers, and pressure ulcers, the three major categories of chronic skin ulcers being diabetic ulcers, venous stasis ulcers, and pressure ulcers. Chronic ulcers can cause the loss of the integrity of large portions of the skin, even leading to morbidity and mortality.

In some embodiments, the BT compositions of the present disclosure find use in treating bone infections that result from the development of diabetic lower extremity infections, such as diabetic foot infections. Diabetic foot infection is one of the major complications of diabetes mellitus, occurring in about 15% of all diabetic patients and resulting in about 85% of all lower leg amputations. (Brem, et al., J. Clinical Invest, 2007, 117(5):1219-1222). Diabetes mellitus impedes the normal steps of the wound healing process, such that diabetic foot infections can become associated with non-healing, chronic cutaneous ulcers.

In some embodiments, the bone infections disclosed herein result from the persistence of a chronic wound. A chronic wound represents a failure of the normal processes of acute wound healing. Wound healing has traditionally been divided into three distinct phases: inflammation, proliferation and remodeling. The inflammatory phase of wound healing begins at the time of injury by forming a clot via a platelet plug, thereby initiating a response from neutrophils and macrophages. Neutrophils initially clear the wound of bacteria and debris by releasing a variety of proteases and reactive oxygen free radicals. Macrophages are then attracted to the wound site by chemoattractants and subsequently release their own chemoattractants to stimulate fibroblasts and more macrophages. During the proliferation phase, fibroblasts initiate epithelialization, angiogenesis, and collagenation. Epithelialization generally occurs from the basement membrane if it remains intact and from the wound margins if not intact. Fibroblasts synthesize type III collagen during this phase and transform into myofibroblasts, which help to stimulate wound contraction. During the remodeling phase, type III collagen begins to be replaced by type I collagen. Collagen is woven into an organized, cross-linked network whose strength approaches 80% of the original uninjured tissue.

There are many factors that can stall the three-phase healing process and convert an acute wound into a chronic wound and then into a bone infection. These may include a low proliferative capacity of the fibroblasts, downregulation of receptors, reduced growth factors, or the absence of a suitable protein matrix in the dermis. Further, poor perfusion and/or nutrition can cause a wound to halt in the inflammatory phase and lead to excessive build-up of exudate in the wound. A chronic ulcer can be considered to be a non-healing area of non-intact skin, such as an area of non-intact skin that fails to follow the normal processes of wound healing, e.g., as described above, and/or that fails to respond, or fails to respond appropriately, to initial treatment. A chronic ulcer on the skin may be characterized as a wound lesion lasting more than four weeks, without remarkable healing tendency or as a frequently recurrent wound (Fonder, M. et al., 2012, Journal of the American Academy of Dermatology 58(2): 185-206). A chronic wound may appear with red granulation and yellow pus, a dim purple skin around granular tissues, or gray-white and swelling granulation. Standard care procedures for chronic skin ulcer include, e.g., the following: removal of necrotic or infected tissue; establishment of adequate blood circulation; maintenance of a moist wound environment; management of wound infection; wound cleansing; and nutritional support, including blood glucose control for subjects with diabetic ulcers. For example, in the diabetic patient, poor control of blood glucose levels allows bacteria to grow more rapidly in a wound; further still, neural degeneration in diabetes means the condition may not be painful and thus go undetected, at least initially. Chronic ulcers, including diabetic foot ulcers, often become further infected with opportunistic bacteria, leading to exacerbation of the condition. Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis are associated with such infections.

In some embodiments, the pharmaceutical composition of the present disclosure is formulated for use in methods of treating and/or preventing bacterial infections caused by Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the pharmaceutical composition of the present disclosure is formulated for use in methods of treating and/or preventing bacterial infections caused by Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some other embodiments, the pharmaceutical composition of the present disclosure is formulated for use in methods of treating and/or preventing bacterial infections caused by bacteria other than Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asacchamlydcus, Veillonella atypia, Anaerococcus vaginalis.

In some embodiments, the pharmaceutical composition of the present disclosure is formulated for use in methods of treating and/or preventing infections caused by the infection is caused by one or more of the following pathogens Corynebacterium jeikeium, Corynebacterium, non-speciated, Actinomyces turicensis, Corynebacterium amycolatum, Corynebacterium resistens, Corynebacterium simulans, Dermabacter hominis, Staphylococcus epidermidis, Staphylococcus aureus, MRSA, Staphylococcus aureus, MSSA, Staphylococcus lugdunensis, Enterococcus faecalis, Granulicatella, non-speciated, Staphylococcus arlettae, Staphylococcus haemolyticus, Staphylococcus hominis, Staphylococcus pasteuri, Staphylococcus warneri, Streptococcus oralis, Enterobacter cloacae, Serratia marcescens, Escherichia coli, Klebsiella oxytoca, Pseudomonas aeruginosa, Stenotrophomonas maltophilia, Cutibacterium (Propionibacterium) acnes, Finegoldia magna, Anaerococcus murdochii, Anaerococcus, non-speciated, Clostridium sphenoides, Peptoniphilus gorbachii, Prevotella bergensis, and/or Candida parapsilosis.

In some embodiments, the present disclosure provides methods of treating and/or preventing osteomyelitis, comprising administering to a subject in need thereof a therapeutically or prophylactically effective amount of a pharmaceutical composition of the present disclosure. In some embodiments, administration comprises topical administration to the area of non-intact skin to prevent the development of osteomyelitis. In some embodiments, topical administration follows debridement of the area to be treated.

In some embodiments, treatment of osteomyelitis includes debridement to avoid relocation of an infection to the bone. Debridement can be accomplished by a number of approaches. Surgical debridement involves cutting away dead tissues of the wound or other area of non-intact skin. Mechanical debridement uses various methods to loosen and remove wound debris, such as a pressurized irrigation device, a whirlpool water bath, ultrasound, larval maggots, or specialized dressings. Autolytic debridement enhances the body's natural process of recruiting enzymes to break down dead tissue, for example, using an appropriate dressing that keeps the wound moist and clean. Enzymatic debridement uses chemical enzymes and appropriate dressings to further aid in the break down dead tissues at the site of a wound or other area of non-intact skin.

Debridement improves topical treatment because it reduces the bioburden of bacteria present and also opens a time-dependent therapeutic window for topical antimicrobial therapy (TAT) (Wolcott R D, et al. 2010. J Wound Care 19:320-328). Regarding the timing for debridement, early or immediate debridement is preferred to delayed debridement once this treatment option is chosen in the management of a wound. Further, multiple debridements during wound management may be indicated (Wolcott R D, et al. 2009. J Wound Care 18(2):54-6). For example, in some embodiments, debridement precedes topical application of a BT composition of the present disclosure, and is repeated before every administration of the BT composition. In some embodiments, debridement is performed only before every other administration of the BT composition, or only before every 3^{rd}, 4^{th}, 5^{th}, or 6^{th} administration of the BT composition. In some embodiments, debridements are performed less frequently than the application of the BT composition, for example, once a week. Accordingly, if the BT composition is applied daily, the patient will not get debridement every time it is applied. In some embodiments, whether or not wound debridement is performed before topical administration of a BT composition of the present disclosure is within the clinical judgment of a health care practitioner treating the wound, e.g., the physician, physician's assistant, or emergency medical personnel.

The BT compositions of the present disclosure can find use in the treatment, management, control, and/or prevention of osteomyelitis that results from chronic ulcers, including diabetic foot infections and cutaneous ulcers associated therewith. In other embodiments, BT compositions of the present disclosure find use in the treatment, management, control, and/or prevention of osteomyelitis (e.g. bacterial and/or fungal) that results from infected areas of non-intact skin, such as a cellulitis sore, an erysipelas lesion, a decubitus ulcer, a burn wound, a traumatic wound, and a pressure sore. In some such embodiments, the composition used may be a topical composition, formulated for topical administration, e.g., for direct application to an area of non-intact skin, such as described above.

BT compositions of the present disclosure will comprise a therapeutically and/or prophylactically effective amount of one of more BT compounds (e.g. BisEDT), as described herein. A therapeutically and/or prophylactically effective amount refers to an amount required to bring about a therapeutic and/or prophylactic benefit, respectively, in a subject receiving said amount A therapeutically and/or prophylactically effective amount will depend on the particular formulation, route of administration, condition being treated, whether other agents or therapies are used in combination with methods of the present disclosure, and other factors.

In some embodiments of the methods for treating osteomyelitis, bone infection, or infection near a bone or orthopaedic device inserted in a subject, the subject experiences one or more of the following outcomes following the completion of dosing: less reinfection/relapse for the 12 weeks after start of treatment; resolution or improvement in signs and/or symptoms of infection that include redness, swelling, induration, exudate, pain, warmth (at site of infection) or fever; improved quality of life; eradication of insulting pathogens and/or biofilm; reduced need for concurrent systemic antibiotics.

In some embodiments, administration of a therapeutically effective amount of a BT composition, in accordance with the present disclosure, results in improved wound closure (partial or full), such as a reduction in the area of non-intact skin (wound area) compared to the area before initiation of treatment, which serves to prevent the development of osteomyelitis. Wound area can be expressed as a percentage of the initial wound area, at one or more time points after initiation of treatment. For example, in some embodiments, wound area decreases by at least about 20%, at least about 30%, at least about 40%, at least about 50%, at least about 60%, at least about 70%, at least about 80%; or at least about 90% over a course of treatment with a BT composition of the present disclosure. In some specific embodiments, the decrease in wound area occurs at least by day 1 after treatment initiation (t1), day 2 after treatment initiation (t2), day 3 after treatment initiation (t3), day 4 after treatment initiation (t4), day 5 after treatment initiation (t5), day 6 after treatment initiation (t6), day 7 after treatment initiation (t7), day 8 after treatment initiation (t8), day 9 after treatment initiation (t9), day 10 after treatment initiation (t10), day 12 after treatment initiation (t12), day 15 after treatment initiation (t15), day 20 after treatment initiation (t20), day 25 after treatment initiation (t25), or day 30 after treatment initiation (t30).

In some embodiments of the methods for treating osteomyelitis, the BT composition comprises one or more BT compounds selected from BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-Pyr/Tol, Bis-Pyr/Ery, bismuth-1-mercapto-2-propanol, and Bis-EDT/2-hydroxy-1-propanethiol and the composition is a suspension of microparticles comprising said BT compounds having a volumetric mean diameter (VMD) from about 0.4 μm to about 5 μm. In some embodiments, the BT compound is BisEDT.

In some embodiments of the methods for treating osteomyelitis, the BT composition comprises BisEDT and the applied BisEDT is present on the surface at a concentration greater than about 20 μg/cm². In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH 7.4.

In some embodiments of the methods for treating osteomyelitis, after administration of the BT composition, one or more of the following occurs: (i) reducing and or dispersing a microbial (e.g. bacterial and/or fungal) biofilm, (ii) impairing growth or formation of a microbial (e.g. bacterial and/or fungal) biofilm, and (iii) preventing reformation or spread of a microbial (e.g. bacterial and/or fungal) biofilm. In some embodiments, the BT composition treats, manages, and/or lessens the severity of the diabetic foot infection by one or both of: (i) prevention of the infection by the bacterial or fungal pathogen; and/or (ii) reduction of the bacterial or fungal pathogen. In some embodiments, the BT composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm formation by the bacterial or fungal pathogen; (iv) inhibition of biofilm or microbial pathogen invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement.

BT compounds are known broad-spectrum antimicrobial (and anti-biofilm) small molecule drug products for the treatment of chronic, ultimately life-threatening infections. Its efficacy extends to Gram-positive (aerobic and anerobic), antibiotic-resistant pathogens including methicillin-resistant Staphylococcus aureus (MRSA, including community-associated [CA]-MRSA), methicillin-resistant Staphylococcus epidermidis (MRSE), and vancomycin-resistant Enterococcus (VRE). BT compounds are also potent against Multi-drug-resistant (MDR) Gram-negative pathogens (aerobic and anerobic) including Pseudomonas aeruginosa, Escherichia coli, Klebsiella pneumoniae (including, in all of the afore-mentioned bacteria, carbapenem-resistant strains), and Acinetobacter baumannii.

BT compounds have the dual ability to overcome a) a very diversified spectrum of antibiotic resistance profiles (due to evolution/diversification driven by persistence, time and isolation in many different anatomical regions, and b) antibiotic-resistant and MDR biofilms.

In some embodiments, the infection contains one or more bacterial or fungal pathogens. In some embodiments, the disclosed methods comprise treating the DFI-related infection. In some embodiments, the disclosed methods comprise managing the DFI-related infection. In some embodiments, the disclosed methods comprise lessening the severity of the DFI-related infection.

In some embodiments, the bismuth-thiol composition comprises a plurality of microparticles that comprise a bismuth-thiol (BT) compound, substantially all of said microparticles having a volumetric mean diameter of from about 0.4 μm to about 5 μm, and wherein the BT compound comprises bismuth or a bismuth salt and a thiol-containing compound. In some embodiments, the bismuth salt is bismuth nitrate, bismuth subnitrate, or bismuth chloride. In some embodiments, the thiol-containing compound comprises one or more agents selected from 1,2-ethane dithiol, 2,3-dimercaptopropanol, pyrithione, dithioerythritol, 3,4 dimercaptotoluene, 2,3-butanedithiol, 1,3-propanedithiol, 2-hydroxypropanethiol, 1-mercapto-2-propanol, dithioerythritol, dithiothreitol and alpha-lipoic acid. In some embodiments, at least 70% of the microparticles have a volumetric mean diameter of from about 0.4 μm to about 3 μm, or from 0.6 μm to about 2.9 μm, or from 0.6 μm to about 2.5 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments, the BT composition comprises one or more BT compounds selected from

bismuth-2,3-dimercaptopropanol (2:3 molar ratio, BisBAL)
bismuth-dithioerythritol (2:3 molar ratio, BisERY)
bismuth-4-methyl-1,2-benzenedithiol (2:3 molar ratio, BisTOL)
bismuth-2,3-butanedithiol (BisBDT)
bismuth-2,3-butanedithiol, 2-mercaptopyridine N-oxide (2:1:2 molar ratio, BisBDT/PYR)
bismuth-2,3-dimercaptopropanol, 2-mercaptopyridine N-oxide (2:1:2 molar ratio,
BisBAL/PYR)
bismuth-1,2-ethanedithiol, 2-mercaptopyridine N-oxide (2:1:2 molar ratio, BisEDT/PYR)
bismuth-4-methyl-1,2-benzenedithiol, 2-mercaptopyridine N-oxide (2:1:2 molar ratio,
BisTOL/PYR)
bismuth-1,3-propanedithiol, 2-mercaptopyridine N-oxide (2:1:2 molar ratio, BisPDT/PYR)
bismuth-dithioerythritol, 2-mercaptopyridine N-oxide (2:1:2 molar ratio, BisERY/PYR)
bismuth-1-mercapto-2-propanol, 1,2-ethanedithiol (1:1:1 molar ratio, BisHPT/EDT)
bismuth with ethanedithiol and 2-mercaptobenzoimidazole (BisEDT/2MBI (1:1))
bismuth with ethanedithiol and 2-mercaptopyrimidine (BisEDT/SPN (2MPMD) (1:1))
bismuth with ethanedithiol and 3-mercapto-1,2,4-triazole (BisEDT/3MTZ (1:1))
bismuth with ethanedithiol and 1-propane thiol (BisEDT/PT (1:1))
bismuth with ethanedithiol and cysteamine (BisEDT/CSTMN (1:1))
bismuth with ethanedithiol and 3-mercaptopropionic acid (BisEDT/3MPA (1:1))
bismuth with lipoic acid (reduced) (BisALA (BisLipo) (1:1.5))
bismuth with 2-mercaptolpyridine N-oxide and 2-mercaptobenzoimidazole (BisPYR/2MBI
(1:1))
bismuth with 2-mercaptolpyridine N-oxide and cysteamine (BisPYR/CSTMN (1:1))
bismuth with 2,3-dimercapto-1-propanol and 2-mercaptobenzoimidazole (BisBAL/2MBI
(1:1))
bismuth with 2,3-dimercapto-1-propanol and cysteamine (BisBAL/CSTMN (1:1))
bismuth with 3,4 dimercapto toluene and 2-mercaptobenzoimidazole (BisTOL/2MBI (1:1))
bismuth with 3,4 dimercapto toluene and cysteamine (BisTOL/CSTMN (1:1))
bismuth with 2-mercapto pyridine (BisEDT/MPYR)

In some embodiments, the bismuth thiol compound is BisEDT, which has the following structure:

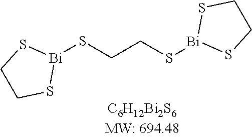

In some embodiments of the methods for treating osteomyelitis, the administered BT composition is present on the surface at a concentration from about 1 μg/cm² to about 1,000,000 μg/cm² (e.g. about 1 μg/cm² to about 10,000 μg/cm²), including all integers and ranges therebetween. In some embodiments, the administered BT composition is present on the surface at a concentration from about 50 μg/cm² to about 200 μg/cm². In some embodiments, the applied BT composition is present on the surface at a concentration from about 250 μg/cm² to about 5,000 μg/cm². For example, in some embodiments, the bismuth thiol compound in the BT composition is BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, the present disclosure provides methods of treating or preventing a bacterial infection in a subject in need thereof comprising administering to the subject a therapeutically effective amount of the pharmaceutical composition as described herein (e.g. a BisEDT-containing composition). In some embodiments, the bacterial infection is an infection by one or more of Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the pharmaceutical composition is administered topically. In some embodiments, the infection is an infection caused by one or more of Corynebacterium jeikeium, Corynebacterium, non-speciated, Actinomyces turicensis, Corynebacterium amycolatum, Corynebacterium resistens, Corynebacterium simulans, Dermabacter hominis, Staphylococcus epidermidis, Staphylococcus aureus, MRSA, Staphylococcus aureus, MSSA, Staphylococcus lugdunensis, Enterococcus faecalis, Granulicatella, non-speciated, Staphylococcus arlettae, Staphylococcus haemolyticus, Staphylococcus hominis, Staphylococcus pasteuri, Staphylococcus warneri, Streptococcus oralis, Enterobacter cloacae, Serratia marcescens, Escherichia coli, Klebsiella oxytoca, Pseudomonas aeruginosa, Stenotrophomonas maltophilia, Cutibacterium (Propionibacterium) acnes, Finegoldia magna, Anaerococcus murdochii, Anaerococcus, non-speciated, Clostridium sphenoides, Peptoniphilus gorbachii, Prevotella bergensis, and/or Candida parapsilosis.

In some embodiments of the methods for treating or preventing osteomyelitis, bone infection, or infection near a bone or orthopaedic device inserted in a subject, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for preventing osteomyelitis near a wound area, the wound area is from about 0.1 cm² to about 250 cm², including all integers and ranges therebetween. For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods for treating a bacterial infection, such as osteomyelitis, a bone infection, or infection near a bone or orthopaedic device inserted in a subject, comprising administering to a subject in need thereof a therapeutically effective amount of a composition comprising a bismuth-thiol compound, wherein the composition is applied directly to the infection (e.g. a surface of an infected bone). In some embodiments, the bacterial infection is osteomyelitis. In some embodiments, the bacterial infection comprises one or more of the following bacterial pathogens: Staphylococcus aureus, MRSA, Escherichia coli, Pseudomonas aeruginosa, Citrobacter spp., Klebsiella oxytoca, Proteus spp, Mobiluncus spp., Gardenella spp., Atopibium spp., S. epidermidis, Enterococcus faecalis, Coagulase-negative Staphylococcus spp., Streptococcus spp., Corynebacterium spp., Proteus mirabilis, Bacteroides spp., Peptostreptococcus spp., Propionibacterium spp., Clostridium spp., Peptococcus spp., Prevotella spp., Finegoldia spp., Propionibacterium acnes, S. dysgalactiae, Serratia spp., Rhodopseudomonas spp., Bacteroides fragilis, Morganella morganii, Hemophilus spp., Enterococcus spp., Stenotrophomonas spp., Pseudomonas spp., Stenotrophomonas maltophilia, Enterobacter cloacae, Sphingomonas sp., Acinetobacter spp., Anerococcus spp., Dialister spp., Peptoniphilus spp., Finegoldia magna, Peptoniphilus asaccharolyticus, Veillonella atypia, Anaerococcus vaginalis. In some embodiments, the infection comprises one or more of the following pathogens: Corynebacterium jeikeium, Corynebacterium, non-speciated, Actinomyces turicensis, Corynebacterium amycolatum, Corynebacterium resistens, Corynebacterium simulans, Dermabacter hominis, Staphylococcus epidermidis, Staphylococcus aureus, MRSA, Staphylococcus aureus, MSSA, Staphylococcus lugdunensis, Enterococcus faecalis, Granulicatella, non-speciated, Staphylococcus arlettae, Staphylococcus haemolyticus, Staphylococcus hominis, Staphylococcus pasteuri, Staphylococcus warneri, Streptococcus oralis, Enterobacter cloacae, Serratia marcescens, Escherichia coli, Klebsiella oxytoca, Pseudomonas aeruginosa, Stenotrophomonas maltophilia, Cutibacterium (Propionibacterium) acnes, Finegoldia magna, Anaerococcus murdochii, Anaerococcus, non-speciated, Clostridium sphenoides, Peptoniphilus gorbachii, Prevotella bergensis, and/or Candida parapsilosis.

In some embodiments of the methods for treating a microbial (e.g. bacterial and/or fungal) infection, the BT composition comprises one or more BT compounds selected from BisBAL, BisEDT, Bis-dimercaprol, BisDTT, Bis-2-mercaptoethanol, Bis-DTE, Bis-Pyr, Bis-Ery, Bis-Tol, Bis-BDT, Bis-PDT, Bis-Pyr/Bal, Bis-Pyr/BDT, BisPyr/EDT, Bis-Pyr/PDT, Bis-Pyr/Tol, Bis-Pyr/Ery, bismuth-1-mercapto-2-propanol, and Bis-EDT/2-hydroxy-1-propanethiol. In some embodiments, the BT compound is BisEDT. In some embodiments, the composition is a suspension of microparticles comprising said BT compounds having a volumetric mean diameter (VMD) from about 0.4 μm to about 5 μm. In some embodiments, at least 70% of the microparticles have a volumetric mean diameter of from about 0.4 μm to about 3 μm, or from about 0.5 μm to about 2 μm, or from about 0.7 μm to about 2 μm, or from about 0.8 μm to about 1.8 μm, or from about 0.8 μm to about 1.6 μm, or from about 0.9 μm to about 1.4 μm, or from about 1.0 μm to about 2.0 μm, or from about 1.0 μm to about 1.8 μm, or any narrow ranges between the specific ranges described above.

In some embodiments of the methods for treating a microbial (e.g. bacterial and/or fungal) infection, the BT composition comprises BisEDT and the applied BisEDT is present on the surface at a concentration greater than about 20 μg/cm².

In some embodiments of the methods for treating a microbial (e.g. bacterial and/or fungal) infection, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH 7.4.

In some embodiments of the methods for treating a microbial (e.g. bacterial and/or fungal) infection such as osteomyelitis, the method comprises at least one of: (i) reducing and or dispersing a microbial (e.g. bacterial and/or fungal) biofilm, (ii) impairing growth or formation of a microbial (e.g. bacterial and/or fungal) biofilm, and (iii) preventing reformation or spread of a microbial (e.g. bacterial and/or fungal) biofilm. In some embodiments, the BT composition treats, manages or lessens the severity of the diabetic foot infection by one or both of: (i) prevention of the infection by the bacterial or fungal pathogen; and (ii) reduction of the bacterial or fungal pathogen. In some embodiments, the BT composition treats, manages or lessens the severity of the infection by one or more of: (i) prevention of elaboration or secretion of exotoxins from the bacterial or fungal pathogen; (ii) inhibition of cell viability or cell growth of planktonic cells of the bacterial or fungal pathogen; (iii) inhibition of biofilm or microbial pathogen formation by the bacterial or fungal pathogen; (iv) inhibition of biofilm invasiveness to underlying tissues (e.g. subcutaneous tissue); (v) inhibition of biofilm or microbial pathogen pathogenicity to underlying tissues (e.g. subcutaneous tissue); (vi) inhibition of biofilm viability or biofilm growth of biofilm-forming cells of the bacterial or fungal pathogen; and/or (vii) prevents the reformation of biofilm after debridement.

In some embodiments of the methods for treating a microbial (e.g. bacterial and/or fungal) infection such as osteomyelitis, the applied BT composition is present on a surface at a concentration from about 1 μg/cm² to about 1,000,000 μg/cm²(e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration from about 250 μg/cm² to about 5,000 μg/cm². For example, in some embodiments, the bismuth thiol compound in the BT composition is BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In another embodiment, the dose volume may range from about 0.01 mL to about 10 mL or any range therein between. In another embodiment, the dose volume may range from about 0.1 mL to about 1 mL or any range therein between. In another embodiment, the minimal dose volume is about 0.1 mL to about 0.5 mL or any range therein between.

In some embodiments of the methods for treating a microbial (e.g. bacterial and/or fungal) infection, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments of the methods for treating a microbial (e.g. bacterial and/or fungal) infection, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides methods treating osteomyelitis in a subject having a pre-existing condition, comprising administering the subject a therapeutically effective amount of a composition comprising BisEDT, wherein the composition is a suspension of microparticles comprising said BisEDT wherein at least 70% of the microparticles have a volumetric mean diameter (VMD) from about 0.4 μm to about 5 μm, and wherein the composition is applied to the infection (e.g. applied to the surface of the infection) and the wound is healed or substantially healed within 12 weeks of the first administration of the composition. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH 7.4. In some embodiments, the pre-existing condition is diabetes, cancer, or an autoimmune disease. In some embodiments, the subject is suffering from a diabetic foot infection. In some embodiments, the subject is taking immunosuppressive drugs or has a condition that suppresses the immune system. In some embodiments, the subject is receiving chemotherapy or other form of cancer treatment that suppresses the immune system. In some embodiments, the subject is undergoing hemodialysis.

In some embodiments of the methods for treating osteomyelitis, bone infection, or infection near a bone or orthopaedic device inserted in a subject, the BT composition is applied on the surface of an open wound or surgical site at a concentration from about 1 μg/cm² to about 1,0,00 μg/cm² (e.g. about 1 μg/cm² to about 10,000 μg/cm²). In some embodiments, the BT composition is applied at a concentration from about 50 μg/cm² to about 100 μg/cm². In some embodiments, the BT composition is applied at a concentration greater than about 100 μg/cm² (e.g. as a dosage from about 250 μg/cm² to about 5,000 μg/cm²). For example, in some embodiments, the bismuth thiol compound in the BT composition is BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments of the methods for treating osteomyelitis bone infection, or infection near a bone or orthopaedic device inserted in a subject, the BT composition is administered three times per day, two times per day, once daily, every other day, once every three days, three times per week, once every week, once every other week, once every month, or once every other month. In some embodiments, the wound is healed 4 weeks, 8 weeks or 12 weeks after the first administration of the BT composition. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, the present disclosure provides methods for reducing the risk of amputation and/or infection-related surgery in a subject having osteomyelitis, comprising administering to the subject a therapeutically effective amount of a composition comprising BisEDT, wherein the composition is applied to the infection (e.g. applied to the surface of the infection) and the risk of amputation and/or infection-related surgery is reduced from about 1% to about 100% relevant to a similarly situated subject not treated with a therapeutically effective amount of a composition comprising a bismuth-thiol compound. In some embodiments, the composition is a suspension of microparticles comprising said BisEDT wherein at least 70% of the microparticles have a volumetric mean diameter (VMD) from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH 7.4.

In some embodiments, of the methods for reducing the risk of amputation and/or infection-related surgery in a subject having osteomyelitis, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In some embodiments, of the methods for reducing the risk of amputation and/or infection-related surgery in a subject having osteomyelitis, the BT composition is administered three times per day, two times per day, once daily, every other day, once every three days, three times per week, once every week, once every other week, once every month, or once every other month. In some embodiments, the BT composition is administered once daily or three times per week. In some embodiments, the subject is administered multiple doses of the BT composition daily or weekly for a length of time ranging from about one week to about 12 weeks. In some embodiments, the subject is administered multiple doses of the BT composition daily or weekly for a length of time longer than about 12 weeks. For example, about 4 months, about 5 months, about 6 months, about 7 months, about 8 months, about 9 months, about 10 months, about 11 months, or about 1 year. In some embodiments, the subject is administered multiple doses of the BT composition daily or weekly for a length of about 4 weeks. In some embodiments, the subject is administered multiple doses of the BT composition daily or weekly for a length of about 4 weeks to about 10 weeks. In some embodiments, the pharmaceutical composition is administered every 4 hours or every 6 hours for an initial 24 hours. In some embodiments, following the initial 24 hours, the pharmaceutical composition is administered every 12 hours or every 24 hours for at least 3 additional days. In some embodiments, the pharmaceutical composition is administered every 12 hours or every 24 hours for at least 4 additional days.

In some embodiments, of the methods for reducing the risk of amputation and/or infection-related surgery in a subject having osteomyelitis, the wound area is from about 0.1 cm² to about 250 cm². For example, the wound area may be about 0.1 cm², about 0.5 cm², about 1 cm², about 5 cm², about 10 cm², about 15 cm², about 20 cm², about 25 cm², about 30 cm², about 35 cm², about 40 cm², about 45 cm², about 50 cm², about 55 cm², about 60 cm², about 65 cm², about 70 cm², about 75 cm², about 80 cm², about 85 cm², about 90 cm², about 95 cm², about 100 cm², about 105 cm², about 110 cm², about 115 cm², about 120 cm², about 125 cm², about 130 cm², about 135 cm², about 140 cm², about 145 cm², about 150 cm², about 155 cm², about 160 cm², about 165 cm², about 170 cm², about 175 cm², about 180 cm², about 185 cm², about 190 cm², about 195 cm², about 200 cm², about 205 cm², about 210 cm², about 215 cm², about 220 cm², about 225 cm², about 230 cm², about 235 cm², about 240 cm², about 245 cm², or about 250 cm².

In some embodiments, the present disclosure provides a method for preventing amputation and/or infection-related surgery in a subject having osteomyelitis, comprising administering to the subject a therapeutically effective amount of a BT composition. In some embodiments, the BT composition is a suspension of microparticles comprising said BisEDT wherein at least 70% of the microparticles have a volumetric mean diameter (VMD) from about 0.4 μm to about 5 μm. In some embodiments, the BT composition further comprises about 0.05% to about 1.0% Tween 80®, about 0.05 to 40 mM sodium chloride, optionally about 1% to about 10% of methylcellulose, and optionally about 2 to 20 mM sodium phosphate at about pH 7.4.

In some embodiments, of the methods for preventing amputation and/or infection-related surgery in a subject having osteomyelitis, the applied BT composition is present on the surface at a concentration from about 1 μg/cm² to about 1,000,000 μg/cm² (e.g. about 1 μg/cm² to about 10,000 μg/cm²). In some embodiments, the applied BT composition is present on the surface at a concentration from about 50 μg/cm² to about 100 μg/cm². In some embodiments, the applied BT composition is present on the surface at a concentration greater than about 100 μg/cm² (e.g. as a dosage from about 250 μg/cm² to about 5,000 μg/cm²). For example, in some embodiments, the bismuth thiol compound in the BT composition is BisEDT which is present on the surface at a concentration from about 1 μg/cm² to about 10,000 μg/cm² or from about 50 μg/cm² to about 200 μg/cm² or from about 250 μg/cm² to about 5,000 μg/cm². In some embodiments, the BT composition is present on the surface at a concentration of about 1 μg/cm², about 50 μg/cm², about 100 μg/cm², about 150 μg/cm², about 200 μg/cm², about 250 μg/cm², about 500 μg/cm², about 750 μg/cm², about 1000 μg/cm², about 1500 μg/cm², about 2000 μg/cm², about 2500 μg/cm², about 3000 μg/cm², about 3500 μg/cm², about 4000 μg/cm², about 4500 μg/cm², about 5000 μg/cm², about 5500 μg/cm², about 6000 μg/cm², about 6500 μg/cm², about 7000 μg/cm², about 7500 μg/cm², about 8000 μg/cm², about 8500 μg/cm², about 9000 μg/cm², about 9500 μg/cm², to about 10,000 μg/cm².

In any of the embodiments of the methods described herein, the BT composition that is ultimately applied or administered to the infected bone of a subject has a concentration from about 1 μg/mL to about 1,000,000 μg/mL (e.g. about 1 μg/cm² to about 10,000 μg/cm²). In some embodiments, the BT composition has a concentration from about 50 μg/mL to about 100 μg/mL. In some embodiments, the applied BT the BT composition has a concentration from about 250 μg/mL to about 5,000 μg/mL. For example, in some embodiments, the bismuth thiol compound in the BT composition is BisEDT which has a concentration from about 1 μg/mL to about 10,000 μg/mL or from about 50 μg/mL to about 200 μg/mL or from about 250 μg/mL to about 5,000 μg/mL. In some embodiments, the BT composition has a concentration of about 1 μg/mL, about 50 μg/mL, about 100 μg/mL, about 150 μg/mL, about 200 μg/mL, about 250 μg/mL, about 500 μg/mL, about 750 μg/mL, about 1000 μg/mL, about 1500 μg/mL, about 2000 μg/mL, about 2500 μg/mL, about 3000 μg/mL, about 3500 μg/mL, about 4000 μg/mL, about 4500 μg/mL, about 5000 μg/mL, about 5500 μg/mL, about 6000 μg/mL, about 6500 μg/mL, about 7000 μg/mL, about 7500 μg/mL, about 8000 μg/mL, about 8500 μg/mL, about 9000 μg/mL, about 9500 μg/mL, to about 10,000 μg/mL. In some embodiments of the methods described herein, the BT composition that is ultimately applied or administered to the subject has a concentration greater than about 1,000,000 μg/mL.

In some embodiments of the methods described herein, the pharmaceutical composition is a bismuth-thiol (BT) composition that comprises BisEDT suspended therein, wherein the BT composition comprises a plurality of microparticles. In a specific embodiment, the D90 of said microparticles is less than or equal to 4.5 μm, or 4.0 μm, or 3.5 μm, or 3.0 μm, or 2.5 μm, or 2.0 μm, or 1.9 μm, or 1.8 μm, or μm 1.7 μm, or 1.6 μm, or 1.5 μm or any ranges in between. In a specific embodiment, the D90 of said microparticles is less than or equal to 1.9 μm. In another specific embodiment, the D90 of said microparticles is less than or equal to 1.6 μm. In another specific embodiment, the D50 of said microparticles is less than or equal to 2.5 μm, or 2.0 μm, or 1.5 μm, or 1.3 μm, or 1.2 μm, or 1.1 μm, or 1.0 μm, or 0.9 μm, or 0.87 μm, or 0.72 μm or any ranges in between. In another specific embodiment, the D10 of said microparticles is less than or equal to 0.9 μm, or 0.8 μm, or 0.7 μm, or 0.6 μm, or 0.50 μm, or 0.40 μm, or 0.39 μm, or 0.38 μm, or 0.37 μm, or 0.36 μm, or 0.35 μm, or 0.34 μm, or 0.33 μm, or any ranges in between. In a specific embodiment, the pharmaceutical composition comprising bismuth-thiol (BT) composition comprises BisEDT suspended therein, wherein the BT composition comprises a plurality of microparticles, wherein the D90 of said microparticles is less than or equal to about 1.6 μm. In a specific embodiment, the BT composition comprises about 0.05% to about 1.0% Tween 80®, about 0.05 to 40 mM sodium chloride, optionally about 1% to about 10% of methylcellulose, and optionally about 2 to 20 mM sodium phosphate at about pH. 7. In another specific embodiment, the compositions described above can be administered to a subject for treating osteomyelitis in a subject, or any specific methods described herein.

### EXAMPLES

The following examples are provided to illustrate the present disclosure, and should not be construed as limiting thereof. Additional experimental procedures and details can be found in International Patent Application Nos. PCT/US2010/023108, PCT/US2011/023549, and PCT/US2011/047490, which are hereby incorporated by reference in their entireties for all purposes.

### Example 1: General Synthesis of BisEDT

The starting materials and reagents used in preparing these compounds are either available from commercial supplier such as Aldrich Chemical Co., Bachem, etc., or can be made by methods well known in the art. The starting materials and the intermediates and the final products of the reaction can be isolated and purified if desired using conventional techniques, including but not limited to filtration, distillation, crystallization, chromatography, and the like and can be characterized using conventional means, including physical constants and spectral data. Unless specified otherwise, the reactions described herein take place at atmospheric pressure over a temperature range from about −78° C. to about 150° C.

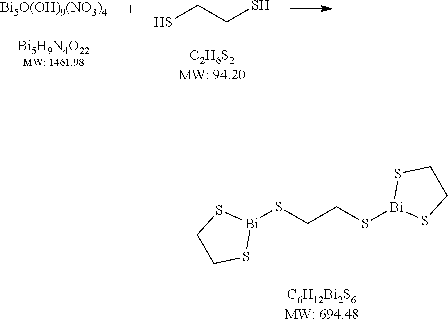

Microparticulate bismuth-1,2-ethanedithiol (Bis-EDT, soluble bismuth preparation) was prepared as follows: To an excess (11.4 L) of 5% aqueous HNO₃ at room temperature in a 15 L polypropylene carboy was slowly added by dropwise addition 0.331 L (˜0.575 moles) of an aqueous Bi(NO₃)₃ solution (43% Bi(NO₃)₃ (w/w), 5% nitric acid (w/w), 52% water (w/w), Shepherd Chemical Co., Cincinnati, OH, product no. 2362; δ˜1.6 g/mL) with stirring, followed by slow addition of absolute ethanol (4 L). Some white precipitate formed but was dissolved by continued stirring. An ethanolic solution (˜1.56 L, ˜0.55 M) of 1,2-ethanedithiol (CAS 540-63-6) was separately prepared by adding, to 1.5 L of absolute ethanol, 72.19 mL (0.863 moles) of 1,2-ethanedithiol using a 60 mL syringe, and then stirring for five minutes. The 1,2-ethanedithiol/EtOH reagent was then slowly added by dropwise addition over the course of five hours to the aqueous Bi(NO₃)₃/HNO₃ solution, with continued stirring overnight. The formed product was allowed to settle as a precipitate for approximately 15 minutes, after which the filtrate was removed at 300 mL/min using a peristaltic pump. The product was then collected by filtration on fine filter paper in a 15-cm diameter Buchner funnel, and washed sequentially with three, 500-mL volumes each of ethanol, USP water, and acetone to obtain BisEDT (694.51 gm/mole) as a yellow amorphous powdered solid. The product was placed in a 500 mL amber glass bottle and dried over CaCl₂) under high vacuum for 48 hours. Recovered material (yield˜200 g) gave off a thiol-characteristic odor. The crude product was redissolved in 750 mL of absolute ethanol, stirred for 30 min, then filtered and washed sequentially with 3×50 mL ethanol, 2×50 mL acetone, and washed again with 500 mL of acetone. The rewashed powder was triturated in 1M NaOH (500 mL), filtered and washed with 3×220 mL water, 2×50 mL ethanol, and 1×400 mL acetone to afford 156.74 gm of purified BisEDT. Subsequent batches prepared in essentially the same manner resulted in yields of about 78-91%.

The product was characterized as having the structure shown above by analysis of data from ¹H and ¹³C nuclear magnetic resonance (NMR), infrared spectroscopy (IR), ultraviolet spectroscopy (UV), mass spectrometry (MS) and elemental analysis. An HPLC method was developed to determine chemical purity of BisEDT whereby the sample was prepared in DMSO (0.5 mg/mL). The λₘₐₓ was determined by scanning a solution of BisEDT in DMSO between 190 and 600 nm. Isocratic HPLC elution at 1 mL/min was performed at ambient temperature in a mobile phase of 0.1% formic acid in acetonitrile:water (9:1) on a Waters (Millipore Corp., Milford, MA) model 2695 chromatograph with UV detector monitoring at 265 nm (λₘₐₓ), 2 μL injection volume, equipped with a YMC Pack PVC Sil NP, 5 μm, 250×4.6 mm inner diameter analytical column (Waters) and a single peak was detected, reflecting chemical purity of 100±0.1%. Elemental analysis was consistent with the structure of BisEDT as shown above. The dried particulate matter was characterized to assess the particle size properties. Briefly, microparticles were resuspended in 2% Pluronic® F-68 (BASF, Mt. Olive, NJ) and the suspension was sonicated for 10 minutes in a water bath sonicator at standard setting prior to analysis using a Nanosizer/Zetasizer Nano-S particle analyzer (model ZEN1600 (without zeta-potential measuring capacity), Malvern Instruments, Worcestershire, UK) according to the manufacturer's recommendations. From compiled data of two measurements, microparticles exhibited a unimodal distribution with all detectable events between about 0.6 microns and 4 microns in volumetric mean diameter (VMD) and having a peak VMD at about 1.3 microns.

### Example 2: Preparation of Microparticulate Bismuth-1-2-ethanedithiol (BisEDT

Microparticulate bismuth-1,2-ethanedithiol (Bis-EDT) was prepared as follows: Water (25.5 L) and 70% nitric acid (1800 mL) were mixed together in a Nalgene reactor. Then, water (2300 mL) was added to an Erlenmeyer flask, followed by bismuth subnitrate (532 g), and the mixture was stirred. To the mixture was added 70% nitric acid (750 mL) to obtain a clear solution. This solution was transferred into the Nalgene reactor and the resulting mixture was stirred for 20 min. Then, 9.5 L of 95% EtOH was added to the reactor in three portions.

Separately, 1,2-ethanedithiol, 98%, (229 mL) was added to a bottle followed by two 250 mL EtOH portions with stirring. A further 5 L EtOH was added to the bottle with stirring. The 1,2-ethanedithiol solution was then added to the reactor over about 4 hours while stirring. After stirring for 18 hours, the solids were allowed to settle for 2 hours. EtOH (20 L) was added and the mixture stirred for 24 hours. The solids were allowed to settle for 1.5 hours, then separated by filtration of the mixture, followed by rinsing with EtOH.

To the empty reactor was added 9 L EtOH and the filtered solids, which was stirred for 18 hours. The solids were allowed to settle for 1 hour, then separated by filtration of the mixture, followed by rinsing with EtOH. Next, the empty reactor was charged with 9 L acetone, 99.5%, and the filtered solids, which was stirred for 15 hours. The solids were allowed to settle for 1.5 hours, then separated by filtration of the mixture, followed by rinsing with acetone. Again, the empty reactor was charged with 9 L acetone, 99.5%, and the filtered solids, which was stirred for 1.4 hours. The solids were filtered and air-dried for 69 hours, then vacuum-dried for 4 hours. After mixing the solid, it was sieved through a 10 mesh (2 mm) and then 18 mesh (1 mm) sieve to give BisEDT.

### Example 3: Synthesis of Additional Bismuth Thiol Compounds

The following bismuth thiol compounds can also be prepared according to the methods of Examples 1 and 2:

bismuth-2,3-dimercaptopropanol (2:3 molar ratio, BisBAL, MB-2B)
bismuth-4-methyl-1,2-benzenedithiol (2:3 molar ratio, BisTOL; MB-5)
bismuth-2,3-butanedithiol (BisBDT, MB-6)

### Example 4: Preparation of Microparticulate bismuth-1,2-ethanedithiol (BisEDT

### Studies on Processing Conditions on BisEDT Particle Size Distribution

It was observed that careful control of the reaction temperature and the rate of 1,2 ethanedithiol addition had pronounced impact on the BisEDT particle size distribution. Representative syntheses are shown below for BisEDT synthesized at 20° C. with a 1.25 hour addition of 1,2-ethane via syringe pump and BisEDT synthesized at 15° C. with a 1 hour addition of 1,2-ethane via syringe pump. Table 1 below shows that temperature conditions play a critical role in particle size distribution, with processing temperatures in the range of 20-30° C. providing BisEDT particle size distribution that are both small and uniform in particle size (such as a D90 below 2 microns).

Representative synthesis of BisEDT at 20° C. with 1.25 hour addition of thiol via syringe pump, and polypropylene cloth for filtration BisEDT synthesis was performed on 10-g scale. To a 1-L jacketed reactor was charged USP water (480 mL, 48 vol), followed by 70% HNO3 (34 mL, 3.4 vol). A solution of bismuth subnitrate (10 g, 6.84 mmols) in water (43 mL, 4.3 vol) and 70% HNO₃ (14 mL, 1.4 vol) was added at 20° C. The reaction mixture was cooled to 15° C. for addition of 95% Ethanol. The 95% ethanol (180 mL, 18 vol) was then added slowly. (Ethanol addition is exothermic, temperature reached 22° C.). The temperature was then adjusted back to 20° C. This was followed by dropwise addition of 1,2 ethanedithiol (4.3 mL, 7.5 mmols in 95% ethanol in 94 mL, 9.4 vol) over a period of 1.25 hour with the batch temperature at 20° C. during which time it turned into a yellow suspension. The reaction was stirred at 20° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.81 g 91.0%).

Representative synthesis of BisEDT at 15° C. with 1 hour addition of thiol via syringe pump, and polypropylene cloth for filtration: The synthesis BisEDT was performed on 10-g scale, temperature profile was studied with data logger. Ethane-dithiol was added at 15° C. over 1 hour via syringe pump and the filtration was performed using PP filter cloth. To a 1-L jacketed reactor was charged USP water (480 mL, 48 vol) and cooled to 15° C., followed by 70% HNO3 (34 mL, 3.4 vol). A solution of bismuth subnitrate (10 g, 6.84 mmols) in water (43 mL, 4.3 vol) and 70% HNO₃ (14 mL, 1.4 vol) was added at the same temperature. The 95% ethanol (180 mL, 18 vol) was then added slowly (Ethanol addition is exothermic, temperature reached 22.5° C.). It was then allowed to cool to 15° C. This was followed by dropwise addition of 1,2-ethanedithiol (4.3 mL, 7.5 mmols in 95% ethanol in 94 mL, 9.4 vol) over an hour with the batch temperature at 15° C. The reaction was allowed to stir at 15° C. overnight. The reaction mixture was filtered through polypropylene cloth and washed with 95% ethanol (45 mL, 4.5 vol). The wet cake was charged back to the reactor and slurried in 95% ethanol (380 mL, 38 vol) for two hours at 20° C. The suspension was then filtered (same cloth) and washed with 95% ethanol (30 mL, 3 vol). The wet cake was again slurried in 95% EtOH (170 mL, 17 vol) at 20° C., filtered (same cloth), and washed with 95% ethanol (30 mL, 3 vol). The wet cake was then slurried in acetone (170 mL, 17 vol) at 20° C. overnight, followed by filtration (same cloth) and acetone wash (20 mL, 2 vol). The acetone (170 ml, 17 vol) treatment was repeated on the solids and stirred for 2 hours. The suspension was filtered (same cloth) and washed with acetone (30 mL, 3 vol) and died at 45° C. and dried at 45° C. (18 hours) to provide canary yellow solid (10.43 g 87.8%).

TABLE 1
Particle Size Distribution of BisEDT samples prepared by improved method

| Sample | D(10) μm | D(50) μm | D(90) μm | D[4,3] μm | Conditions |
|---|---|---|---|---|---|
| 1 | 0.80 | 2.4 | 5.9 | 2.9 | Dalton Synthesis Conditions |
| 2 | 0.58 | 1.7 | 3.9 | 2.0 | Dalton Synthesis Conditions |
| 3 | 0.59 | 1.9 | 4.5 | 2.3 | 30° C., 5 h addition of 1,2- ethane dithiol |
| | | | | | via addition funnel |
| 4 | 0.44 | 1.48 | 3.1 | 1.7 | 30° C., 4 hour addition of 1,2-ethane |
| | | | | | dithiol via syringe pump |
| 5 | 0.33 | 0.72 | 1.6 | 0.86 | 20° C., 1 h addition of 1,2- ethane dithiol |
| | | | | | via addition funnel |
| 6 | 0.34 | 0.87 | 1.8 | 0.98 | 20° C., 4 h addition of 1,2- ethane dithiol |
| | | | | | via addition funnel |
| 7 | 0.39 | 1.3 | 1.6 | 1.4 | 20° C., 1 hour addition of 1,2-ethane |
| | | | | | dithiol via syringe pump. Sample |
| | | | | | slurried in EtOH. Cloth filtration |
| 8 | 0.36 | 1.0 | 1.8 | 1.0 | 20° C., 1 hour addition of 1,2-ethane |
| | | | | | dithiol via syringe pump. Sample |
| | | | | | slurried in MeOH. Cloth filtration |
| 9 | 0.67 | 1.0 | 1.9 | 1.1 | 20° C., 1 hour addition of 1,2-ethane |
| | | | | | dithiol via syringe pump. Sample |
| | | | | | slurried in Abs. MeOH. Cloth filtration |
| 10 | 0.36 | 0.88 | 1.7 | 0.97 | 20° C., 1 hour addition of 1,2-ethane |
| | | | | | dithiol via syringe pump. Sample |
| | | | | | slurried in IPA. Cloth filtration |
| 11 | 0.38 | 1.2 | 2.4 | 1.4 | 15° C. 1.5 hour addition of 1,2-ethane |
| | | | | | dithiol via syringe pump. Cloth filtration |
| 12 | 0.37 | 1.2 | 2.4 | 1.3 | 20° C., 1.25 hour addition of 1,2-ethane |
| | | | | | via syringe pump |
| 13 | 0.36 | 0.98 | 2.1 | 1.2 | 10° C., 1 h addition of 1,2- ethane dithiol |
| | | | | | via syringe pump |
| 14 | 0.36 | 1.0 | 2.1 | 1.2 | 10° C. 1 hour addition of 1,2-ethane dithiol |
| | | | | | via syringe pump. Cloth filtration |
| 15 | 0.32 | 0.72 | 1.6 | 0.86 | 10° C., 4 hours addition of 1,2-ethane |
| | | | | | dithiol via syringe pump. Cloth filtration. |

### Example 5: In Vitro Activity of Bismuth Thiols Against Drug Resistant Gram-Positive and Gram-Negative Bacteria and Yeast

Introduction: The in vitro activity of three Bismuth Thiol compounds were evaluated against organisms currently identified by the Centers for Disease Control (CDC; 1) as top drug-resistant threats in the United States, including ESKAPE pathogens (2, 3), C. difficile, resistant gonococci, and azole-resistant Candida spp. The susceptibility of test isolates to the Bismuth Thiol compounds MB-1-B3 (BisEDT), MB-2B (BisBAL), and MB-6 (BisBDT) and relevant comparators was evaluated in accordance with guidelines from the Clinical and Laboratory Standards Institute (CLSI; 4-8).

### Materials and Methods

Test and Comparator Agents: The test agents were stored at room temperature until assayed. All test agents were suspended and diluted in 100% dimethylsulfoxide (DMSO), and were ultimately tested at a final concentration of 0.06-64 μg/mL. The stock solutions were allowed to stand for at least 1 hr prior to use to auto-sterilize.

Comparator drugs were tested over a concentration range spanning established quality control ranges and breakpoints. Information on comparator compounds used during testing are described in Table 2 below:

TABLE 2
Comparator Compounds

| | Manufac- | | | |
|---|---|---|---|---|
| Drug | turer | Lot No. | Solvent | Diluent |
| Levofloxacin | Sigma | BCBC2112V | 0.1M NaOH | Water |
| Meropenem | USP | IOJ244 | Water | Water |
| Ceftazidime | USP | L1K237 | Water | Water |
| Gentamicin | Sigma | SLBM9736V | Water | Water |
| Vancomycin | | | Water | Water |
| Penicillin | Sigma | 071M0740V | Water | Water |
| Oxacillin | Sigma | BCBF5635V | Water | Water |
| Clindamycin | Sigma | 021M1533V | Water | Water |
| Erythromycin | Sigma | 011M1510V | 95% | Water |
| | | | ethanol | |
| Ciprofloxacin | USP | 1134335 | Water | Water |
| Metronidazole | Sigma | 095K0693 | DMSO | Water |
| Fidaxomicin | Merck | SE-B13-01- | DMSO | Water |
| | | 001885 | | |
| Fluconazole | USP | H1L308 | DMSO | DMSO |
| Amphotericin B | Sigma | 063M4043V | DMSO | DMSO |
| Trimethoprim | Sigma | 080M4044 | Water | Water |
| Sulfamethoxazole | Fluka | BCBC7096V | Water and | Water |
| | | | 2.5M NaOH | |
| | | | dropwise | |
| Ceftriaxone | USP | J1L040 | Water | Water |

Test Organisms: The test organisms consisted of reference strains from the American Type Culture Collection (ATCC; Manassas, VA) or clinical isolates from the MMX repository. The spectrum of organisms evaluated and their corresponding phenotypic information is shown in Tables 89-95. Relevant quality control organisms were included on each day of testing as specified by CLSI (4-8). The isolates were sub-cultured onto an appropriate agar medium prior to testing.

TestMedia: Test media were prepared and stored in accordance with guidelines from CLSI (4, 6, 7). Broth microdilution susceptibility testing of aerobic bacteria was performed using cation adjusted Mueller-Hinton Broth (CAMHB; Becton Dickinson [BD], Sparks, MD; Lot No. 6117994) with the exception of streptococci where CAMHB was supplemented with 5% (v/v) lysed horse blood (Cleveland Scientific, Bath, OH; Lot No. 322799). Neisseria gonorrhoeae were evaluated by agar dilution using agar consisting of GC medium base (BD; Lot No. 4274618) supplemented with 1% IsoVitaleX (BD; Lot No. 5246843).

The susceptibility of anaerobic bacteria was determined by agar dilution using Brucella Agar (BD/BBL; Lot No. 5237692) supplemented with 5 μg/mL hemin (Sigma, St. Louis, MO; Lot No. 108K1088), 1 μg/mL Vitamin K1 and 5% (v/v) laked sheep blood (Cleveland Scientific, Bath, OH; Lot No. 322799).

The susceptibility of yeast isolates was determined by broth microdilution in RPMI medium (HyClone Laboratories, Logan, UT; Lot No. AZC184041B) buffered with 0.165 M MOPS (Calbiochem, Billerica, MA; Lot No. 2694962). The pH of the medium was adjusted to 7.0 with 1 N NaOH, sterile filtered using a 0.2 μm PES filter, and stored at 4° C. until used.

Broth Microdilution MC Testing (Aerobic Bacteria and Yeast): The broth microdilution assay method employed for the susceptibility testing of aerobic bacteria (excluding N. gonorrhoeae which was evaluated by agar dilution) and yeast essentially followed the procedures described by CLSI (3, 4, 7, 8) and employed automated liquid handlers to conduct serial dilutions and liquid transfers. Automated liquid handlers included the Multidrop 384 (Labsystems, Helsinki, Finland) and Biomek 2000 (Beckman Coulter, Fullerton CA). The wells in columns 2-12 in standard 96-well microdilution plates (Costar 3795) were filled with 150 μl of the correct diluent. These would become the ‘mother plates’ from which ‘daughter’ or test plates would be prepared. The drugs (300 μL at 40× the desired top concentration in the test plates) were dispensed into the appropriate well in Column 1 of the mother plates. The Biomek 2000 was used to make serial two-fold dilutions through Column 11 in the “mother plate”. The wells of Column 12 contained no drug and ultimately served as the organism growth control wells.

The daughter plates were loaded with 185 μL per well of the appropriate test media using the Multidrop 384. The daughter plates were prepared using the Biomek FX which transferred 5 μL of drug solution from each well of a mother plate to the corresponding well of the correct daughter plate in a single step.

A standardized inoculum of each organism was prepared per CLSI methods (3, 7). Isolated colonies of each test isolate were picked from the primary plate and a suspension was prepared to equal a 0.5 McFarland turbidity standard. Standardized suspensions were then diluted 1:100 in test media (1:100 for yeast, 1:20 for bacteria). After dilution, the inoculum suspensions were then transferred to compartments of sterile reservoirs divided by length (Beckman Coulter), and the Biomek 2000 was used to inoculate all plates. Daughter plates were placed on the Biomek 2000 in reverse orientation so that plates were inoculated from low to high drug concentration.

The Biomek 2000 delivered 10 μL of standardized inoculum into each well of the appropriate daughter plate for an additional 1:20 dilution. The wells of the daughter plates ultimately contained 185 μL of the appropriate media, 5 μL of drug solution, and 10 μL of inoculum which corresponded to a final inoculum concentration of 0.5-2.5×103 CFU/mL of yeast and approximately 5×10⁵ CFU/mL of bacteria per test well. The final concentration of DMSO (if used as a solvent) in the test well was 2.5%.

Plates were stacked 4 high, covered with a lid on the top plate, placed into plastic bags, and incubated at 35° C. for approximately 24-48 hr for all yeast isolates and 16-24 hr for aerobic bacteria. Plates were viewed from the bottom using a plate viewer. An un-inoculated solubility control plate was observed for evidence of drug precipitation. MIC endpoints for the test agents and control compounds were read per CLSI criteria (3, 7).

Agar Dilution MIC Testing (Anaerobic Bacteria and Gonococci): MIC values for anaerobic bacteria were determined using a reference agar dilution method as described by CLSI (6). Organisms were grown at 35° C. in the Bactron II Anaerobic Chamber (Shel Lab, Cornelius, OR) for approximately 48 hr prior to the assay. Drug dilutions and drug-supplemented agar plates were prepared manually per CLSI (6). The plates were allowed to stand at room temperature for 1 hr to allow the agar surface to dry and pre-reduced for approximately 1 hr in the anaerobe chamber prior to inoculation. Each isolate was suspended to the equivalent of a 0.5 McFarland standard in Brucella broth using a turbidity meter (Dade Behring MicroScan, West Sacramento, CA). Each bacterial cell suspension was then diluted 1:10 in Brucella broth and transferred to wells in a stainless steel replicator block which was used to inoculate the test plates. The prongs on the replicator deliver approximately 1-2 μl of inoculum to an agar surface. The resulting inoculum spots contained approximately 1×10⁵ CFU/spot. After the inoculum dried, the inoculated drug-supplemented agar plates and no drug growth control plates were incubated at 35° C. for 42-48 hr in the anaerobe chamber. The MIC was read per CLSI guidelines (6).

MIC values for N. gonorrhoeae were determined using the reference agar dilution method as described by CLSI (4). This method followed the same agar dilution method described above for anaerobes with the exception that agar plates contained GC medium base supplemented with 1% IsoVitaleX and, after inoculation, plates were incubated aerobically at 35° C. in 5% CO2 for 20-24 hr.

### Results and Discussion

The activity of the Bismuth Thiol test agents MB-1-B3, MB-2B, and MB-6 and comparators are shown below for Enterobacteriaceae (Table 3), Pseudomonas aeruginosa and Acinetobacter baumannii (Table 4), Staphylococcus aureus and Enterococcus spp. (Table 5), streptococci (Table 6), N. gonorrhoeae (Table 7), anaerobes (Table 8), and Candida spp. (Table 9). Across all evaluated organisms, MIC results for comparator agents were within the established CLSI QC ranges for the relevant ATCC QC isolate (5, 8).

The evaluated Enterobacteriaceae (Table 1) consisted of the Escherichia coli ATCC QC isolate, ESBL-positive E. coli and Klebsiella pneumoniae, KPC-positive K. pneumoniae, and NDM-1 positive E. coli, K. pneumoniae, and Enterobacter cloacae. Excluding the ATCC QC isolate of E. coli which was susceptible, the activity of the comparators illustrates the drug-resistant nature of these isolates. Regardless of the high degree of drug resistance, the evaluated Bismuth Thiol test agents had consistent activity across all isolates. MB-1-B3 (MIC values of 0.5-4 μg/mL) and MB-6 (MIC values of 0.5-2 μg/mL) had similar activity and this activity was typically 2- to 16-fold lower than that observed with MB-2B (MIC values of 2-32 μg/mL).

The evaluated P. aeruginosa and A. baumannii (Table 4) consisted of the susceptible P. aeruginosa ATCC QC isolate, and various isolates with either metallo-beta-lactamases or multi-drug resistance. Excluding the QC isolate, the activity of comparators illustrates the drug-resistant nature of these isolates. Regardless of the high degree of drug resistance, the evaluated Bismuth Thiol test agents had consistent activity across all isolates. MB-1-B3 and MB-6 (MIC values of 0.5-2 μg/mL) had similar activity and this activity was typically 2- to 32-fold lower than that observed with MB-2B (MIC values of 2-16 μg/mL).

Against S. aureus and Enterococcus spp. (Table 5), all 3 Bismuth Thiol test compounds had potent activity regardless of resistance phenotype (MRSA for S. aureus and VRE for E. faecalis and E. faecium). The evaluated MRSA were largely susceptible to vancomycin and gentamicin but resistant to the remaining comparators. Regardless of resistance, MB-1-B3 and MB-6 had MIC values of <0.06 μg/mL against MRSA and MB-2B also had MIC values of <0.06 μg/mL with the exception of the QC isolate and MRSA MMX 9203 (MIC values of 0.5 and 0.25 μg/mL, respectively). Against enterococci, there was little activity observed with the evaluated comparators. The Bismuth Thiol test agents were active though with slightly higher MIC values for vancomycin-resistant E. faecium relative to vancomycin-resistant E. faecalis. As with the Gram-negative aerobic isolates, for enterococci MB-1-B3 (MIC values of 0.25-2 μg/mL) and MB-6 (MIC values of 0.25-1 μg/mL) had similar activity and this activity was typically 4- to 8-fold lower than that observed with MB-2B (MIC values of 2-4 μg/mL).

The evaluated streptococci (Table 6) consisted of the susceptible S. pneumoniae QC isolate, multi-drug resistant pneumococci, macrolide-resistant S. pyogenes, and clindamycin-resistant S. agalactiae. Regardless of drug-resistance phenotype, the bismuth-thiol test agents maintained activity against streptococci. Among the 3 Bismuth Thiol test agents, there was trend towards slightly higher MIC values against pneumococci relative to beta-hemolytic streptococci for MB-1-B3 and MB-6. Against pneumococci, MB-1-B3 (MIC values of 0.5-1 μg/mL) and MB-6 (MIC values of 0.5-8 μg/mL) had similar activity and this activity was typically 8- to 16-fold lower than that observed with MB-2B (MIC values of 1-8 μg/mL). Against beta-hemolytic streptococci, MB-1-B3 (MIC values of 0.03-1 μg/mL) and MB-6 (MIC values of 0.03-2 μg/mL) had similar activity and this activity was typically 4- to 16-fold lower than that observed with MB-2B (MIC values of 0.25-8 μg/mL).

As shown in Table 7, the Bismuth Thiol test agents had potent activity against the susceptible QC isolate of N. gonorrhoeae, the 3 ciprofloxacin-resistant isolates, and the single ceftriaxone non-susceptible isolate. Similar activity was observed with MB-1-B3 (MIC values of 0.06-0.12 μg/mL) and MB-6 (MIC values of 0.06-0.25 μg/mL) and this activity was slightly greater than that observed for MB-2B (MIC values of 0.12-0.5 μg/mL).

Against the evaluated anaerobes (Table 8) which consisted of the susceptible Bacteroides fragilis and Clostridium difficile QC isolates and C. difficile with various clinically important ribotypes including 027 (hypervirulent strain), MB-1-B3 (MIC values of 0.25-2 μg/mL) and MB-6 (MIC values of 1-4 μg/mL) had similar activity and this activity was typically slightly greater than that observed with MB-2B (MIC values of 2-16 μg/mL). Resistance to comparators clindamycin, metronidazole, and fidaxomicin appeared to have no impact of the activity of the Bismuth Thiol test agents.

Finally, against azole-resistant isolates of clinically prevalent Candida spp. (Table 9) including C. parapsilosis, C. albicans, C. glabrata, and C. tropicalis, the Bismuth Thiol test agents were active. A trend towards higher MIC values for the test agents was observed with C. albicans and C. tropicalis relative to C. parapsilosis and C. glabrata. All 3 Bismuth Thiol test agents had similar activity against yeast, with MIC values of 0.25-0.5 μg/mL at 24 hr against C. parapsilosis and C. glabrata, 1-4 μg/mL against C. albicans, and 1-16 μg/mL against C. tropicalis.

In summary, the broad spectrum activity of the Bismuth Thiol test agents evaluated in this study was clear and the activity observed against susceptible QC isolates was maintained against drug resistant isolates regardless of the organism or resistance phenotype evaluated. The Bismuth Thiol test agents were the most active against MRSA, N. gonorrhoeae, and beta-hemolytic streptococci based on MIC values but were also highly active against Gram-negative aerobes, S. pneumoniae, C. difficile, and yeast. In general, test agents MB-1-B3 and MB-6 had similar activity by MIC and both were more potent than MB-2B, with the exception of yeast and to a lesser extent N. gonorrhoeae where all 3 compounds had similar activity profiles.

TABLE 3
Minimal Inhibitory Concentration (MIC) Values for Microbion Bismuth Thiol Test Agents and Comparators Against Enterobacteriaceae

| | | MIC (μg/mL) | | | | | | | |
|---|---|---|---|---|---|---|---|---|---|
| Isolate | Type | MB-1-B3 | MB-2B | MB-6 | Levofloxacin | Ciprofloxacin | Meropenem | Ceftazidime | Gentamicin |
| E. coli | QC | 0.5 | 2 | 0.5 | 0.015 | 0.008 | 0.03 | 0.12 | 1 |
| MMX 102 | | | | | (0.008-0.06)¹ | (0.004-0.015) | (0.008-0.06) | (0.06-0.5) | (0.25-1) |
| (ATCC 25922) | | | | | | | | | |
| E. coli | ESBL | 1 | 2 | 1 | 16 | 32 | 0.03 | 32 | 64 |
| MMX 8423 | Lev^{R} CAZ^{R} | | | | | | | | |
| | Gm^{R} | | | | | | | | |
| E. coli | ESBL | 0.5 | 4 | 0.5 | 0.06 | 0.015 | 0.03 | 16 | 0.25 |
| MMX 8424 | CAZ^{R} | | | | | | | | |
| E. coli | ESBL | 1 | 2 | 1 | 16 | >64 | 0.015 | 16 | >64 |
| MMX 8425 | Lev^{R} CAZ^{R} | | | | | | | | |
| | Gm^{R} | | | | | | | | |
| E. coli | NDM-1 | 1 | 2 | 1 | 16 | >64 | 32 | >32 | >64 |
| MMX 5980 (ATCC | Lev^{R} | | | | | | | | |
| BAA-2469) | MEM^{R} | | | | | | | | |
| | CAZ^{R} | | | | | | | | |
| | Gm^{R} | | | | | | | | |
| K. pneumoniae | KPC-2 | 1 | 8 | 1 | >64 | >64 | 32 | >32 | 1 |
| MMX 4683 | Lev^{R} | | | | | | | | |
| | MEM^{R} | | | | | | | | |
| | CAZ^{R} | | | | | | | | |
| K. pneumoniae | KPC-2 | 2 | 16 | 1 | 1 | 0.03 | >64 | >32 | 0.25 |
| MMX 4622 | MEM^{R} | | | | | | | | |
| | CAZ^{R} | | | | | | | | |
| K. pneumoniae | KPC-2 | 2 | 32 | 0.5 | 1 | 2 | >64 | >32 | 64 |
| MMX 4623 | MEM^{R} | | | | | | | | |
| | CAZ^{R} | | | | | | | | |
| | Gm^{R} | | | | | | | | |
| K. pneumoniae | KPC-3 | 2 | 8 | 1 | 64 | >64 | 32 | >32 | 8 |
| MMX 4694 | Lev^{R} | | | | | | | | |
| | MEM^{R} | | | | | | | | |
| | CAZ^{R} | | | | | | | | |
| K. pneumoniae | KPC-3 | 4 | 16 | 2 | 64 | >64 | >64 | >32 | 1 |
| MMX 4653 | Lev^{R} | | | | | | | | |
| | MEM^{R} | | | | | | | | |
| | CAZ^{R} | | | | | | | | |
| K. pneumoniae | ESBL | 4 | 32 | 2 | 0.03 | 0.5 | 8 | 8 | 0.25 |
| MMX 4684 | MEM^{R} | | | | | | | | |
| K. pneumoniae | ESBL | 2 | 8 | 1 | 32 | 64 | 4 | >32 | 1 |
| MMX 4685 | Lev^{R} | | | | | | | | |
| | MEM^{R} | | | | | | | | |
| | CAZ^{R} | | | | | | | | |
| K. pneumoniae | NDM-1 | 2 | 16 | 1 | >64 | >64 | >64 | >32 | >64 |
| MMX 5979 | Ley^{R} | | | | | | | | |
| | MEM^{R} | | | | | | | | |
| | CAZ^{R} | | | | | | | | |
| | Gm^{R} | | | | | | | | |
| E. cloacae | NDM-1 | 4 | 32 | 2 | 64 | >64 | >64 | >32 | >64 |
| MMX 5981 | Lev^{R} | | | | | | | | |
| (ATCC BAA-2468) | MEM^{R} | | | | | | | | |
| | CAZ^{R} | | | | | | | | |
| | Gm^{R} | | | | | | | | |

QC = quality control; ESBL = extended-spectrum beta-lactamase; KPC = K. pneumoniae carbapenemase; NDM = New Delhi Metallo-beta-lactamase; Lev^{R} = Levofloxacin-resistant; MEM^{R} = Meropenem-resistant; CAZ^{R} = ceftazidime-resistant; Gm^{R} = Gentamicin-resistant
¹CLSI QC ranges shown in parenthesis where applicable

TABLE 4
Minimal Inhibitory Concentration (MIC) Values for Microbion Bismuth Thiol
Test Agents and Comparators Against P. aeruginosa and A. baumannii

| | | MI | | | | | | | |
|---|---|---|---|---|---|---|---|---|---|
| Isolate | Type | MB-1-B3 | MB-2B | MB-6 | Levofloxacin | Ciprofloxacin | Meropenem | Ceftazidime | Gentamicin |
| P. aeruginosa | QC | 1 | 8 | 1 | 1 | 0.5 | 0.5 | 2 | 1 |
| MMX 103 | | | | | (0.5-4) | (0.25-1) | (0.25-1) | (1-4) | (0.5-2) |
| (ATCC 27853) | | | | | | | | | |
| P. aeruginosa | VIM-2 | 1 | 16 | 2 | 32 | 32 | 8 | 32 | 4 |
| MMX 4697 | Lev^{R} | | | | | | | | |
| | Cip^{R} | | | | | | | | |
| | MEM^{R} | | | | | | | | |
| | CAZ^{R} | | | | | | | | |
| P. aeruginosa | IMP-7 | 2 | 8 | 2 | 64 | 32 | >64 | >32 | >64 |
| MMX 4654 | Lev^{R} | | | | | | | | |
| | Cip^{R} | | | | | | | | |
| | MEM^{R} | | | | | | | | |
| | CAZ^{R} | | | | | | | | |
| P. aeruginosa | MDR | 1 | 4 | 2 | 64 | 64 | 32 | >32 | 8 |
| MMX 2562 | Lev^{R} | | | | | | | | |
| | Cip^{R} | | | | | | | | |
| | MEM^{R} | | | | | | | | |
| | CAZ^{R} | | | | | | | | |
| P. aeruginosa | MDR | 1 | 2 | 1 | 64 | 32 | 16 | 32 | 8 |
| MMX 1381 | Lev^{R} | | | | | | | | |
| | Cip^{R} | | | | | | | | |
| | MEM^{R} | | | | | | | | |
| | CAZ^{R} | | | | | | | | |
| P. aeruginosa | MDR | 1 | 8 | 2 | 64 | 32 | 16 | 16 | 4 |
| MMX 3991 | Lev^{R} | | | | | | | | |
| | Cip | | | | | | | | |
| | MEM^{R} | | | | | | | | |
| | CAZ^{R} | | | | | | | | |
| A. baumannii | MDR; | 0.5 | 16 | 0.5 | 8 | 32 | 64 | >32 | 8 |
| MMX 4651 | OXA-27 | | | | | | | | |
| (NCTC 13304) | Lev^{R} | | | | | | | | |
| | Cip^{R} | | | | | | | | |
| | MEM^{R} | | | | | | | | |
| | CAZ^{R} | | | | | | | | |
| | Gm^{I} | | | | | | | | |
| A. baumannii | MDR | 0.5 | 16 | 0.5 | 64 | >64 | 64 | 32 | >64 |
| MMX 2592 | Ley^{R} | | | | | | | | |
| | Cip | | | | | | | | |
| | MEM^{R} | | | | | | | | |
| | CAZ^{R} | | | | | | | | |
| | Gm^{R} | | | | | | | | |
| A. baumannii | MDR | 1 | 16 | 0.5 | 32 | >64 | 64 | 32 | >64 |
| MMX 2593 | Ley^{R} | | | | | | | | |
| | Cip^{R} | | | | | | | | |
| | MEM^{R} | | | | | | | | |
| | CAZ^{R} | | | | | | | | |
| | Gm^{R} | | | | | | | | |
| A. baumannii | MDR | 1 | 16 | 1 | 1 | 4 | 4 | 16 | 0.5 |
| MMX 3372 | Cip^{R} | | | | | | | | |
| | MEM^{I} | | | | | | | | |
| | CAZ^{I} | | | | | | | | |
| A. baumannii | Sensitive | 1 | 16 | 0.5 | 0.12 | 0.5 | 0.5 | 4 | 0.12 |
| MMX 3373 | | | | | | | | | |

QC = quality control; VIM/IMP = metallo-beta lactamase type; OXA = type D extended-spectrum beta-lactamase; MDR = multi-drug resistant (based on resistance to at least 3 different classes of antibiotic); Lev^{R} = levofloxacin-resistant; CIP^{R} = Ciprofloxacin-resistant; MEM^{I} = meropenem intermediate resistance; MEM^{R} = meropenem-resistant; CAZ^{R} = ceftazidime-resistant; CAZ^{I} = ceftazidime intermediate resistance; Gm^{R} = gentamicin-resistant; Gm^{I} = gentamicin intermediate resistance.
¹CLSI QC ranges shown in parenthesis where applicable

TABLE 5
Minimal Inhibitory Concentration (MIC) Values for Microbion Bismuth Thiol
Test Agents and Comparators Against S. aureus and Enterococcus spp.

| | | MIC (μg/mL) | | | | | | | | | | | |
|---|---|---|---|---|---|---|---|---|---|---|---|---|---|
| | | MB- | MB- | MB- | Levo- | Cipro- | Genta- | Vanco- | | Ceftaz- | Clinda- | Eryth- | |
| Isolate | Type | 1-B3 | 2B | 6 | floxacin | floxacin | micin | mycin | Meropenem | idime | mycin | romycin | Oxacillin |
| S. aureus | QC; | ≤0.06 | 0.5 | ≤0.06 | 0.12 | 0.5 | 0.12 | 1 | 0.06 | 00 | 0.12 | 0.5 | 0.2 [symbol] |
| MMX 101 | MSSA | | | | (0.06- | (0.12- | (0.12- | (0.5- | (0.03- | (4- | (0.06- | (0.25- | (0.1 [symbol] |
| (ATCC 29213) | | | | | 0.5)¹ | 0.5) | 1) | 2) | 0.12) | 16) | 0.25) | 1) | 0.2 [symbol] |
| S. aureus | MRSA² | ≤0.06 | 0.25 | ≤0.06 | 4 | 16 | 0.12 | 2 | 4 | >32 | 0.12 | >64 | 64 |
| MMX 9203 | Lev^{R} | | | | | | | | | | | | |
| | Cip | | | | | | | | | | | | |
| | Erv^{R} | | | | | | | | | | | | |
| | Ox^{R} | | | | | | | | | | | | |
| S. aureus | MRSA | ≤0.06 | ≤0.06 | ≤0.06 | 4 | 16 | 0.25 | 1 | 4 | >32 | 0.12 | >64 | 64 |
| MMX 9204 | Lev^{R} | | | | | | | | | | | | |
| | Cip | | | | | | | | | | | | |
| | Erv^{R} | | | | | | | | | | | | |
| | Ox^{R} | | | | | | | | | | | | |
| S. aureus | MRSA | ≤0.06 | ≤0.06 | ≤0.06 | >64 | >64 | 0.25 | 2 | 64 | >32 | >32 | >64 | [symbol] |
| MMX 9205 | Lev^{R} | | | | | | | | | | | | |
| | Cip^{R} | | | | | | | | | | | | |
| | CC^{R} | | | | | | | | | | | | |
| | Ery^{R} | | | | | | | | | | | | |
| | Ox^{R} | | | | | | | | | | | | |
| S. aureus | MRSA | ≤0.06 | ≤0.06 | ≤0.06 | 32 | >64 | 0.25 | 2 | 32 | >32 | >32 | >64 | [symbol] |
| MMX 5373 | Ley^{R} | | | | | | | | | | | | |
| | Cip^{R} | | | | | | | | | | | | |
| | CC^{R} | | | | | | | | | | | | |
| | Ery^{R} | | | | | | | | | | | | |
| | Ox^{R} | | | | | | | | | | | | |
| S. aureus | MRSA | ≤0.06 | ≤0.06 | ≤0.06 | 32 | >64 | 0.25 | 0.25 | 8 | >32 | >32 | >64 | [symbol] |
| MMX 6311 | Lev^{R} | | | | | | | | | | | | |
| | Cip^{R} | | | | | | | | | | | | |
| | CC^{R} | | | | | | | | | | | | |
| | Ery^{R} | | | | | | | | | | | | |
| | Ox^{R} | | | | | | | | | | | | |
| E. faecalis | VRE | 0.5 | 2 | 0.5 | 32 | 64 | >64 | >64 | 8 | >32 | >32 | >64 | >64 |
| MMX 8960 | Lev^{R} | | | | | | | | | | | | |
| | Cip^{R} | | | | | | | | | | | | |
| | Ery^{R} | | | | | | | | | | | | |
| E. faecalis | VRE | 0.25 | 2 | 0.25 | 64 | 64 | 8 | >64 | 8 | >32 | >32 | >64 | 64 |
| MMX 8961 | Lev^{R} | | | | | | | | | | | | |
| | Cip^{R} | | | | | | | | | | | | |
| | Ery^{R} | | | | | | | | | | | | |
| E. faecalis | VRE | 0.5 | 2 | 0.25 | 64 | 64 | >64 | >64 | 2 | >32 | >32 | >64 | [symbol] |
| MMX 8962 | Lev^{R} | | | | | | | | | | | | |
| | Cip^{R} | | | | | | | | | | | | |
| | Ery^{R} | | | | | | | | | | | | |
| E. faecium | VRE | 2 | 4 | 1 | >64 | >64 | >64 | >64 | >64 | >32 | >32 | >64 | [symbol] |
| MMX 752 | vanA | | | | | | | | | | | | |
| | Lev^{R} | | | | | | | | | | | | |
| | Cip^{R} | | | | | | | | | | | | |
| | Ery^{R} | | | | | | | | | | | | |
| E. faecium | VRE | 1 | 2 | 0.5 | >64 | >64 | >64 | >64 | >64 | >32 | >32 | >64 | [symbol] |
| MMX 485 | vanA | | | | | | | | | | | | |
| | Lev^{R} | | | | | | | | | | | | |
| | Cip^{R} | | | | | | | | | | | | |
| | Ery^{R} | | | | | | | | | | | | |

QC = quality control; MSSA = methicillin-susceptible S. aureus; MRSA = methicillin-resistant S. aureus; VRE = vancomycin-resistant enterococci; vanA = vanA-type VRE (based on vancomycin- and teicoplanin-resistant phenotype); Lev^{R} = levofloxacin-resistant; CIP^{R} = Ciprofloxacin-resistant; CC^{R} = Clindamycin-resistant; Ery^{R} = Erythromycin-resistant; Ox^{R} = oxacillin-resistant
¹CLSI QC ranges shown in parenthesis where applicable
²MRSA do not have breakpoints for ceftazidime and meropenem - resistance is presumed. [symbol]
indicates data missing or illegible when filed

TABLE 6
Minimal Inhibitory Concentration (MIC) Values for Microbion Bismuth Thiol Test Agents and Comparators Against Streptococcus spp.

| | | MIC (μg/mL) | | | | | | | | | | |
|---|---|---|---|---|---|---|---|---|---|---|---|---|
| | | MB- | MB- | | Levo- | Cipro- | | Vanco- | Trimeth/ | Clin- | Eryth- | Peni- |
| Isolate | Type | 1-B3 | 2B | MB-6 | floxacin | floxacin | Meropenem | mycin | Sulfa | damycin | romycin | cillin |
| S. pneumoniae | QC | 0.5 | 8 | 0.5 | 0.5 | 0.5 | 0.06 | 0.25 | 0.25/4.8 | 0.06 | 0.03 | 0.25 |
| MMX 6837 | | | | | (0.5-2)¹ | | (0.06-0.25) | (0.12-0.5) | (0.12/2.4-1/19) | (0.03-0.12) | (0.03-0.12) | (0.25-1) |
| (ATCC 49619) | | | | | | | | | | | | |
| S. pneumoniae | Lev^{R} | 1 | 8 | 0.5 | 8 | 32 | 0.5 | 0.5 | 4/76 | >8 | >8 | 2 |
| MMX 880 | SXT^{R} | | | | | | | | | | | |
| | CC^{R} | | | | | | | | | | | |
| | Ery^{R} Pen^{R} | | | | | | | | | | | |
| S. pneumoniae | Mem^{R} | 1 | 1 | 8 | 1 | 1 | 1 | 0.5 | 16/304 | >8 | >8 | 4 |
| MMX 937 | SXT^{R} | | | | | | | | | | | |
| | CC^{R} | | | | | | | | | | | |
| | Ery^{R} Pen^{R} | | | | | | | | | | | |
| S. pneumoniae | CC^{R} | 1 | 8 | 1 | 1 | 4 | 0.03 | 0.5 | 2/38 | >8 | >8 | 0.12 |
| MMX 3959 | Ery^{R} | | | | | | | | | | | |
| S. pneumoniae | Mem^{R} | 1 | 8 | 0.5 | 1 | 2 | 1 | 0.25 | 16/304 | >8 | >8 | 4 |
| MMX 5440 | SXT^{R} | | | | | | | | | | | |
| | CC^{R} | | | | | | | | | | | |
| | Ery^{R} Pen^{R} | | | | | | | | | | | |
| S. pneumoniae | Mem^{R} | 0.5 | 8 | 0.5 | 1 | 2 | 1 | 0.25 | 8/152 | >8 | >8 | 4 |
| MMX 5445 | SXT^{R} | | | | | | | | | | | |
| | CC^{R} | | | | | | | | | | | |
| | Ery^{R} Pen^{R} | | | | | | | | | | | |
| S. pneumoniae | Mem^{R} | 0.25 | 8 | 0.5 | 1 | 1 | 1 | 0.25 | 8/152 | >8 | >8 | 4 |
| MMX 8133 | SXT^{R} | | | | | | | | | | | |
| | CC^{R} | | | | | | | | | | | |
| | Ery^{R} Pen^{R} | | | | | | | | | | | |
| S. pyogenes | Ery^{R} | 0.12 | 2 | 0.12 | 0.5 | 0.5 | ≤0.008 | 0.5 | 0.12/2.4 | 0.06 | >8 | ≤0.008 |
| MMX 3068 | | | | | | | | | | | | |
| S. pyogenes | E^{R}Y^{R} | 0,25 | 1 | 0.5 | 0.25 | 0.12 | ≤0.008 | 0.5 | 0.12/2.4 | 1 | >8 | ≤0.008 |
| MMX 3820 | CLI^{R} | | | | | | | | | | | |
| S. pyogenes | E^{R}Y^{R} | 0.03 | 0.25 | 0.03 | 0.12 | 0.25 | ≤0.008 | 0.5 | 0.06/1.2 | 0.12 | >8 | ≤0.008 |
| MMX 3919 | | | | | | | | | | | | |
| S. pyogenes | E^{R}Y^{R} | 0.25 | 1 | 0.5 | 0.5 | 0.5 | ≤0.008 | 0.5 | 0.25/4.8 | >8 | >8 | ≤0.008 |
| MMX 3929 | CC^{R} | | | | | | | | | | | |
| S. pyogenes | E^{R}Y^{R} | 0.5 | 1 | 1 | 0.25 | 0.25 | ≤0.008 | 0.5 | 0.06/1.2 | >8 | >8 | ≤0.008 |
| MMX 5091 | CC^{R} | | | | | | | | | | | |
| S. agalactiae | E^{R}Y^{R} | 0.5 | 4 | 0.25 | 0.5 | 0.5 | 0.03 | 0.5 | 0.12/2.4 | >8 | 8 | 0.03 |
| MMX 3741 | CC^{R} | | | | | | | | | | | |
| S. agalactiae | E^{R}Y^{R} | 0.25 | 4 | 0.25 | 1 | 1 | 0.06 | 0.5 | 0.12/2.4 | >8 | >8 | 0.06 |
| MMX 3743 | CC^{R} | | | | | | | | | | | |
| S. agalactiae | E^{R}Y^{R} | 0.5 | 4 | 0.25 | 0.5 | 1 | 0.06 | 0.5 | 0.12/2.4 | >8 | >8 | 0.06 |
| MMX 4077 | CC^{R} | | | | | | | | | | | |
| S. agalactiae | E^{R}Y^{R} | 1 | 8 | 0.5 | 1 | 1 | 8 | 2 | 0.12/2.4 | >8 | >8 | 2 |
| MMX 4079 | CC^{R} Pen^{R} | | | | | | | | | | | |
| | MEM^{R} | | | | | | | | | | | |
| S. agalactiae | E^{R}Y^{R} | 0.25 | N | 0.25 | 0.5 | 0.5 | 0.06 | 1 | 0.12/2.4 | >8 | >8 | 0.06 |
| MMX 4086 | CC^{R} | | | | | | | | | | | |

QC = quality control; Trimeth = trimethoprim; Sulfa = sulfamethoxazole; MDR = multi-drug resistant (based on resistance to at least 3 different classes of antibiotic); Ery^{R} = erythromycin-resistant; CC^{R} = clindamycin-resistant; SXT^{R} = Trimethoprim/Sulfamethoxazole-resistant; MEM^{R} = Meropenem-resistant; Pen^{R} = Penicillin-resistant
¹CLSI QC ranges shown in parenthesis where applicable

TABLE 7
Minimal Inhibitory Concentration (MIC) Values for Microbion Bismuth
Thiol Test Agents and Comparators Against N. gonorrhoeae

| | | MIC (μg/mL) | | | | |
|---|---|---|---|---|---|---|
| Isolate | Type | MB-1-B3 | MB-2B | MB-6 | Ciprofloxacin | Ceftriaxone |
| N. gonorrhoeae | QC | 0.06 | 0.12 | 0.06 | 0.008 | 0.015 |
| MMX 683 | | | | | (0.001-0.008)¹ | (0.004-0.015) |
| (ATCC 49226) | | | | | | |
| N. gonorrhoeae | CIPR | 0.12 | 0.12 | 0.12 | >8 | 0.06 |
| MMX 6791 | | | | | | |
| N. gonorrhoeae | CIPR | 0.12 | 0.12 | 0.12 | >8 | 0.06 |
| MMX 6792 | | | | | | |
| N. gonorrhoeae | CIPR | 0.12 | 0.5 | 0.25 | >8 | 0.03 |
| MMX 6793 | | | | | | |
| N. gonorrhoeae | CTX NS | 0.06 | 0.5 | 0.06 | 0.03 | 1 |
| MMX 6757 | | | | | | |

QC = quality control; Cip^{R} = ciprofloxacin-resistant; CTX NS = ceftriaxone non-susceptible
¹CLSI QC ranges shown in parenthesis where applicable

TABLE 8
Minimal Inhibitory Concentration (MIC) Values for Microbion
Bismuth Thiol Test Agents and Comparators Against Anaerobes

| | | MIC (μg/mL) | | | | | |
|---|---|---|---|---|---|---|---|
| Isolate | Type | MB-1-B3 | MB-2B | MB-6 | Clindamycin | Metronidazole | Fidaxomicin |
| B. fragilis | QC | 2 | 8 | 1 | 1 | 0.5 | >64 |
| MMX 123 | | | | | (0.5-2)¹ | (0.25-1) | |
| (ATCC 25285) | | | | | | | |
| C. difficile | QC | 4 | 8 | 4 | 4 | 0.5 | 0.25 |
| MMX 4381 | CC^{I} | | | | (2-8) | (0.12-0.5) | (0.06-0.25) |
| (ATCC 700057) | | | | | | | |
| C. difficile | ribotype 012 | 4 | 8 | 2 | >64 | 0.5 | 0.5 |
| MMX 5681 | CC^{R} | | | | | | |
| (NCTC 13307) | | | | | | | |
| C. difficile | ribotype 027 | 1 | 2 | 2 | 4 | >64 | 4 |
| MMX 5680 | CC^{I} | | | | | | |
| (NCTC 13336) | METR | | | | | | |
| C. difficile | ribotype 255 | 4 | 8 | 2 | 4 | 0.5 | 0.5 |
| MMX 8272 | CC^{I} | | | | | | |
| C. difficile | ribotype 005 | 4 | 16 | 2 | >64 | 0.5 | 0.5 |
| MMX 8279 | CC^{R} | | | | | | |
| C. difficile | ribotype 010 | 2 | 8 | 2 | >64 | 8 | 0.5 |
| MMX 8281 | CC^{R} | | | | | | |

QC = quality control; CC^{I} = Clindamycin intermediate resistance; CC^{R} = Clindamycin-resistant; MET^{R} = Metronidazole-resistant
¹CLSI QC ranges shown in parenthesis where applicable

TABLE 9
Minimal Inhibitory Concentration (MIC) Values for Microbion Bismuth
Thiol Test Agents and Comparators Against Candida species

| | | MIC¹ (μg/mL) | | | | |
|---|---|---|---|---|---|---|
| Isolate | Type | MB-1-B3 | MB-2B | MB-6 | Fluconazole | Amphotericin B |
| C. parapsilosis | QC | 0.5, 1 | 0.5, 1 | 0.5, 1 | 2, 2 | 0.5, 1 |
| MMX 2323 | | | | | (0.5-4, 1-4)² | (0.25-2, 0.5-4) |
| (ATCC 22019) | | | | | | |
| C. parapsilosis | FLU^{R} | 0.5, 0.5 | 0.5, 0.5 | 0.25, 0.5 | 32, 32 | 0.5, 1 |
| MMX 7370 | | | | | | |
| C. parapsilosis | FLU^{R} | 0.5, 1 | 0.25, 1 | 0.25, 0.5 | 32, 64 | 0.5, 0.5 |
| MMX 7555 | | | | | | |
| C. albicans | Sensitive | 4, 16 | 2, 2 | 2, 2 | 0.5, 0.5 | 0.5, 0.5 |
| MMX 7039 | | | | | | |
| C. albicans | Sensitive | 2, 16 | 1, 4 | 2, 2 | 0.25, 0.5 | 0.25, 0.5 |
| MMX 7055 | | | | | | |
| C. glabrata | FLU^{R} | 0.5, 1 | 0.5, 1 | 0.5, 1 | 32, 64 | 0.5, 1 |
| MMX 7086 | | | | | | |
| C. glabrata | FLU^{R} | 0.5, 1 | 0.25, 1 | 0.25, 0.5 | >64, >64 | 0.5, 1 |
| MMX 7318 | | | | | | |
| C. tropicalis | FLU^{R} | 4, 16 | 2, 4 | 4, 4 | 64, >64 | 0.5, 1 |
| MMX 7247 | | | | | | |
| C. tropicalis | FLU^{R} | 16, 32 | 4, 4 | 4, 8 | 32, >64 | 0.5, 1 |
| MMX 7248 | | | | | | |
| C. tropicalis | FLU^{R} | 2, 16 | 1, 2 | 2, 2 | 64, >64 | 0.5, 0.5 |
| MMX 7360 | | | | | | |

QC = quality control; FLU^{R} = fluconazole-resistant
¹MIC reported after incubation at 24 and 48 hr
²CLSI QC ranges shown in parenthesis where applicable

### REFERENCES

1.) Centers for Disease Control and Prevention. Antibiotic resistance threats in the United States, 2013. Available from http://www.cdc.gov/drugresistance/pdf/ar-threats-2013-508.pdf. Accessed on Jun. 13, 2016.
2.) Boucher H W, Talbot G H, Bradley J S, Edwards J E, Gilbert D, Rice L B, Scheld M, Spellberg B, Bartlett J. Bad bugs, no drugs: no ESKAPE! An update from the Infectious Diseases Society of America. Clin Infect Dis 2009; 48: 1-12.
3.) Rice L B. Federal funding for the study of antimicrobial resistance in nosocomial pathogens: no ESKAPE. J Infect Dis 2008; 197: 1079-1081.
4.) Clinical and Laboratory Standards Institute (CLSI). Methods for Dilution Antimicrobial Susceptibility Tests for Bacteria That Grow Aerobically; Approved Standard—Tenth Edition. Clinical and Laboratory Standards Institute document M07-A10. CLSI, 940 West Valley Road, Suite 1400, Wayne, Pennsylvania 19087-1898 USA, 2015.
5.) CLSI. Performance Standards for Antimicrobial Susceptibility Testing; Twenty-Sixth Informational Supplement. CLSI document M100-S26. CLSI, 940 West Valley Road, Suite 1400, Wayne, Pennsylvania 19087 USA, 2016.
6.) CLSI. Methods for Antimicrobial Susceptibility Testing of Anaerobic Bacteria; Approved Standard-Eighth Edition. CLSI document M11-A8. CLSI, 940 West Valley Road, Suite 1400, Wayne, Pennsylvania 19087-1898 USA, 2012.
7.) CLSI. Reference Method for Broth Dilution Antifungal Susceptibility Testing of Yeasts; Approved Standard-Third Edition. CLSI document M27-A3. CLSI, 940 West Valley Road, Suite 1400, Wayne, Pennsylvania 19087-1898 USA, 2008.
8.) CLSI. Reference Method for Broth Dilution Antifungal Susceptibility Testing of Yeasts; Fourth Informational Supplement CLSI document M27-S4. CLSI, 940 West Valley Road, Suite 1400, Wayne, Pennsylvania 19087-1898 USA, 2012.

### Example 6: In vitro Activity of Bismuth Thiols Against Vancomycin-Intermediate S. aureus and β-lactamase Producing Gram-negative Bacteria

### Introduction

The in vitro activity of BisEDT and two additional bismuth-thiol investigational agents (MB-2B and MB-6) was determined for isolates of Escherichia coli, Klebsiella pneumoniae, Pseudomonas aeruginosa and Acinetobacter baumannii characterized for extended-spectrum β-lactamases (ESBL) and/or carbapenem resistance. In addition, vancomycin-intermediate Staphylococcus aureus (VISA) were evaluated. The majority of the isolates tested in the current study were multidrug-resistant (MDR) as defined by resistance to at least three different antibiotic classes. Susceptibility to the investigational compounds and relevant comparators was determined by broth microdilution conducted in accordance with guidelines from the Clinical and Laboratory Standards Institute (CLSI; 2,3).

### Materials and Methods

Test Compounds: The test agents BisEDT (MB-1-B3; Lot No. ED268-1-11-01), MB-2B, and MB-6 were stored at room temperature, in the dark, until assayed. The solvent and diluent for the test agents was DMSO (Sigma; St. Louis, MO; Lot No. SHBB9319V) and the prepared stock concentration was 6,464 μg/mL (101× the final test concentration).

Comparator drugs were supplied and shown in Table 10 below:

TABLE 10
Comparator drugs

| | | Catalog | | Solvent/ |
|---|---|---|---|---|
| Test Agents | Supplier | Number | Lot Number | Diluent |
| BisEDT | Microbion | — | ED268-1-11-01 | DMSO/DMSO |
| MB-2B | Microbion | — | TA-8-167-01 | DMSO/DMSO |
| MB-6 | Microbion | — | 5-21-14 | DMSO/DMSO |
| Amikacin | Sigma | A2324-5G | 058K0803 | H₂O/H₂O |
| Ceftazidime | Sigma | C3809-1G | 076M4770V | H₂O/H₂O |
| Clavulanate | Sigma | 33454- | STBH5214 | Phos. buff. |
| | | 100MG | | pH 6.0 |
| Clindamycin | Sigma | C5269- | 021M1533 | H₂O/H₂O |
| | | 100MG | | |
| Daptomycin | Cubist | — | MCB2009 | H₂O/H₂O |
| Levofloxacin | Sigma | 28266-1G-F | BCBF7004V | H₂O + |
| | | | | NaOH/H₂O |
| Linezolid | Selleck | S1408 | S140802 | H₂O/H₂O |
| | Chemicals | | | |
| Meropenem | USP | 1392454 | J0K434 | H₂O/H₂O |
| Vancomycin | Sigma | V2002-1G | 080M1341V | H₂O/H₂O |

Test compounds were evaluated at a concentration range of 0.06-64 μg/mL. For Gram-negative test isolates, amikacin and ceftazidime (alone and with clavulanate at a fixed concentration of 4 μg/mL) were evaluated over a concentration range of 0.06-64 μg/mL; meropenem and levofloxacin were evaluated over a concentration range of 0.008-8 μg/mL. For the testing of S. aureus, clindamycin, daptomycin, levofloxacin and vancomycin were evaluated at a concentration range of 0.008-8 μg/mL; linezolid was tested from 0.03-32 μg/mL.

Organisms: The test organisms as shown in Tables 11-15 consisted of clinical isolates from the Micromyx (MMX) repository and reference strains from the American Type Culture Collection (ATCC; Manassas, VA), National Collection of Type Cultures (NCTC; Public Health England, Salisbury, UK), the Network on Antimicrobial Resistance in Staphylococcus aureus (NARSA; BEI Resources, Manassas, VA), and the Centers for Disease Control and Prevention (CDC; Atlanta, GA). The test organisms were maintained frozen at −80° C. Prior to testing, the isolates were cultured on Tryptic Soy Agar with 5% sheep blood (BAP; Becton Dickson [BD]/BBL; Sparks, MD; Lot Nos. 9080650 and 9108563) at 35° C. Relevant ATCC quality control (QC) organisms (Table 16) were included during testing in accordance with CLSI guidelines (3). Further details on the genetic characterization of the isolates where available can be found in Table 17.

Media: Cation-adjusted Mueller Hinton broth (CAMHB; BD; Lot No. 8190586) was used as the medium for testing (2, 3). For testing daptomycin, calcium was supplemented with 25 mg/mL Ca²⁺, resulting in a final concentration of 50 mg/mL Ca²⁺ (2, 3).

MIC Assay Procedure: MIC values were determined using a broth microdilution procedure described by CLSI (2, 3). Automated liquid handlers (Multidrop 384, Labsystems, Helsinki, Finland; Biomek 2000 and Biomek F X, Beckman Coulter, Fullerton CA) were used to conduct serial dilutions and liquid transfers.

To prepare the drug mother plates, which would provide the serial drug dilutions for the replicate daughter plates, the wells of columns 2 through 12 of standard 96-well microdilution plates (Costar 3795) were filled with 150 μl of the appropriate diluent for each row of drug. The test articles and comparator compounds (300 μl at 101× the highest concentration to be tested) were dispensed into the appropriate wells in column 1. The Biomek 2000 was then used to make 2-fold serial dilutions in the mother plates from column 1 through column 11. The wells of column 12 contained no drug and served as the organism growth control wells for the assay.

The daughter plates were loaded with 190 μL per well of RPMI using the Multidrop 384. The test panels were prepared on the Biomek FX instrument which transferred 2 μL of drug solution from each well of a mother plate to the corresponding well of each daughter plate in a single step.

A standardized inoculum of each test organism was prepared per CLSI methods to equal a 0.5 McFarland standard, followed by a dilution of 1:20. The plates were then inoculated with 10 μL of the diluted inoculum using the Biomek 2000 from low to high drug concentration resulting in a final concentration of approximately 5×10⁵ CFU/mL.

Plates were stacked 3-4 high, covered with a lid on the top plate, placed into plastic bags, and incubated at 35° C. for 16 to 20 hr (vancomycin was read for S. aureus after 24 hr incubation time). The MIC was recorded as the lowest concentration of drug that inhibited visible growth of the organism.

### Results and Discussion

As shown in Table 16, results for BisEDT and comparators were within CLSI established QC ranges against the relevant ATCC QC isolates, thus validating the susceptibility testing conducted during the study.

The activity of BisEDT, MB-2B, and MB-6, against the resistant Gram-negative bacilli are shown by species in Tables 11-14. BisEDT maintained potent activity with MIC values of 0.5-2 μg/mL across isolates with the exception of one isolate of P. aeruginosa (CDC 241) which had an MIC of 4 μg/mL (Table 13) and several isolates of K. pneumoniae with MIC values of 4-8 μg/mL (Table 12). The activity of BisEDT was not impacted by β-lactamase production or resistance to aminoglycosides (amikacin MIC≥64 μg/mL), fluoroquinolones (levofloxacin MIC≥2, 4, and 8≥64 for Enterobacteriaceae, P. aeruginosa, and A. baumannii, respectively).

Overall, MB-6 had MIC values that were either identical or within 2-fold of those observed with BisEDT; exceptions included select K. pneumoniae where MB-6 MIC values were lower than those of BisEDT. The activity of BisEDT and MB-6 was greater than that of MB-2B, particularly for P. aeruginosa and A. baumannii. The MIC values observed with BisEDT, MB-2B, and MB-6 against Gram-negative bacilli were comparable to those observed in prior studies (1, 4).

The activity of BisEDT, MB-2B, and MB-6 against VISA is shown in Table 15. BisEDT had potent MIC values of ≤0.06-0.25 μg/mL against these isolates. As with Gram-negative bacilli, the activity of BisEDT was comparable to that observed with MB-6 and was greater than that observed with MB-2B. Of note, two of the VISA isolates (NRS 13 and 27) from NARSA had vancomycin MIC values of 2 μg/mL, which indicated that during testing in this study they tested as vancomycin-susceptible. The other two isolates with vancomycin MIC values in the susceptible range (NRS 2 and 24) are heterogenous VISA (hVISA) for which vancomycin MIC values are known to vary. Resistance to levofloxacin and clindamycin (MIC values≥4 μg/mL) was observed with all isolates except NRS 13 and did not impact BisEDT activity. Two of the isolates were also non-susceptible to daptomycin (NRS 13 and 22); all were susceptible to linezolid (MIC values≤4 μg/mL). The activity observed with BisEDT in this study was comparable to that observed previously (1, 4).

In summary, BisEDT showed potent activity against genetically characterized β-lactam-resistant Gram-negative bacilli, the majority of which were MDR, and reference isolates of VISA. The activity of BisEDT was not impacted by resistance to β-lactams or any other class evaluated in this study. Finally, the activity of BisEDT and MB-6 was comparable against the evaluated bacteria and exceeded that observed with MB-2B.

TABLE 11
Activity of BisEDT, MB-2B, MB-6 and comparators against Escherichia coli

| | | MIC (μg/mL) | | | | | | | |
|---|---|---|---|---|---|---|---|---|---|
| | | | | | | CAZ/ | | | |
| Isolate | β-lactamase Type | BisEDT | MB-2B | MB-6 | CAZ | CLAV | MEM | LVX | AMK |
| ATCC 35218 | ESBL | 1 | 2 | 1 | 0.12 | ≤0.06/4 | ≤0.008 | ≤0.008 | 1 |
| MMX 5755 | ESBL | 1 | 4 | 2 | >64 | 1/4 | 0.015 | 8 | 16 |
| MMX 5756 | ESBL | 1 | 2 | 1 | >64 | 0.5/4 | ≤0.008 | 4 | 64 |
| MMX 5758 | ESBL | 1 | 4 | 1 | 16 | 0.25/4 | ≤0.008 | 8 | 1 |
| CDC 451 | KPC | 1 | 2 | 1 | 64 | 16/4 | 1 | >8 | 4 |
| MMX 5745 | KPC | 1 | 4 | 2 | 32 | 16/4 | 2 | 8 | 0.5 |
| CDC 114 | ESBL/KPC | 2 | 2 | 2 | >64 | 64/4 | 2 | 4 | 1 |
| ATCC BAA-2471 | NDM | 1 | 2 | 2 | >64 | >64/4 | >8 | >8 | 64 |
| CDC 435 | NDM | 1 | 2 | 1 | >64 | >64/4 | >8 | >8 | >64 |
| CDC 503 | ESBL/NDM | 1 | 2 | 2 | >64 | >64/4 | 8 | >8 | >64 |
| CDC 118 | ESBL/NDM | 1 | 4 | 1 | >64 | >64/4 | 0.06 | 8 | >64 |

ATCC = American Type Culture Collection, MMX = Micromyx, CDC = Centers for Disease Control and Prevention, ESBL = extended-spectrum β-lactamase, KPC = K. pneumoniae carbapenemase, NDM = New Delhi metallo-β-lactamase, CAZ = ceftazidime, CLAV = clavulanate, MEM = meropenem, LVX = levofloxacin, AMK = amikacin

TABLE 12
Activity of BisEDT, MB-2B, MB-6 and comparators against Klebsiella pneumoniae

| | | MIC (μg/mL) | | | | | | | |
|---|---|---|---|---|---|---|---|---|---|
| | | | | | | CAZ/ | | | |
| Isolate | β-lactamase Type | BisEDT | MB-2B | MB-6 | CAZ | CLAV | MEM | LVX | AMK |
| MMX 9029 | ESBL | 4 | 2 | 2 | 4 | 0.12/4 | 0.015 | 0.12 | 0.5 |
| CDC 112 | KPC | 2 | 4 | 2 | >64 | >64/4 | >8 | 4 | 16 |
| ATCC BAA-1705 | ESBL/KPC | 2 | 4 | 1 | >64 | >64/4 | 8 | >8 | 16 |
| CDC 113 | ESBL/KPC | 8 | 32 | 1 | >64 | >64/4 | >8 | 4 | 16 |
| CDC 115 | ESBL/KPC | 8 | 16 | 2 | >64 | >64/4 | >8 | >8 | 0.5 |
| CDC 120 | ESBL/KPC | 2 | 4 | 2 | >64 | 64/4 | >8 | >8 | 16 |
| CDC 126 | ESBL/KPC | 8 | 32 | 2 | 4 | 8 | 4 | 0.015 | 2 |
| CDC 129 | ESBL/KPC | 2 | 4 | 4 | >64 | >64/4 | 8 | >8 | 32 |
| CDC 135 | ESBL/VIM | 4 | 8 | 4 | >64 | >64/4 | 1 | >8 | 16 |
| CDC 138 | ESBL/NDM | 4 | 16 | 2 | >64 | >64/4 | >8 | >8 | >64 |
| CDC 158 | ESBL/NDM/OXA | 2 | 4 | 2 | >64 | >64/4 | >8 | 4 | 2 |

ATCC = American Type Culture Collection, MMX = Micromyx, CDC = Centers for Disease Control and Prevention, ESBL = extended-spectrum β-lactamase, KPC = K. pneumoniae carbapenemase, NDM = New Delhi metallo-β-lactamase, VIM = metallo-β-lactamase, OXA = class D carbapenemases, CAZ = ceftazidime, CLAV = clavulanate, MEM = meropenem, LVX = levofloxacin, AMK = amikacin

TABLE 13
Activity of BisEDT, MB-2B, MB-6 and comparators
against Pseudomonas aeruginosa

| | | MIC (μg/mL) | | | | | | | |
|---|---|---|---|---|---|---|---|---|---|
| | B-lactamase | | | | | CAZ/ | | | |
| Isolate | Type | BisEDT | MB-2B | MB-6 | CAZ | CLAV | MEM | LVX | AMK |
| CDC 356 | KPC | 1 | 4 | 1 | 64 | 64/4 | >8 | 0.12 | 2 |
| CDC 439 | IMP | 2 | 4 | 1 | >64 | >64/4 | >8 | 8 | >64 |
| CDC 444 | VIM | 1 | 4 | 1 | 32 | 32/4 | >8 | 8 | >64 |
| CDC 457 | VIM | 1 | 32 | 2 | >64 | 64/4 | >8 | >8 | 2 |
| CDC 231 | KPC/OXA | 2 | 16 | 2 | >64 | >64/4 | >8 | >8 | 8 |
| CDC 230 | VIM/OXA | 0.5 | 4 | 2 | 64 | 32/4 | >8 | 8 | >64 |
| CDC 241 | IMP/OXA | 4 | 32 | 8 | >64 | >64/4 | >8 | 8 | 32 |
| CDC 246 | NDM/OXA | 2 | 8 | 2 | >64 | >64/4 | >8 | >8 | >64 |
| CDC 250 | NDM/OXA | 2 | 8 | 2 | >64 | >64/4 | >8 | >8 | >64 |
| CDC 516 | KPC/AmpC | 1 | 4 | 1 | 64 | 64/4 | >8 | 0.25 | 2 |
| CDC 518 | KPC/AmpC | 1 | 4 | 1 | 32 | 32/4 | >8 | >8 | 16 |

CDC = Centers for Disease Control and Prevention, KPC = K. pneumoniae carbapenemase, NDM = New Delhi metallo-β-lactamase, IMP = metallo-β-lactamase, VIM = metallo-β-lactamase, OXA = class D carbapenemases, AmpC = class C cephalosporinase, CAZ = ceftazidime, CLAV = clavulanate, MEM = meropenem, LVX = levofloxacin, AMK = amikacin

TABLE 14
Activity of BisEDT, MB-2B, MB-6 and comparators against Acinetobacter baumannii

| | | MIC (μg/mL) | | | | | | | |
|---|---|---|---|---|---|---|---|---|---|
| | β-lactamase | | | | | CAZ/ | | | |
| Isolate | Type | BisEDT | MB-2B | MB-6 | CAZ | CLAV | MEM | LVX | AMK |
| NCTC 13304 | OXA | 0.5 | >16 | 0.5 | >64 | >64/4 | >8 | 1 | 0.5 |
| CDC 307 | OXA | 1 | 32 | 0.5 | 64 | 64/4 | 8 | 8 | >64 |
| CDC 311 | OXA | 1 | 32 | 0.5 | >64 | >64/4 | >8 | 2 | >64 |
| CDC 312 | OXA | 1 | 32 | 0.5 | >64 | >64/4 | 4 | 2 | 1 |
| CDC 273 | ESBL/OXA | 1 | 32 | 0.5 | 64 | 64/4 | >8 | >8 | >64 |
| CDC 274 | ESBL/OXA | 1 | 32 | 0.5 | >64 | >64/4 | >8 | 8 | 16 |
| CDC 275 | ESBL/OXA | 0.5 | 32 | 0.5 | >64 | >64/4 | >8 | 4 | >64 |
| CDC 277 | ESBL/OXA | 1 | >16 | 1 | >64 | >64/4 | >8 | 8 | 16 |
| CDC 284 | ESBL/OXA | 1 | >16 | 1 | 64 | 16/4 | >8 | 8 | 32 |
| CDC 308 | ESBL/OXA | 1 | 32 | 0.5 | 64 | 64/4 | 8 | 4 | >64 |
| CDC 313 | ESBL/OXA | 1 | 32 | 0.5 | >64 | >64/4 | >8 | 2 | 4 |

NCTC = National Collection of Type Cultures, CDC = Centers for Disease Control and Prevention, ESBL = extended-spectrum β-lactamase, OXA = class D carbapenemases, CAZ = ceftazidime, CLAV = clavulanate, MEM = meropenem, LVX = levofloxacin, AMK = amikacin

TABLE 15
Activity of BisEDT, MB-2B, MB-6 and comparators against
vancomycin-intermediate Staphylococcus aureus

| | MIC (μg/mL) | | | | | | | |
|---|---|---|---|---|---|---|---|---|
| Isolate | BisEDT | MB-2B | MB-6 | VAN | DAP | CLI | LVX | LZD |
| NRS 1 (hVISA) | 0.12 | 1 | 0.12 | 4 | 1 | >8 | 4 | 1 |
| NRS 2 | ≤0.06 | 0.5 | ≤0.06 | 0.5 | 0.25 | >8 | 4 | 4 |
| NRS 3 | 0.25 | 2 | 0.25 | 8 | 1 | >8 | >8 | 1 |
| NRS 22 | 0.25 | 2 | 0.25 | 4 | 2 | >8 | 8 | 1 |
| NRS 4 | 0.25 | 1 | 0.25 | 4 | 0.5 | >8 | 4 | 1 |
| NRS 13 | 0.12 | 1 | 0.12 | 2 | 2 | 0.06 | 0.12 | 2 |
| NRS 18 | 0.25 | 1 | 0.25 | 4 | 0.5 | >8 | 4 | 1 |
| NRS 24 (hVISA) | 0.25 | 1 | 1 | 2 | 0.5 | >8 | >8 | 2 |
| NRS 27 | 0.25 | 1 | 0.25 | 2 | 0.25 | >8 | 8 | 2 |

NRS = Network on Antimicrobial Resistance in Staphylococcus aureus, MMX = Micromyx, VISA = vancomycin-intermediate S. aureus, hVISA = heterogenous vancomycin-intermediate S. Aureus, VAN = vancomycin, DAP = daptomycin, CLI = clindamycin, LVX = levofloxacin, LZD = linezolid

TABLE 16
Activity of Bis-EDT, MB-2B, MB-6 and comparators against relevant ATCC QC organisms

| | | MIC (μg/mL) | | | | | | | |
|---|---|---|---|---|---|---|---|---|---|
| | | | | | | CAZ + | | | |
| Organism | Isolate | BisEDT | MB-2B | MB-6 | CAZ | CLAV | MEM | LVX | AMK |
| E. coli | ATCC 25922 | 0.5 | 2 | 1 | 0.25 | 0.25/4 | ≤0.008 | ≤0.008 | 1 |
| | | (0.5-4)1 | | | (0.06-0.5) | | (0.008-0.06) | (0.008-0.06) | |
| | ATCC 35218 | 1 | 2 | 1 | 0.12 | ≤0.06/4 | ≤0.008 | ≤0.008 | 1 |
| | | | | | | | (0.008-0.06) | | |
| K. pneumoniae | ATCC BAA-1705 | 2 | 4 | 1 | >64 | >64/4 | 8 | >8 | 16 |
| | | | | | | | (8-64) | | |
| P. aeruginosa | ATCC 27853 | 1 | 2 | 1 | 2 | 2/4 | 0.25 | 0.5 | 2 |
| | | (0.5-4) | | | (1-4) | | (0.12-1) | (0.5-4) | (1-4) |
| | | MIC (μg/mL) | | | | | | | |
| Organism | Isolate | BisEDT | MB-2B | MB-6 | VAN | DAP | CLI | LVX | LZD |
| S. aureus | ATCC 29213 | 0.12 | 1 | 0.12 | 0.5 | 0.5 | 0.25 | 0.06 | 4 |
| | | (0.12-1) | | | (0.5-2) | (0.12- | (0.06-0.25) | (0.06-0.5) | (1-4) |

ATCC = American Type Culture Collection, CAZ = ceftazidime, CLAV = clavulanate, MEM = meropenem, LVX = levofloxacin, AMK = amikacin, VAN = vancomycin, DAP = daptomycin, CLI = clindamycin, LVX = levofloxacin, LZD = linezolid QC ranges in parentheses

TABLE 17
Available genetic characterization data on test isolates

| Organism | Isolate | Genetic Characterization Information |
|---|---|---|
| E. coli | ATCC 35218 | TEM-1 |
| | BAA-2471 | NDM-1 |
| | CDC 435 | NDM |
| | CDC 451 | KPC |
| | MMX 5745 | KPC, TEM, DFR |
| | MMX 5755 | SHV, TEM, OXA-1, AAD, ANT, SUL1, SUL2, GYR |
| | MMX 5756 | SHV, TEM, OXA-9, AAD, SUL2, GYR |
| | MMX 5758 | SHV, TEM, CTX-M-1, GYR |
| | CDC 503 | CTX-M-15, NDM-1, OXA-181 |
| | CDC 114 | aadB, cmlA1, dfrA5, KPC-3, strA, strB, sul1, sul2, TEM-1B |
| | CDC 118 | aac(3)-IIa, catA1, CMY-6, dfrA29, NDM-1, OmpF, OXA-2, rmtC, strA, strB, sul1, TEM-1A |
| K. pneumoniae | BAA-1705 | KPC-2, TEM, SHV |
| | MMX 9029 | CTX-M1, SHV, TEM, AAC, SUL2 |
| | CDC 112 | aac(6′), aph(3′), aph(4), catA1, cmlA1, dfrA12, KPC-3, mph(A), oqxA, oqxA, oqxB, sul1, sul3 |
| | CDC 113 | aac(6′)-Ib, aph(3′)-Ia, aph(4)-Ia, catA1, cmlA1, dfrA12, KPC-3, mph(A), OmpK35, OmpK36, oqxA, oqxA, |
| | | oqxB, SHV-11, sul1, sul3 |
| | CDC 115 | aph(3′)-Ia, aph(4)-Ia, catA1, cmlA1, dfrA12, KPC-3, mph(A), OmpK35, oqxA, oqxA, oqxB, sul1, sul3, TEM-1A |
| | CDC 120 | aac(6′)-33, aac(6′)-Ib, aadA2, aadB, aph(3′)-Ia, dfrA12, KPC-2, mph(A), OmpK35, oqxA, oqxA, oqxB, |
| | | sul1, sul2, TEM-1D |
| | CDC 126 | aac(6′)Ib-cr, catB3, dfrA1, fosA, KPC-2, OmpK36, oqxA, oqxA, OXA-1, sul1, TEM-1B |
| | CDC 129 | aac(6′)-Ib, aadA2, aph(3′)-Ia, catAl, dfrA12, KPC-3, mph(A), OmpK35, oqxA, oqxA, oqxB, sul1, TEM-1A |
| | CDC 135 | aac(3)-IIa, aac(6′)-Ib, aph(3′)-XV, catB2, dfrA14, OmpK35, oqxA, oqxA, OXA-9, SHV-12, sul1, TEM-1A, |
| | | tet(D), VIM-1 |
| | CDC 138 | aadA2, ARR-3, CTX-M-15, dfrA12, dfrA14, mph(A), NDM-7, oqxA, oqxA, SHV-11, strA, strB, sul1, sul2, TEM-1B |
| | CDC 158 | aac(3)-IId, aac(6′)Ib-cr, CTX-M-15, dfrA14, dfrA30, fosA, NDM-1, oqxA, oqxA, oqxB, OXA-1, strA, strB, |
| | | sul2, TEM-1B, tet(B) |
| P. aeruginosa | CDC 439 | IMP |
| | CDC 444 | VIM |
| | CDC 457 | VIM |
| | CDC 356 | KPC |
| | CDC 230 | aac(3)-Id, aadA2, cmlA1, dfrB5, OXA-4, OXA-50, PAO, |
| | | tet(G), VIM-2 |
| | CDC 231 | aac(6′)-IIc, KPC-5, OXA-2, OXA-50, PAO |
| | CDC 241 | aac(6′)-IIc, aadA7, catB7, IMP-1, OXA-101, OXA-50, OXA-9, |
| | | PAO, sul1 |
| | CDC 246 | aadB, NDM-1, OXA-10, OXA-50, PAO, rmtD2, tet(G), VEB-1 |
| | CDC 250 | aadB, NDM-1, OXA-10, OXA-50, PAO, rmtD2, tet(G), VEB-1 |
| | CDC 516 | PDC-101; KPC-2 |
| | CDC 518 | PDC-103; KPC-2 |
| A. baumannii | NCTC 13304 | OXA-27 |
| | CDC 273 | aac(3)-IIa, ADC-25, aph(3′)-Ic, aph(3′)-VIa, OXA-23, OXA-66, |
| | | strA, strB, sul2 |
| | CDC 274 | aac(3)-Ia, ADC-25, aph(3′)-Ic, OXA-66, OXA-72, strA, strB, sul1, |
| | | sul2, TEM-1D |
| | CDC 275 | ADC-25, aph(3′)-Ic, armA, mph(E), msr(E), OXA-23, OXA-66, |
| | | strA, strB, sul2, TEM-1D |
| | CDC 277 | aac(3)-IIa, OXA-24, OXA-65, strA, strB, sul2, TEM-1B |
| | CDC 284 | aac(3)-IIa, OXA-24, OXA-65, strA, strB, sul2, TEM-1B |
| | CDC 307 | ADC-25, aph(3′)-Ic, armA, catB8, mph(E), msr(E), OXA-23, OXA- |
| | | 66, strA, strB, sul1, sul2 |
| | CDC 308 | ADC-25, armA, catB8, mph(E), msr(E), OXA-71, strA, strB, |
| | | sul1, TEM-1D |
| | CDC 311 | ADC-25, aph(3′)-Ic, armA, catB8, mph(E), msr(E), OXA-23, OXA- |
| | | 82, strA, strB, sul1 |
| | CDC 312 | aph(3′)-Ic, catA1, OXA-69, sul2, tet(B) |
| | CDC 313 | aac(3)-Ia, aph(3′)-Ic, catA1, OXA-23, OXA-69, TEM-1D, tet(A) |

### REFERENCES

1.) Beckman E, Wolfe C, Pillar C. In vitro Activity of Bismuth Thiols and Comparators Against Drug Resistant Gram-positive and -negative Bacteria and Yeast. Final Report Aug. 24, 2016-Microbion 22. Micromyx, Kalamazoo, MI. 2016.
2.) Clinical and Laboratory Standards Institute (CLSI). Methods for Dilution Antimicrobial Susceptibility Tests for Bacteria That Grow Aerobically; 11^{th} ed. CLSI standard M07. CLSI, 950 West Valley Road, Suite 2500, Wayne, Pennsylvania 19087 USA, 2018.
3.) CLSI. Performance Standards for Antimicrobial Susceptibility Testing; 29^{th} ed. CLSI supplement M100. CLSI, 950 West Valley Road, Suite 2500, Wayne, Pennsylvania 19087 USA, 2019.
4.) Schaadt R D, Peterson M, Sweeney D. In vitro Activity of Bismuth-1,2-ethanedithiol (BisEDT) Against Multiple Clinical Isolates of Gram-positive and -negative Bacteria. Final Report Aug. 8, 2008-Microbion 2. Micromyx, Kalamazoo, MI. 2008.

### Example 7: Activity of BT Compounds Against Biofilms

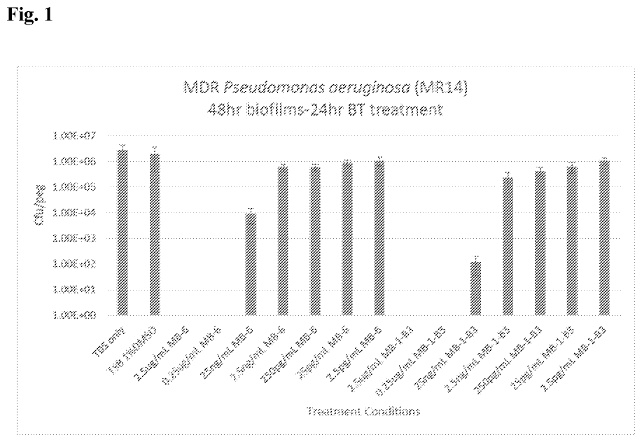

The activity of BT compounds against biofilms grown from CF-isolates was tested. MR14 is a multidrug-resistant (MDR) CF-isolate of Pseudomonas aeruginosa. Reductions in biofilm cell viability of 2 logs (MB-6) to 4 logs (MB-1-B3) occurred at 25 ng/mL (FIG. 1). The bismuth-thiol compounds have previously been reported in the literature to have anti-biofilm effects at subinhibitory concentrations with 24 hour treatment with 0.25 μg/mL. There is nothing comparable in the scientific literature.

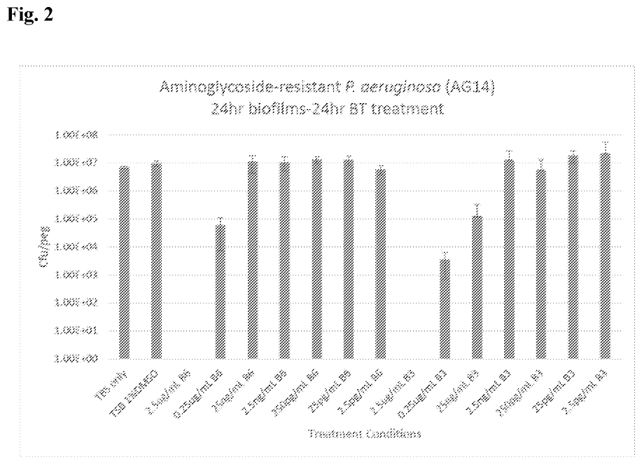

AG14 is an aminoglycoside-resistant CF-isolate of Pseudomonas aeruginosa. Reductions in biofilm cell viability of 2 logs (MB-6) to approximately 3.5 logs (MB-1-B3) occurred at 0.25 μg/mL. Once again, a very advanced level of anti-biofilm activity (a 6 log reduction) with 24 hour treatment occurred with 0.25 μg/mL; this potent level of activity is very likely to be unique to the bismuth-thiol compounds (FIG. 2).

Combined, the results of testing against Pseudomonal biofilms (MR14 and AG14) demonstrate an advanced, possibly unique level of anti-biofilm activity against antibiotic- and multidrug-resistant (MDR) Pseudomonas aeruginosa; this may represent an important new therapeutic activity and clinical strategy in the treatment of pulmonary infections associated with cystic fibrosis.

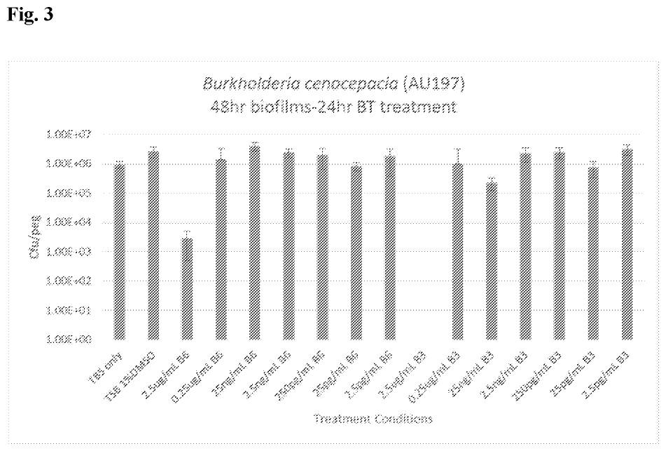

AU197 is a CF-isolate of B. cenocepacia. While in this case, the anti-biofilm activity is not occurring at a subinhibitory level (as with the previous examples of P. aeruginosa), this level of anti-biofilm activity (a 6 log reduction at a concentration of 2.5 μg/mL of BisEDT) is nevertheless extremely potent, and is very likely to be therapeutically achievable (FIG. 3).

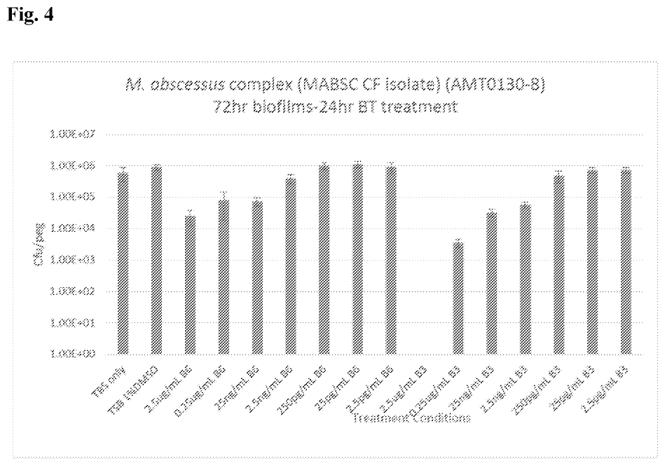

AMT0130-8 represents a CF-isolate of the clinically relevant MABSC, which frequently complicates the treatment of CF pulmonary infections. In this case, while BisBDT demonstrated only very modest reductions in biofilm cell viability, once again, BisEDT demonstrated a 6 log reduction at 2.5 μg/mL, as well as a dose response from 2.5 ng/mL to 2.5 μg/mL. Interestingly, the MIC against this strain was demonstrated to be lower for BisBDT (0.0625 μg/mL) than for BisEDT (0.125 μg/mL), yet the anti-biofilm activity of BisEDT was apparently demonstrated to be much more potent—while it is not surprising to see such differences in activity between distinct bismuth-thiol compounds, this particular MABSC strain was apparently technically difficult to work with (see bullet point notes below figure), which may also have accounted for such (apparent) differences in activity (FIG. 4).

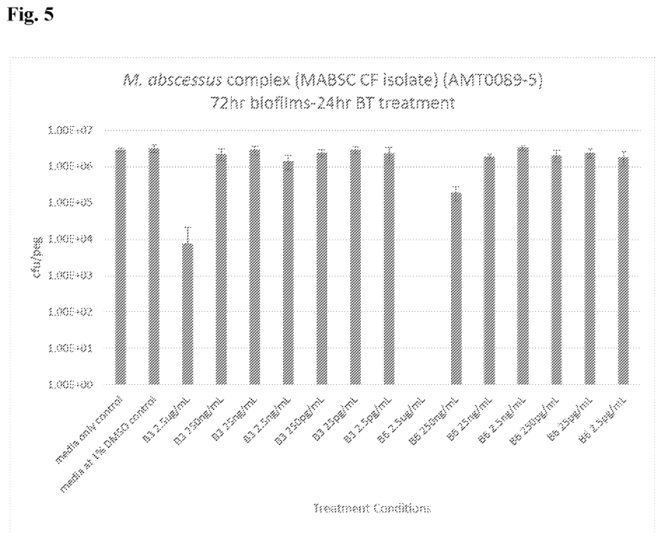

AMT0089-5 is a macrolide-resistant, amikacin-resistant MABSC. The involvement of such antibiotic-resistant strains of MABSC in the pulmonary infections of CF patients is extremely problematic. Here, while BisEDT showed a 2.5 log reduction in viable biofilm cells at a concentration of 2.5 μg/mL, BisBDT was demonstrated to have reduce viable biofilm cells by 6 logs at a concentration of 2.5 μg/mL (FIG. 5).

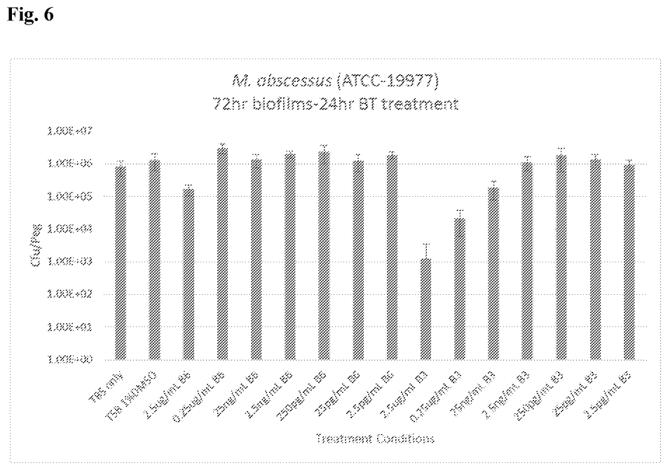

ATCC-19977 is M. abscessus (macrolide-resistant; inducible). A dose response is demonstrated showing a 3 log reduction in viable biofilm cells at 2.5 μg/mL ATCC-19977 is M. abscessus (macrolide-resistant; inducible). A dose response is demonstrated below, with a 3 log reduction in viable biofilm cells at 2.5 μg/mL (FIG. 6).

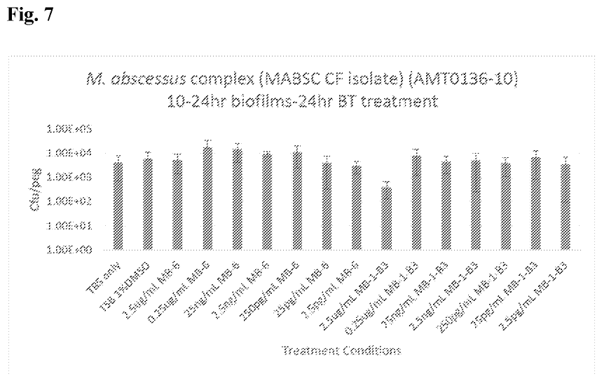

The bismuth-thiols were not observed to be active against biofilm formed by a MABSC CF isolate, though this strain was so slow-growing, a longer exposure to the bismuth-thiol compounds may have been necessary to demonstrate activity (FIG. 7).

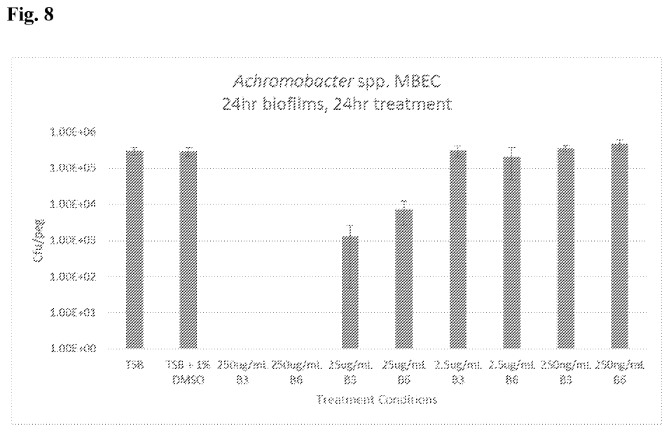

Achromobacter spp. were tested up to concentrations of 250 μg/mL of both BisEDT and BisBDT, which resulted in a 5 log reduction in viable biofilm cells. A dose response is also apparently associated with both compounds (FIG. 8).

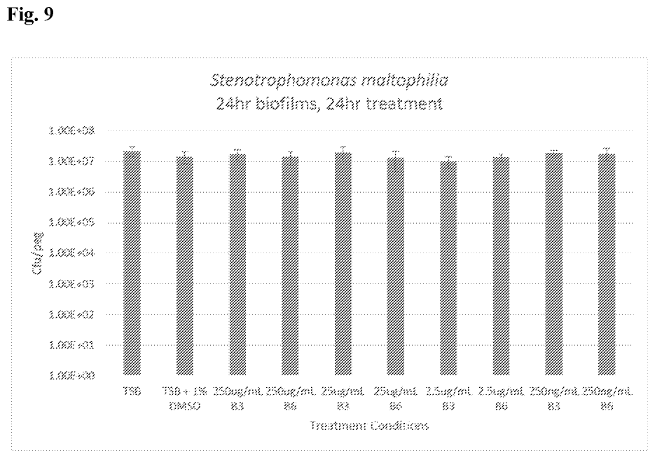

Unfortunately, no activity was apparent for either bismuth-thiol compound against Stenotrophomonas maltophilia (FIG. 9).

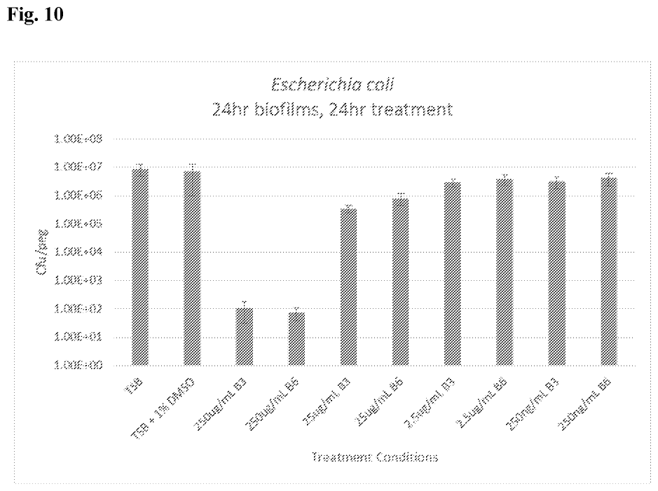

Finally, anti-biofilm activity was also demonstrated at the highest concentrations of both compounds (a 5 log reduction) when tested against E. coli (FIG. 10).

Both BisEDT and BisBDT are demonstrated to have very low MIC values against M. abscessus, MDR P. aeruginosa, Achromobacter spp., and Burkholderia spp. As before, ATCC control strains are utilized to standardize the data. However, in this evaluation, the bismuth-thiols were compared head to head with amikacin and clarithromycin (a clinically important macrolide antibiotic). As can be seen from this data below in Tables 18 and 19, the bismuth-thiols are notably and consistently more potent than both amikacin and clarithromycin (most dramatically when considering the MABSC strain ATCC 19977, which was induced to be macrolide-resistant).

TABLE 18
Comparison of conventional antibiotics vs BisEDT and BisBDT activity against bacterial strains
SUMMARY OF MIC RESULTS

| | | Special Strain | MIC | MIC | MIC | MIC |
|---|---|---|---|---|---|---|
| Strain | | characteristics | (mcg/mL) | (mcg/mL) | (mcg/mL) | (mcg/mL) |
| Designation | Species | (if any) | Amikacin | Clarithromycin | Bis-EDT | Bis-EDT |
| ATCC 19977 | M abscessus/massiliense complex | Macrolide resistant | 8² | >32² | 0.06² | <0.03² |
| | | (inducible) | | | | |
| AMT0130-8 | M abscessus/massiliense complex | | 16² | 1 | 0.06² | <0.03² |
| AMT153-9 | M abscessus/massiliense complex | | 32² | 2 | 0.13² | <0.03² |
| AMT0068-40 | M abscessus/massiliense complex | | 32² | 1 | 0.25² | 0.06² |
| AMT0119-7 | M abscessus/massiliense complex | | 32² | 1 | 0.06² | <0.03² |
| AMT0493-2 | M abscessus/massiliense complex | Amikacin resistant | >64² | 2 | 0.5² | 0.125² |
| AGR1 | P. aeruginosa | | 16 | na | 1 | 1 |
| AGR14 | P. aeruginosa | Multi-drug resistant | >64 | na | 0.5 | 1 |
| MR14 | P. aeruginosa | Multi-drug resistant | >64 | na | 1 | 1 |
| SM21 | S. maltophilia | | 64 | na | 0.25 | 0.13 |
| AX1 | Achromobacter spp. | | 64 | na | 1 | 1 |
| AX4 | A. xylosoxidans | | >64 | na | 0.25 | 0.5 |
| BC5b | B. multivorans (B cepacia complex) | | >64 | na | 0.25 | 1 |
| BC15 | B. cenocepacia (B cepacia complex) | | >64 | na | 2 | 4 |
| BC17 | B. cepacia (B cepacia complex) | | >64 | na | 8 | 8 |
| AU197 | B. cenocepacia (B cepacia complex) | | >64 | na | 0.5 | 4 |

TABLE 19
BisEDT and BisBDT MIC against control bacterial strains

| ATCC or other strains used to control for drug activity | | Expected results (acceptable range) mcg/mL (per CLSI M100-S24 or | | | |
|---|---|---|---|---|---|
| (see detailed data for measured MICs with each assay set-up) | | provided by Microbion) | | | |
| Strain | Species | Amikacin | Clarithromycin | BisEDT | BisBDT |
| ATCC 29213 | S. aureus | 2 (1-4) | 0.25 (0.12-0.5) | 0.25-0.5 (0.13-1) | 0.25 (0.12-0.5) |
| ATCC 25922 | E. coli | 1-2 (0.5-4) | na | 1 (0.5-2) | na |

### Example 8: Clinical Evaluation of BisEDT for Treating Osteomyelitis

Study: A Phase 2a Randomized, Single-Blind, Placebo-Controlled, 12-week Escalating Dose Study to Assess the Safety, Tolerability and Clinical Activity of 3 Concentrations of Locally Applied MBN-101 to Infected Osteosynthesis or Osteomyelitis Bone Sites.

Objective: To evaluate the potential of BisEDT, the broad-spectrum antimicrobial/antibiofilm agent in MBN-101, in promoting more rapid/complete resolution of orthopaedic infections.

### Investigational Therapy:

MBN-101 [Bismuth-1,2-ethanedithiol (BisEDT) sterile, aqueous suspension,] represents the first drug product from a new class of antimicrobial agents with unique mechanisms of action. In nonclinical models, BisEDT has been shown to be effective against a broad-spectrum of orthopaedic device-associated bacteria including antibiotic-resistant strains.

BisEDT has several characteristics especially suited to the treatment of postoperative orthopaedic infections:
(a) Broad-spectrum antimicrobial activity against the most critically important antibiotic-resistant bacteria, including MRSA, methicillin-resistant Staphylococcus epidermidis, antibiotic-resistant Pseudomonas aeruginosa, extended spectrum beta lactamase-positive Klebsiella pneumoniae, and antibiotic-resistant Enterobacter species.
(b) Unique mechanism of action (MOA), with no known instances of cross-resistance. Mechanism of action is the reversible inhibition of bacterial membrane potential, causing global inhibition of all major macromolecular biosynthetic pathways including DNA, RNA, protein, and lipid synthesis.
(c) Highly probable relationship between MOA and anti-biofilm capabilities.
(d) Highly probable relationship between MOA and inability to experimentally generate any stable resistant bacterial mutant to BisEDT after repeated spontaneous mutation frequency studies, and repeated serial passage studies.
(e) Efficacy against both aerobic and anaerobic bacteria.
(f) Demonstrated lack of interference with the activity of a wide range of antibiotics.
(g) Enhanced activity in combination with specific antibiotics; in some cases, synergy has been demonstrated in vitro and in vivo.
(h) Maintenance of antimicrobial activity in the presence of excessive protein.
(i) A favorable safety and tolerability profile in nonclinical toxicology studies in a variety of species, including absence of deleterious effects on wound healing or bone repair.
(j) Favorable safety and tolerability profiles in clinical Phase 1 studies evaluating topical administration.

Based in part on these characteristics, MBN-101 has been granted Qualified Infectious Disease Product and Fast Track designations by the Food and Drug Administration (FDA) for the local, intra-operative treatment of resistant post-surgical orthopaedic implant infections.

### Advantages of MBN-101 for Treatment of Infections:

The broad-spectrum antimicrobial, anti-biofilm activity of BisEDT (the active pharmaceutical ingredient in MBN-101), its activity against relevant antibiotic-resistant pathogens, and its ability to enhance the activity of certain other antibiotics are properties that promote more rapid and/or more complete eradication of infection and reduce infectious risks to patients. With comparatively elevated rates of both postoperative infection associated with repair of traumatic orthopaedic wounds, and antibiotic-resistance associated with osteomyelitis, the development of a new and innovative treatment strategy to complement the current standard of care would be expected to result in a substantial reduction in mortality, amputation, morbidity, and disability, along with a reduction in patient treatment costs.

MBN-101 provides important advantages over current standard of care treatment for orthopaedic infections. Direct, local contact of MBN-101 with infected target tissue and contaminated device surfaces immediately delivers a therapeutically active dose of BisEDT to the site of infection. Combined antimicrobial and anti-biofilm effects are achieved with minimal systemic exposure (see PK results below). Systemic antibiotics administered alone are frequently ineffective, in part because altered perfusion at the surgical wound site hinders effective and timely delivery of systemic antibiotics, making it difficult to reach therapeutic antibiotic levels at wound tissues/surfaces. The combined effect of intravenously administered antibiotics, which already serve as a pillar of the current standard of care for orthopaedic device-related infections, along with the local administration of MBN-101, is expected to eradicate bacteria and their associated biofilm from postoperative orthopaedic wounds, and will ultimately also serve to reduce the likelihood of development of antibiotic-resistant bacterial infection. By improving the ability of current antibiotic therapies to effectively control and eliminate post-surgical orthopaedic device-related infections caused by both aerobic and anaerobic pathogens, as well as reducing the time to resolution of infection, MBN-101 will contribute to reductions in the number of additional serious interventions needed to resolve infections, including reduction in repeat surgeries, additional courses of systemic antibiotics, patient hospitalization time, and patient morbidity and mortality in both civilian and military populations.

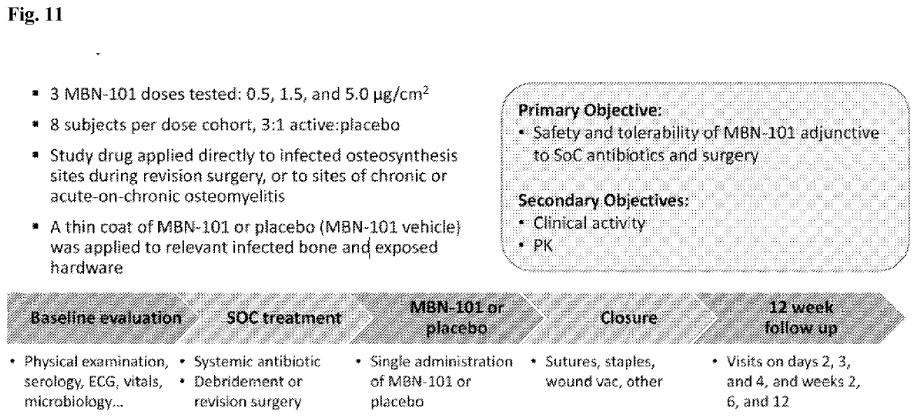

### Study Design (FIG. 11):

This was a randomized, single-blind, placebo-controlled, multicenter study to assess the safety and tolerability of single escalating doses of MBN-101 applied directly to target structures within infected osteosynthesis sites during revision surgery, with or without hardware removal and replacement for subjects diagnosed with an apparent fracture site infection, or to sites of chronic or acute-on-chronic osteomyelitis of the long bone extremities or residual amputated limbs. The study was registered in ClinicalTrials.gov (NCT #02436876) and was subject to review and approval by local Institutional Review Boards at each clinical site prior to enrollment of subjects.

Three successive cohorts of 8 subjects each were enrolled in this study. Consecutive subjects from each of the study sites were screened for potential participation as they presented to the orthopaedic service for clinical care for their infections. After signing an informed consent form (ICF), subjects completed screening procedures. Subjects who met the eligibility criteria at Screening were offered participation in the study.

Subjects were randomized in a 3:1 ratio to receive study drug (MBN-101 or placebo). Subjects were enrolled in 3 consecutive dose cohorts and received 0.025, 0.075, or 0.25 mg/mL weight:volume (w:v) of BisEDT formulated as MBN-101 (equal to 0.5, 1.5, and 5.0 μg/cm² BisEDT, respectively, and up to 8 mL, for a dose of up to 0.2, 0.6, or 2.0 mg BisEDT, respectively) or placebo (diluent). Enrollment of the next cohort did not commence until the Data Review Committee (DRC) reviewed all available safety data on all subjects through Week 6 of the study and approved escalation to the next cohort. The DRC monitored all safety data in an ongoing manner from all subjects enrolled into this study.

Following the baseline evaluation, subjects received standard of care treatment for their infected bone site, including postoperative fracture site infection and osteomyelitis, that included systemic antibacterial treatment per institutional standard of care guidelines and debridement/revision surgery with or without hardware removal and placement/replacement as indicated. Multiple debridements, soft tissue transfer, and revision fixation procedures may have been performed prior to definitive closure. A single application of study drug, MBN-101 or placebo, applied intra-operatively directly to target structures within infected bone sites, was performed following the final irrigation and debridement procedure and immediately prior to definitive closure of the surgical wound. In cases where original hardware was retained, study drug was sparingly applied to all of the accessible surfaces of the hardware and adjacent bone. In cases where the hardware was placed or replaced, or in cases of 2-stage procedures, study drug was applied to all of the accessible surfaces of the hardware and adjacent bone following implantation of hardware and immediately prior to definitive closure of the surgical wound. In cases where hardware was not required, study drug was applied to the affected areas of the bone only prior to definitive closure. The volume applied was determined by the surgeon's assessment of the size (in cm²) of the target area. The minimum amount of study drug required to achieve a thin coat of the relevant target structures within the infected bone site was used.

All subjects received standard postoperative care per institutional guidelines and were discharged from the hospital in accordance with local standards. A single application of study drug was applied topically on Day 1 (baseline); subjects underwent subsequent study visits on Days 2, 3, and 4, and at Weeks 2, 6, and 12. All subjects were to be followed for a minimum of 12 weeks after surgery or until early termination. Per the protocol in effect at the time, 5 subjects in the 0.5 μg/cm² dose group and 1 subject in the placebo group participated in the study for 24 weeks.

Blood samples for a planned PK analysis of bismuth (Bi) as a surrogate for Bismuth-1,2-ethanedithiol were collected predose and at nominal 1, 6, 12, 24, 36, 48 (Day 2), 60, 72 (Day 3), 96 (Day 4), and 336 (week 2) hours postdose. Subject-reported outcomes were collected using the Veterans RAND 12-item Health Survey (VR-12) and Short Musculoskeletal Function Assessment (SMFA) at Screening and at Weeks 2, 6, and 12. A surgical site pain score was collected by Visual Analog Score (VAS) on Day 1 and at all subsequent visits. The surgical site was examined for local erythema, induration, drainage, and degree of healing on Day 1 and at all subsequent visits. Radiographic evaluations were performed on Day 1 and at Weeks 2, 6, and 12.

Safety assessments included adverse events; clinical laboratory tests (including clinical chemistry, hematology, and urinalysis); vital signs; physical examinations; electrocardiograms (ECGs); and microbiology status of the index site.

Duration of treatment: A single treatment was administered on a single day.

### Objectives:

The primary objective of the study was to evaluate the safety and tolerability of single escalating doses of locally administered MBN-101 or placebo as adjunct to standard of care antimicrobial and surgical therapy in patients with orthopaedic infections.

Secondary objectives included evaluation of the clinical activity and the pharmacokinetics (PK) of single escalating doses of locally administered MBN-101.

### Inclusion Criteria:

To be eligible for this study, each of the following criteria must be satisfied with a “Yes” answer:
1. subjects (male of female) ≥18 and ≤75 years of age must have had operative fracture fixation of the upper extremity, lower extremity or pelvis, or had undergone arthrodesis and subsequently were diagnosed with an apparent fracture site infection or were diagnosed with chronic or acute-on-chronic osteomyelitis of the long bone extremities. Subjects also had to have required surgical debridement of infected soft tissue and/or bone, with or without removal and/or placement/replacement of hardware, and had at least 1 of the following:
   a) Elevated erythrocyte sedimentation rate (ESR) above the upper limit of normal (ULN)
   b) Elevated C-reactive protein (CRP) above the ULN
   c) Draining wound/sinus tract
   d) Positive culture from site of prior surgery by aspirate or other modality
   e) Local erythema or induration at the site of prior surgery
   f) Exposed hardware
   g) Periosteal reaction on x-ray
   h) Loose or broken hardware;
      and require surgical debridement of infected soft tissue and/or bone, with or without removal and/or placement/replacement of hardware
2. Patients receiving or anticipated to receive systemic antibiotic therapy as per institution's standard of care
3. Patients requiring postoperative hospitalization for at least 48 hours after surgery

### Exclusion Criteria:

1. Patients with multiple, non-contiguous sites of infection
2. Pathologic fracture (not including osteoporosis)
3. Patient requires immunosuppressive therapy (Topical or inhaled corticosteroids are permitted)
4. Serum creatinine, alanine transaminase, aspartate transaminase, or alkaline phosphatase>2.0 times the ULN range of the local testing laboratory
5. Absolute neutrophil count<1000
6. Patients without definitive soft-tissue coverage over the surgical site at time of study product administration
7. Any condition that has required treatment with any other Bi-containing compound within the last 2 weeks (i.e., Kaopectate or Pepto-Bismol)
8. Participation in an investigational trial to evaluate pharmaceuticals or biologics within the past 3 months
9. Individuals undergoing surgical treatment for more than 1 infected site
10. Known allergy to metals or materials comprising the orthopedic hardware, Bi and/or MBN-101 excipients (methylcellulose, Tween 80 (polysorbate 80))
11. Patients who are pregnant, lactating, or female patients, who have a positive serum human chorionic gonadotropin as determined by laboratory testing
12. Immunocompromised due to illness or organ transplant
13. History of chronic or recurrent infections (≥3 infections at the same site within 12 months) other than the index infected bone site
14. History of any type of cancer (excluding non-melanomatous localized skin cancer or completely excised and cured carcinoma-in-situ of uterine cervix)
15. Poorly controlled diabetes mellitus (hemoglobin Ale>9.0% for ≥6 months despite management by a physician)
16. History of medical noncompliance
17. Other medical conditions which, in the opinion of the PI, would jeopardize the safety of the study subject or impact the validity of the study results

### Investigational Product:

BisEDT, the active agent in MBN-101 was prepared according to the protocol described in Example 4.

Subjects received a single dose of 0.025, 0.07.5, or 0.2.5 mg/mL (w:v) of MBN-101 (equal to 0.5, 1.5, and 5.0 μg/cm² BisEDT, respectively, and up to 8 mL, for a dose of up to 0.2, 0.6, or 2.0 mg BisEDT, respectively) or placebo (diluent).

MBN-101 was diluted by the pharmacy and provided to the surgeon as BisEDT suspension (0.025, 0.075, or 0.25 mg/mL, w:v]) in diluent (3% methylcellulose/0.5% Tween 80/10 mM sodium chloride/10 mM sodium phosphate, pH 7.4). A stock formulation contained 2.5 mL of sterile 2.5 mg/mL MBN-101. The lot number was MB399-1-15-01.

Placebo was provided as MBN-101 diluent comprised of 3% methylcellulose/0.5% Tween 80/10 mM sodium chloride/10 mM sodium phosphate, pH 7.4. The lot number for Cohort 1 was MD402-1-15-01 and for Cohort 2 and Cohort 3 was MD402A02.

The treatment dose for each subject was prepared at the clinical site under sterile conditions no more than 8 hours prior to dose administration using the Stock Formulation and Diluent provided in the Drug Product Kit according to the directions-for-use (DFU) provided. The treatment dose was provided to the surgeon in a vial from which an 8 mL volume was drawn up in the operating room into a sterile 10 mL syringe. Surgeons were instructed to use sufficient volume (up to the full 8 mL provided for the largest exposed target areas) to sparingly coat the bone at the site of infection, the exposed surfaces of any retained hardware, and the surfaces of any new hardware to be implanted. The volume of MBN-101 required to achieve a thin coat of the relevant structures within the infected bone site was applied. See Table 20 for the recommended volume of MBN-101 based on area of infected bone site.

TABLE 20
Recommended Volume of MBN-101 Based
on Area of Infected Bone Site

| | |
|---|---|
| Area of Infected Bone Site | Recommended Volume of MBN-101 |

| | | |
|---|---|---|
| 25 | cm² | 0.5 mL |
| 50 | cm² | 1.0 mL |
| 75 | cm² | 1.5 mL |
| 100 | cm² | 2.0 mL |
| 125 | cm² | 2.5 mL |
| 150 | cm² | 3.0 mL |
| 175 | cm² | 3.5 mL |
| 200 | cm² | 4.0 mL |
| 225 | cm² | 4.5 mL |
| 250 | cm² | 5.0 mL |
| 275 | cm² | 5.5 mL |
| 300 | cm² | 6.0 mL |
| 325 | cm² | 6.5 mL |
| 350 | cm² | 7.0 mL |
| 375 | cm² | 7.5 mL |
| 400 | cm² | 8.0 mL |

Source: Study Protocol (Appendix 16.1.1)

### Dose Selection

Three successive doses (0.025, 0.075, or 0.25 mg/mL [w:v] BisEDT) of MBN-101 (equal to 0.5, 1.5, and 5.0 μg/cm² BisEDT, respectively, and up to 8 mL, for a dose of up to 0.2, 0.6, or 2.0 mg BisEDT, respectively) or placebo (diluent) were studied. In all cases, the minimum amount of MBN-101 required to coat the relevant structures within the infected site was used. The suspension was applied in a thin layer to cover all affected areas of bone as well as any exposed hardware. Direct application to muscle and other soft tissues was avoided. The approximate area of the infected site was calculated based on the length of exposed bone multiplied by the width of exposed bone: the surface area of the hardware was added to this value to derive the approximate area of the infected site. Recommended maximum volumes of MBN-101 for various areas of the infected site are provided in Table 20. For infected bone surface areas that fell between the specified areas, the applied volume was rounded up to the volume for the next area specified in the table (e.g., an 80 cm² wound would have been rounded up to 100 cm², and the volume of MBN-101 administered would have been 2.0 mL).

By following the application requirements in Table 20, the administered doses were:
Cohort 1: 0.025 mg/mL MBN-101=0.5 μg/cm²
Cohort 2: 0.075 mg/mL MBN-101=1.5 μg/cm²
Cohort 3: 0.25 mg/mL MBN-101=5.0 μg/cm²

TABLE 21
Schedule of procedures

| | | Baseline/ | | | | | | Week 12, | Early |
|---|---|---|---|---|---|---|---|---|---|
| | | Day of | | | | Week 2 | Week 6 | EOT | Term/ |
| | | Surgery | Hour 48-60 | Hour72 | Hour 96 | Day 14 ± | Day 42 ± | Day 84 ± | Tx |
| Procedure | Screening | Day 1 | Day 2 | Day 3 | Day 4 | 4 days | 8 days | 8 days | Failure |
| Review of eligibility criteria | X | X | | | | | | | |
| Inferred consent | X | | | | | | | | |
| Randomization | | X | | | | | | | |
| Surgey/administration of study | | X | | | | | | | |
| drug | | | | | | | | | |
| Medical history | X | | | | | | | | |
| Physical examination | X | | | | | | | | |
| Interval physical examination | | X | X | | | X | X | X | X |
| Hematology | X | | X | | | | X | X | X |
| Serology | | X | | | | X | X | X | X |
| Serum chemistry | X | | X | | | | X | X | X |
| Urinalysis | X | | X | | | | X | X | X |
| Pregnancy test | X | X | | | | | | X | X |
| BisEDT blood levels¹ | | X | X | X | X | X | | | |
| 12-lead ECG | X | X | X | | | | | X | X |
| Vital signs | X | X | X | X | X | X | X | X | X |
| Subject-reported outcomes² | X | | | | | X | X | X | X |
| Surgical site signs and symptoms | | X | X | X | X | X | X | X | X |
| Surgical site pain score | | X | X | X | X | X | X | X | X |
| Microbiology | | X | X³ | | | X³ | X³ | X³ | X³ |
| Radiographic evaluation | | X | | | | X | X | X | X |
| Concomitant medications | X | X | X | X | X | X | X | X | X |
| Advers events | | X | X | X | X | X | X | X | X |

¹Pharmacokinetic blood samples were collected predose and at nominal 1, 6, 12, 24, 35, 48 (Day 2), 60, 72 (Day 3), 96 (Day 4), and 336 (Day 14, Week 2) hours after administration of study drug
²Subject-reported outcomes included the Veterans RAND 12-Item Health Survey and Short Musculoskeletal Function Assessment.
³Microbiology was performed at any time that a new surgical intervention was required.
BisEDT = Bismuth-1,2-ethanedithiol; ECG = electrocardiogram; EOT = End of Treatment; Term = termination; Tx = treatment.
Source: Study Protocol (Appendix 16.1.1)

### Efficacy and Safety Variables:

Table 21 presents the Schedule of Procedures performed during the study.

A comprehensive medical history (including allergic and psychosocial history) was obtained at Screening.

A comprehensive physical examination including height, weight, head, ears, eyes, nose, throat, chest, heart, abdomen, and skin was performed at Screening. An interval physical examination that focused on assessing local erythema, induration, drainage, and degree of healing of the surgical site was performed on Days 1 and 2, and at weeks 2, 6, and 12. Cumulative number of serious interventions at Week 12.

Clinical laboratory tests (including clinical chemistry, hematology, and urinalysis) were performed at Screening, on Day 2, and at weeks 6 and 12.

Serology tests for ESR and CRP were performed on Day 1 and at Weeks 2, 6, and 12.

Serum pregnancy tests for women of childbearing potential were performed at Screening, on Day 1, and at Week 12.

Blood samples for a planned PK analysis of Bi as a surrogate for BisEDT were collected predose and at nominal 1, 6, 12, 24, 36, 48 (Day 2), 60, 72 (Day 3), 96 (Day 4), and 336 (Week 2 visit) hours postdose.

Twelve-lead ECGs were recorded at Screening, on Days 1 and 2, and at Week 12.

Vital signs (including blood pressure, pulse, respiratory rate, and body temperature) were recorded at Screening and at all subsequent visits.

Subject-reported outcomes were collected using the VR-12 and SMFA at Screening and at Weeks 2, 6, and 12.
The VR-12 was a self-administered health survey consisting of 12 items corresponding to 8 principal physical and mental health domains including general health perceptions; physical functioning; role limitations due to physical and emotional problems; bodily pain; and energy fatigue, social functioning, and mental health.
The SMFA questionnaire consisted of the dysfunction index with 34 items and the bother index with 12 items. The dysfunction index was grouped into 4 categories (daily activities, emotional status, function of the arm and hand, and mobility) and assessed the subject's perceptions of the amount of difficulty they had in the performance of certain functions (2.5 items) and how often the subjects had difficulty when performing certain functions (9 items). Each item had a 5-point response format (1 point for good function and 5 points for poor function). The bother index asked the subjects to assess how much they were bothered by problems in various areas of life (e.g., recreation, work, sleep, and rest). These items also had a 5-point response format (1 point for not at ail bothered and 5 points for extremely bothered). The scores of the dysfunction and the bother indices were calculated by summing up the responses to the items and then transforming the scores to give the final scores, which ranged from 0 to 100.

Surgical site signs and symptoms were evaluated on Day 1 and at all subsequent visits. The surgical site was examined for local erythema, induration, drainage, and degree of healing. Subjects with non-healing or worsening status of their surgical site may have been considered for additional standard of care treatment but were encouraged to remain in the study in order to complete study evaluations.

Surgical site pain score was assessed on Day 1 and at all subsequent visits. Pain related specifically to the surgical site was assessed by VAS utilizing a 24-hour recall. The assessment was based on a 0 to 10 scale, where 0=no pain at all and 10=the worst possible pain.

Radiographic evaluation was to be performed on Day 1 and at Weeks 2, 6, and 12. At least 2 orthogonal views were reviewed for bone morphology and integrity, periosteal reaction, union, interval callus formation, loss or change in reduction, and hardware integrity/failure.

Concomitant medications were recorded at Screening and at all subsequent visits.

Adverse events were recorded on Day 1 and at all subsequent visits.

### Microbiology

Microbiology was assessed on Day 1 and at the time of any subsequent surgical procedure at the index site. Samples were collected from the infected bone site or involved tissue adjacent to any implant(s), swabs of the deep infection site, and the superficial tissue/wound closure/sinus tract site. Isolation and identification of aerobic and anerobic bacteria was conducted at a central microbiology laboratory (IHMA, Schaumburg, IL) and susceptibility of the isolates to MBN-101 (BisEDT) and comparator agents was performed per Clinical and Laboratory Standards Institute (CLSI). Parallel sets of samples were analyzed by the local clinical site laboratory to guide patient antibiotic treatment.

### Incidence of Serious Interventions

All interventions meeting the following criteria were serious interventions:
Initiation of antibiotic treatment in subjects not previously on antibiotics.
Change in the route of antibiotic administration from oral to intravenous for escalation of treatment.
Change in the type or dosage of antibiotic drug for escalation of treatment.
Readmission (exclusive of readmissions associated with a healed bone site)
Reoperation (exclusive of reoperations associated with a healed bone site).

### Efficacy Variables and Assessment

Clinical activity of locally administered MBN-101 was assessed by the following:
Proportion of treatment failures. A treatment failure was defined as a subject with non-healing or worsening status of their surgical site requiring serious intervention by Week 12
Cumulative number of serious interventions at Week 12.
Time to first serious intervention, time to readmission, and time to reoperation, exclusive of serious interventions, readmissions, and reoperations associated with a healed bone site.
Number of subjects undergoing removal of stabilizing orthopaedic hardware due to a healed bone site by Week 12.
Time to removal of orthopaedic hardware due to a healed bone site.
Incidence and intensity of surgical site signs and symptoms, including local erythema, induration, drainage, and degree of healing.
Change from baseline in subject-reported outcome.
Surgical site pain score and change from baseline in pain score at each post-baseline time point. Pain intensity at the surgical site was assessed by VAS (0 to 10 scale).
Findings of microbiology evaluations at the surgical site. Microbiologic success was assessed by clearance of infection.
Findings of radiographic evaluations at the surgical site. Radiographic success was assessed by healing on radiologic examination.
Change in serologic markers CRP and ESR at each post-baseline time point.

### Pharmacokinetic Assessment

Whole blood concentrations of Bi (Bi as a surrogate for BisEDT) were measured after administration of a single dose of MBN-101. Calculation of the following pharmacokinetic parameters was planned if exposure levels were adequate:
Time to maximum observed concentration of Bi.
Maximum observed concentration of Bi.
Area under the Bi concentration versus (vs) time curve from time zero to the last measurable concentration.
Area under the Bi concentration vs time curve from time zero and extrapolated to infinity.
The apparent half-life of Bi after extravascular administration of BisEDT.
Apparent clearance after extravascular administration of BisEDT
Apparent volume of distribution after extravascular administration of BisEDT.

Blood samples for a planned PK analysis of bismuth (Bi) as a surrogate for BisEDT were collected predose and at nominal 1, 6, 12, 24, 36, 48 (Day 2), 60, 72 (Day 3), 96 (Day 4), and 336 (Week 2) hours postdose. BisEDT levels in whole blood after MBN-101 administration were assessed with a validated inductively coupled plasma mass spectrometry assay method using Bi as a surrogate for BisEDT with a lower limit of detection of 0.5 ng/mL.

### Safety Variables and Assessment

Safety variables included incidence, intensity, and relatedness of treatment-emergent adverse events (TEAEs) and treatment-emergent serious adverse events (SAEs); changes in clinical laboratory parameters (clinical chemistry, hematology, and urinalysis); changes in vital signs (blood pressure, pulse, respiratory rate, and body temperature); changes in physical examinations; clinical findings of 12-lead ECG; and changes in microbiology status.

### Statistical Methods:

Summary statistics are presented by treatment group. For continuous variables, the number of observations (n), mean, standard deviation (SD), median, minimum, and maximum are provided. For categorical variables, the frequency and percentage in each category are displayed.

For summary statistics, the mean and median are displayed to 1 decimal place greater than the original value and the measure of variability (e.g., SD) is displayed to 2 decimal places greater than the original value. All analyses were performed using SAS@ Version 9.3.

All safety and efficacy endpoints are tabulated with descriptive statistics; data from all placebo subjects (2 subjects per cohort were planned; 7 subjects were enrolled in total) were pooled. Data from MBN-101—treated subjects are presented by dose (6 subjects per dose) and combined (a total of 18 subjects). When data permitted, differences between the treatment groups and 95% confidence intervals (CIs) for the difference were presented.

In the event that a subject received study drug that was not the assigned/randomized treatment group, the subject had a major protocol deviation. The subject, however, was to be included in the analysis for safety and efficacy in the actual treatment group received.

### Sensitivity Analysis of Treatment Failure

The number and percent of subjects with treatment failure during the study are tabulated by treatment group.

### Incidence of Serious Intervention, Readmission, and Reoperation

The number and percent of subjects with at least 1 serious intervention, readmission, and reoperation (exclusive of serious interventions, readmissions, and reoperations associated with a healed bone site) and the total number of serious interventions, readmissions, and reoperations are tabulated by treatment group for the duration of the study, within the first 4 weeks after surgery, from Week 4 to Week 8, and from Week 8 to Week 12. Time to the first serious intervention, time to readmission, time to reoperations, and time to removal of hardware are summarized descriptively using the Kaplan-Meier estimator by treatment group.

The number and percentage of subjects with surgical site signs and symptoms including local erythema, induration, drainage, and degree of healing are summarized descriptively by treatment group at baseline and each scheduled post-baseline time point. The intensity of the signs and symptoms were also summarized.

### Self-Reported Outcomes

Presentation of the VR-12 total score was planned, but the calculation was not performed due to analysis limitations of the instrument that was obtained. The physical health domain score (PCS) and mental health domain score (MCS) at each scheduled visit and change from baseline are summarized descriptively by treatment group.

The SMFA results included the scores of the dysfunction and bother indices, which were calculated by summing up the responses to the items and then transforming the scores according to the formula: (actual raw score−lowest possible raw score)/(possible range of raw score)×100. The SMFA results and change from baseline are summarized by visit and treatment group with descriptive statistics.

### Other Assessments

The surgical site pain score by VAS and change from baseline are summarized descriptively by treatment group at baseline and each scheduled post-baseline time point.

The number and percentage of subjects with microbiological success are tabulated descriptively by treatment group and time point.

The number and percentage of subjects with radiographic success are tabulated descriptively by treatment group and time point.

Serologic marker CRP and ESR values and the change from baseline for each scheduled visit are summarized with descriptive statistics by treatment group. The number and percentage of subjects with normal, abnormal clinically significant, and abnormal not clinically significant for each serologic marker are tabulated by treatment group at each scheduled visit.

### Subgroup Analysis for Treatment Failure

If data permitted, the following selected subgroups were to be performed for subgroup analysis of treatment failure.
1. Area of the infected site
2. Method of fracture fixation
3. Type of infection (per identified microorganism)
4. Anatomic location of the surgery site
5. Hardware retention status (retained versus exchanged)
6. Type of injuries
7. Method of wound closure
8. Osteomyelitis versus non-osteomyelitis

### Adverse Events

A TEAE was defined as any adverse event starting after the first dose of study dmg was administered. If the adverse event was present prior to the administration of the first dose of study drug but increased in severity, it was also considered a TEAE.

An overview of adverse events is provided, which summarizes the number and percentage of subjects and the number of events for all TEAEs, drug-related TEAEs, maximum severity of TEAEs, deaths, SAEs, and discontinuations due to adverse events.

The number and percentage of subjects with at least 1 TEAE are presented by SOC and preferred term. Drug-related TEAEs, all SAEs, all adverse events leading to study discontinuation, and pre-specified local TEAEs are summarized in the same manner. In the case of multiple occurrences of the same adverse event within the same subject, each subject was counted only once for each SOC and preferred term.

Summaries are provided by maximum severity and relationship to study drug for the number and percentage of subjects with TEAEs by SOC and preferred term. Maximum severity for pre-specified local TEAEs are summarized in the same manner. For this summary, subjects with multiple adverse events were counted only once by the maximum severity within an SOC and preferred term.

Subject listings of SAEs and of adverse events causing discontinuation of the study are provided. All adverse events are listed.

### Pharmacokinetics

BisEDT levels in whole blood after MBN-101 administration were assessed with a validated inductively coupled plasma mass spectrometry assay method using Bi as a surrogate for BisEDT according to the schedule.

Summary statistics (N, mean, standard deviation, minimum, median, maximum, percent coefficient of variability) for the PK concentration data and PK parameters were to be presented by treatment group and time point. A listing of the PK concentrations by subject is provided.

### Clinical Laboratory Evaluations

Laboratory test results (clinical chemistry and hematology) at each scheduled visit and change from baseline are summarized by treatment group.

Laboratory data are tabulated using counts and percentages based on the result class (normal or abnormal) by each scheduled visit and treatment group.

The number and percentage of subjects with potentially clinically significant abnormal liver function tests are summarized.
Alanine transaminase≥3×ULN.
Aspartate transaminase≥3×ULN.
Alanine transaminase or aspartate transaminase≥3×ULN.
Total bilirubin≥1.5×ULN and ≥2×ULN
Alkaline phosphatase≥1.5×ULN and ≥3×ULN.
Alanine transaminase or aspartate transaminase≥3×ULN and total bilirubin≥2×ULN.
Potential Hy's Law cases: alanine transaminase or aspartate transaminase≥3×ULN, total bilirubin≥2×ULN, and alkaline phosphatase ≤2×ULN.

A listing of subjects with any post-baseline abnormal liver function tests is presented.

All clinical laboratory data are listed. Values outside the normal ranges were flagged.

### Vital Signs

For vital signs (blood pressure, pulse, respiratory rate, and body temperature), descriptive statistics and changes from baseline for each scheduled visit are provided. Vital sign results are tabulated based on the result class of normal or abnormal All abnormal values were assessed for clinical significance. Number and percent of subjects within each result class (normal, abnormal but not clinically significant, and abnormal and clinically significant) are presented by time point and treatment group. Additionally, shift tables showing individual subject changes from baseline to each post-baseline time point by treatment group are presented for each vital sign parameter using the following categories: normal, abnormal but not clinically significant, and abnormal and clinically significant. A listing of all vital signs is provided by subject.

Descriptive statistics are provided for 12-lead ECG findings (PR, QRS, QT, and RR.) and changes from baseline for each scheduled visit.

Shift tables are used to summarize individual subject changes from baseline to each post-baseline time point using the following categories: normal, abnormal but not clinically significant, and abnormal and clinically significant. All 12-lead ECG findings are listed by subject

### Study Subjects

### Disposition of Subjects

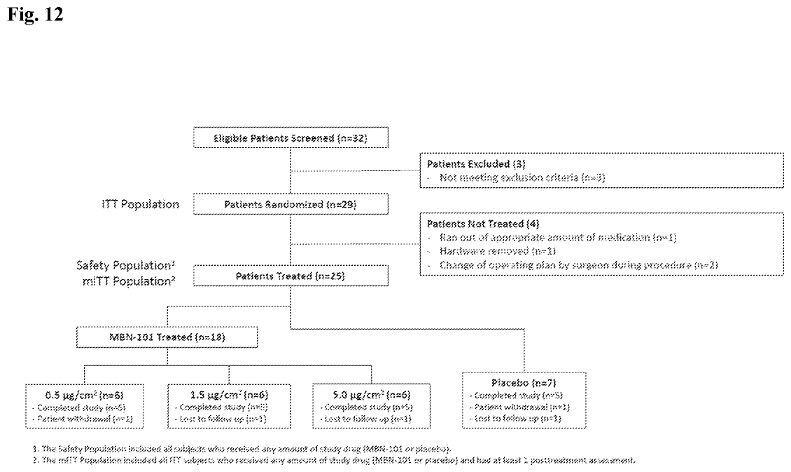

In total, 29 (100%) subjects were randomized (Table 22): 6 subjects in the 0.5 μg/cm² dose group, 9 subjects in the 1.5 μg/cm² dose group, 7 subjects in the 5.0 μg/cm² dose group, and 7 subjects in the placebo group. In total, 25 (86.2%) subjects received study drug (MBN-101 or placebo); 3 subjects in the 1.5 μg/cm² dose group and 1 subject in the 5.0 μg/cm² dose group did not receive study drug (FIG. 12).

TABLE 22
Subject Disposition by Treatment - Intent to Treat Population

| | MBN-101 | MBN-101 | MBN-101 | | |
|---|---|---|---|---|---|
| | 0.025 mg/mL | 0.075 mg/mL | 0.25 mg/mL | | |
| | (0.5 μg/cm [symbol] ) | (1.5 μg/cm [symbol] ) | (5.0 μg/cm [symbol] ) | Placebo | Total |
| | (N = 6) | (N = 9) | (N = 7) | (N = 7) | (N = 29) |
| | n (%) | n (%) | n (%) | n (%) | n (%) |
| Subjects who were randomized | 6 (100 0) | 9 (100.0) | 7 (100.0) | 7 (100.0) | 29 (100.0) |
| Subjects who were treated | 6 (100.0) | 6 (66.7) | 6 (85. [symbol] ) | 7 (100.0) | 25 (8 [symbol] .2) |
| Subjects who were not treated | 0 (0.0) | 3 (33.3) | 1 (14.3) | 0 (0.0) | 4 (1 [symbol] .8) |
| Subjects who completed the study | 5 (83.3) | 5 ( [symbol] .6) | 5 (71.4) | 5 (71. [symbol] ) | 20 (69.0) |
| Subjects who did not complete the study | 1 (16.7) | 4 (44.4) | 2 (28. [symbol] ) | 2 ( [symbol] 8.6) | 9 (31.0) |
| Lost to follow-up | 0 (0.0) | 1 (11.1) | 1 (14.3) | 1 (14.3) | 3 (10. [symbol] ) |
| Withdrawal by subject | 1 (16.7) | 0 (0.0) | 0 (0.0) | 1 (14.3) | 2 ( [symbol] .9) |
| Other | 0 (0.0) | 3 (33.3) | 1 (14.3) | 0 (0.0) | 4 (13.8) |
| Subjects who did not complete the study | 1 (16.7) | 1 ( [symbol] ) | 1 (14.3) | 2 (28. [symbol] ) | 5 (1 [symbol] .2) |
| and were treated | | | | | |
| Subjects who did not complete the study | 0 (0.0) | 3 (33.3) | 1 (14.3) | 0 (0.0) | 4 (13.8) |
| and were not treated | | | | | |

Percentage was calculated using the number of subjects in the column heading as the denominator.
Source: Post-text Table 14.1.1. [symbol] [symbol]
indicates data missing or illegible when filed

### Demographic and Other Baseline Characteristics

The majority of subjects in the study were <65 years of age (21 [84.0%] subjects), white (19 [76.0%] subjects), and male (17 [68.0%] subjects). The mean age of subjects was 48.0 years, the mean weight was 91.38 kg, the mean height was 171.9 cm, and the mean body mass index was 31.13 kg/m² (Table 23).

The majority of subjects (24 [96.0%] subjects) had a fracture injury. In total, 13 (52.0%) subjects had open fracture, high energy injuries: 3 (50.0%) subjects in the 0.5 μg/cm² dose group, 4 (66.7%) subjects in the 1.5 μg/cm² dose group, 4 (66.7%) subjects in the 5.0 μg/cm² dose group, and 2 (28.6%) subjects in the placebo group. In total, 2 (8.0%) subjects had open fracture, low energy injuries: 2 (33.3%) subjects in the 0.5 μg/cm2 dose group. In total, 5 (20.0%) subjects had closed fracture, high energy injuries: 1 (16.7%) subject in the 1.5 μg/cm² dose group, 1 (16.7%) subject in the 5.0 μg/cm² dose group, and 3 (42.9%) subjects in the placebo group. In total, 4 (16.0%) subjects had closed fracture, low energy injuries: 1 (16.7%) subject in the 0.5 μg/cm² dose group, 1 (16.7%) subject in the 1.5 μg/cm² dose group, 1 (16.7%) subject in the 5.0 μg/cm² dose group, and 1 (1 4.3%) subject in the placebo group. In total, 1 (4.0%) subject had an injury classified as other: 1 (14.3%) subject in the placebo group.

In total, 8 (32.0%) subjects had osteomyelitis: 0 (0.0%) subjects in the 0.5 μg/cm² dose group, 3 (50.0%) subjects in the 1.5 μg/cm² dose group, 3 (50.0%) subjects in the 5.0 μg/cm² dose group, and 2 (28.6%) subjects in the placebo group.

The majority of subjects (21 [84.0%] subjects) had lower extremity surgical site locations, and the prevalent fracture fixation methods were plates (16 [64.0%] subjects) and screws (16 [64.0%] subjects). Four (16.0%) subjects had fixation hardware removed prior to baseline, and 8 (32.0%) subjects did not have fixation hardware since nontraumatic osteomyelitis was the underlying illness meeting inclusion criteria.

The mean time from previous surgery to baseline definitive surgery was 65.7 days in the 0.5 μg/cm² dose group, 182.3 days in the 1.5 μg/cm² dose group, 186.3 days in the 5.0 μg/cm² dose group, and 5.3 days in the placebo group.

The majority of subjects (17 [68.0%] subjects) received previous treatment with systemic antibiotics, while 7 (28.0%) subjects received previous treatment with locally or topically administered antibiotics.

In total, the area of osteosynthesis or osteomyelitis site was up to 2.5 cm² in 8 (32.0%) subjects, 25 to 50 cm² in 8 (32.0%) subjects, 50 to 75 cm² in 4 (16.0%) subjects, and 75 to 100 cm², 125 to 150 cm², 150 to 175 cm², 250 to 275 cm², and 300 to 325 cm² in 1 (4.0%) subject each.

Orthopaedic hardware was retained in 6 (24.0%) subjects, while 16 (64.0%) subjects had their hardware either removed and replaced or permanently removed. In total, 7 (28.0%) subjects had new hardware implanted, and 9 (36.0%) subjects did not have new hardware implanted.

The majority of subjects (22 [88.0%] subjects) had wounds closed using sutures, while 7 (28.0%) subjects had staples, 7 (28.0%) subjects had wound vacuum assisted closure, and 4 (1 6.0%) subjects had other methods of wound closure.

TABLE 23
Summary of Demographic and Baseline Characteristics - Safety Population

| | | | | | |
|---|---|---|---|---|---|
| | MBN-101 | MBN-101 | MBN-101 | | |
| | 0.025 mg/mL | 0.075 mg/mL | 0.25 mg/mL | Placebo | Total |
| Demographics/Characteristics | (0.5 μg/cm [symbol] ) | (1.5 μg/cm [symbol] ) | (5.0 μg/cm [symbol] ) | (N = 7) | (N = 25) |
| Category/Statistic | (N = 6) n (%) | (N = 6) n (%) | (N = 6) n (%) | n (%) | n (%) |

Age (years)

| | | | | | |
|---|---|---|---|---|---|
| n | 6 | 6 | 6 | 7 | 25 |
| Mean (SD) | 59.2 (14.55) | 48.2 (17.12) | 37. [symbol] (15.2 [symbol] ) | 47.3 (18.8 [symbol] ) | 48.0 (17. [symbol] ) |

Age group (n, %)

| | | | | | |
|---|---|---|---|---|---|
| <65 years | 4 (66.7) | 5 ( [symbol] .3) | 6 (100.0) | 6 (85.7) | 21 (8 [symbol] 0) |
| ≥65 years | 2 (33.3) | 1 ( [symbol] ) | 0 (0.0) | 1 (14.3) | 4 (16.0) |

Gender (n, %)

| | | | | | |
|---|---|---|---|---|---|
| Male | 4 (66.7) | 3 ( [symbol] 0.0) | 5 (83.3) | 6 ( [symbol] 1.4) | 1 [symbol] (68.0) |
| Female | 2 (33.3) | 3 ( [symbol] 0.0) | 1 (16.7) | 2 (28.6) | [symbol] ( [symbol] 2.0) |

Race (n, %)

| | | | | | |
|---|---|---|---|---|---|
| Asian | 0 (0.0) | 0 (0.0) | 0 (0.0) | 1 (14.3) | [symbol] ( [symbol] .0) |
| Black or African American | 1 (16.7) | 1 (16.7) | 0 (0.0) | 2 (28.6) | 4 (16.0) |
| White | 5 ( [symbol] 3.3) | 4 ( [symbol] 6.7) | 6 (100.0) | 4 ( [symbol] 7.1) | 19 ( [symbol] .0) |
| Other | 0 (0.0) | 1 (16.7) | 0 (0.0) | 0 (0.0) | 1 (4.0) |

Ethnicity (n, %)

| | | | | | |
|---|---|---|---|---|---|
| Hispanic or Latino | 0 (0.0) | 1 (16.7) | 2 (33.3) | 0 (0.0) | [symbol] (12.0) |
| Not Hispanic or Latino | [symbol] (100.0) | 5 (83.3) | 4 (66.7) | 7 (100.0) | 22 (88.0) |

Weight (kg)

| | | | | | |
|---|---|---|---|---|---|
| n | 6 | 6 | 6 | 7 | 25 |
| Mean (SD) | 97.43 (29. [symbol] ) | 80. [symbol] 5 (13.873) | 88.1 [symbol] ( [symbol] 2.884) | 98.69 (27.401) | 91.38 (6.471) |

Height (cm)

| | | | | | |
|---|---|---|---|---|---|
| n | 6 | 6 | 6 | 7 | 25 |
| Mean (SD) | 170.8 (11.81) | 169.7 (6.19) | 171.0 (4.82) | 175.4 (1 [symbol] .00) | 171.9 (9.12) |

BMI (kg/m²)

| | | | | | |
|---|---|---|---|---|---|
| n | 6 | 6 | 6 | 7 | 25 |
| Mean (SD) | 34.07 (13. [symbol] ) | 27.94 (6. [symbol] 8) | 30.09 (1 [symbol] .997) | 32.25 (8.392) | 31.33 (9.724) |

Type of injuries

| | | | | | |
|---|---|---|---|---|---|
| Open fracture, high energy | 3 ( [symbol] 0.0) | 4 (66.7) | 4 (66.7) | 2 (28. [symbol] ) | 1 [symbol] ( [symbol] 2.0) |
| Open fracture, low energy | 2 (33.3) | 0 (0.0) | 0 (0.0) | 0 (0.0) | [symbol] (8.0) |
| Closed fracture, high energy | 0 (0.0) | 1 (16.7) | 1 (16.7) | 3 (42.9) | [symbol] (20.0) |
| Closed fracture, low energy | 1 (16.7) | 1 (16.7) | 1 (16.7) | 1 (14.3) | 4 (16.0) |
| Other | 0 (0.0) | 0 (0.0) | 0 (0.0) | 1 (14.3) | 1 (4.0) |

Presence of osteomyelitis

| | | | | | |
|---|---|---|---|---|---|
| Osteomyelitis | 0 (0.0) | 3 (50.0) | 3 (50.0) | 2 (28.6) | 8 (32.0) |
| Non-osteomyelitis | 6 (100.0) | 3 (50.0) | 3 (50.0) | 5 (71.4) | 17 (68.0) |

Surgical site location

| | | | | | |
|---|---|---|---|---|---|
| Upper extremity | 1 (16.7) | 0 (0.0) | 2 (33.3) | 0 (0.0) | 3 (12.0) |
| Lower extemity | 5 (83.3) | 6 (100.0) | [symbol] (66.7) | 6 (8 [symbol] ) | 21 (84.0) |
| Pelvis | 0 (0.0) | 0 (0.0) | 0 (0.0) | 1 (14.3) | 1 (4.0) |

Method of fracture fixation

| | | | | | |
|---|---|---|---|---|---|
| Pin | 1 (16.7) | 0 (0.0) | 0 (0.0) | 0 (0.0) | 1 (4.0) |
| Plate | 6 (100.0) | [symbol] (50.0) | [symbol] ( [symbol] ) | 4 (57.1) | 16 (64.0) |
| Screw | 6 (100.0) | [symbol] (50.0) | [symbol] ( [symbol] ) | 4 (57.1) | 16 (64.0) |
| Wire | 0 (0.0) | 1 (16.7) | 0 (0.0) | 0 (0.0) | 16 (64.0) |
| External [symbol] | 1 (16.7) | 0 (0.0) | 0 (0.0) | 1 (14.3) | 1 (4.0) |
| None - fixation hardware | 0 (0.0) | 2 (33.3) | 2 (33.3) | 0 (0.0) | 4 (16.0) |
| removed before baseline | | | | | |
| None - osteomyelitis | 0 (0.0) | [symbol] (50.0) | [symbol] (50.0) | 2 (28.6) | 8 (32.0) |

Time from previous surgery to baseline definitive surgery (day)

| | | | | | |
|---|---|---|---|---|---|
| n | 6 | [symbol] | 6 | 6 | 22 |
| Mean (SD) | 65.7 (103.17) | 18 [symbol] .3 (302.09) | 186.3 (234.68) | 5.3 (4.27) | 103.3 (187.06) |

Type of previous treatment

| | | | | | |
|---|---|---|---|---|---|
| Antibiotic administered | 1 (16.7) | 1 (16.7) | [symbol] (50.0) | 2 (28.6) | [symbol] (28.0) |
| locally or topically | | | | | |
| Systemic antibiotics | [symbol] (50.0) | [symbol] (50.0) | 5 (83.3) | 6 (85.7) | 1 [symbol] (68.0) |

Area of the osteosynthesis or osteomyelitis site ( [symbol] )

| | | | | | |
|---|---|---|---|---|---|
| Up to 25 | 2 (33.3) | 1 (16.7) | 2 (33.3) | [symbol] (42.9) | 8 (32.0) |
| 25 to 50 | 2 (33.3) | 2 (33.3) | 2 (33.3) | 2 (28.6) | 8 (32.0) |
| 50 to 75 | 0 (0.0) | 2 (33.3) | 2 (33.3) | 0 (0.0) | 4 (16.0) |
| 75 to 100 | 0 (0.0) | 0 (0.0) | 0 (0.0) | 1 (14.3) | 1 (4.0) |
| 125 to 150 | 1 (16.7) | 0 (0.0) | 0 (0.0) | 0 (0.0) | 1 (4.0) |
| 150 to 175 | 1 (16.7) | 0 (0.0) | 0 (0.0) | 0 (0.0) | 1 (4.0) |
| 250 to 275 | 0 (0.0) | 0 (0.0) | 0 (0.0) | 1 (14.3) | 1 (4.0) |
| 300 to 325 | 0 (0.0) | 1 (16.7) | 0 (0.0) | 0 (0.0) | 1 (4.0) |

Hardware removed

| | | | | | |
|---|---|---|---|---|---|
| Yes | 4 (66.7) | [symbol] (50.0) | 5 (83.3) | 4 ( [symbol] ) | 16 (64.0) |
| No | 2 (33.3) | 2 (33.3) | 0 (0.0) | 2 (28.6) | 6 (24.0) |

New hardware implanted

| | | | | | |
|---|---|---|---|---|---|
| Yes | 4 (66.7) | 1 (16.7) | 0 (0.0) | 2 (28.6) | 7 (28.0) |
| No | 0 (0.0) | 2 (33.3) | 5 (83.3) | 2 (28.6) | 9 (36.0) |

Method of wound closure

| | | | | | |
|---|---|---|---|---|---|
| Sutures | 5 (83.3) | 6 (100.0) | 6 (100.0) | 5 (71.4) | 22 (88.0) |
| Staples | 4 (66.7) | 1 (16.7) | 0 (0.0) | 2 (28.6) | 7 (28.0) |
| Wound vac | 4 (66.7) | 1 (16.7) | 0 (0.0) | 2 (28.6) | 7 (28.0) |
| Other | 1 (16.7) | 1 (16.7) | 1 (16.7) | 1 (14.3) | 4 (16.0) |

Percentage was calculated using the number of subjects in the column heading as the denominator.
Baseline was defined as the last measurement [symbol] assessment prior to the first [symbol] of study [symbol]
Time from previous surgery to baseline [symbol] surgery (day) was calculated [symbol] date of baseline [symbol] surgery [symbol] the date of previous surgery [symbol]
BMI = body mas index [symbol] SD = standard deviation [symbol] vac = vacuum assisted closure [symbol]
Source: Post-text Table 14.1. [symbol] [symbol]
indicates data missing or illegible when filed

### Microbiological Results

### Baseline Pathogen Distribution

Table 24 summarizes an ad-hoc analysis of the distribution of pathogens isolated at baseline from the local microbiologic laboratory for the mITT Population with any baseline pathogens. The total number of subjects with pathogens isolated at baseline (N=17) is less than the total number of subjects in the mITT Population (N=25) since 7 subjects (Subject 001-001, Subject 001-002, Subject 001-003, Subject 001-004, Subject 001-012, Subject 008-001, and Subject 014-010) were cultured at baseline by the local microbiologic laboratory, but did not grow any baseline pathogens, and 1 subject (Subject 014-006) was not cultured at baseline by the local microbiologic laboratory. The most common pathogens isolated at baseline from the local microbiologic laboratory were the following:
S. aureus (unknown MSSA/MRSA)—3 (100.0%) subjects in the 0.5 μg/cm² dose group, 1 (20.0%) subject in the 1.5 μg/cm² dose group, 1 (25.0%) subject in the 5.0 μg/cm² dose group, and 2 (40.0%) subjects in the placebo group
S. aureus (MRSA)—1 (33.3%) subject in the 0.5 μg/cm² dose group, 1 (25.0%)) subject in the 5.0 μg/cm² dose group, and 2 (40.0%) subjects in the placebo group
S. aureus (MSSA)—2 (66.7%) subjects in the 0.5 μg/cm² dose group and 2 (40.0%) subjects in the 1.5 μg/cm² dose group
Enterobacter cloacae—1 (33.3%) subject in the 0.5 μg/cm² dose group, 1 (20.0%)) subject in the 1.5 μg/cm² dose group, 1 (25.0%) subject in the 5.0 μg/cm² dose group, and 1 (20.0%) subject in the placebo group

Table 25 summarizes an ad-hoc analysis of the distribution of pathogens isolated at baseline from the central microbiologic laboratory for the mITT Population with any baseline pathogens. The total number of subjects with pathogens isolated at baseline (N=19) is less than the total number of subjects in the mITT Population (N=25) since 6 subjects (Subject 001-001, Subject 001-003, Subject 001-004, Subject 001-012, Subject 008-001, and Subject 014-010) were cultured at baseline by the central microbiologic laboratory but did not grow any baseline pathogens. The most common pathogens isolated at baseline from the central microbiologic laboratory were the following:
E. cloacae—2 (50.0%) subjects in the 0.5 μg/cm² dose group, 2 (33.3%) subjects in the 1.5 μg/cm² dose group, 1 (25.0%) subject in the 5.0 μg/cm2 dose group, and 1 (20.0%) subject in the placebo group
S. epidermidis—2 (50.0%) subjects in the 0.5 μg/cm² dose group, 2 (33.3%) subjects in the 1.5 μg/cm² dose group, and 1 (20.0%) subject in the placebo group
S. aureus (MRSA)—1 (25.0%,) subject in the 0.5 μg/cm² dose group, 1 (25.0%) subject in the 5.0 μg/cm² dose group, and 2 (40.0%) subjects in the placebo group
S. aureus (MSSA)—1 (25.0%) subject in the 0.5 μg/cm² dose group, 2 (33.3%) subjects in the 1.5 μg/cm² dose group, and 1 (20.0%) subject in the placebo group
Finegoldia magna—1 (25.0%) subject in the 0.5 μg/cm² dose group, 2 (33.3%) subjects in the 1.5 μg/cm² dose group, and 1 (20.0%) subject in the placebo group

TABLE 24
Distribution of Pathogens Isolated at Baseline from Local Microbiologic
Laboratory - mITT Population with any Baseline Pathogens

| | | | | | |
|---|---|---|---|---|---|
| | MBN-101 | MBN-101 | MBN-101 | | |
| | 0.025 mg/mL | 0.075 mg/mL | 0.25 mg/mL | | |
| | (0.5 μg/cm [symbol] ) | (1.5 μg/cm [symbol] ) | (5.0 μg/cm [symbol] ) | Placebo | Total |
| | (N = [symbol] ) | (N = 5) | (N = 4) | (N = [symbol] ) | (N = 17) |
| Baseline Pathogen | n (%) | n (%) | n (%) | n (%) | n (%) |

A [symbol]
Gram-positive
Actinobacteria

| | | | | | |
|---|---|---|---|---|---|
| C [symbol] ynebacterium [symbol] non-speciated | 1 (33.3) | 0 (0.0) | 0 (0.0) | 0 (0.0) | 1 ( [symbol] .9) |

[symbol]

| | | | | | |
|---|---|---|---|---|---|
| Staphhylococcus aureus [symbol] unknown | 3 (100.0) | 1 (20.0) | 1 (25.0) | 2 (40.0) | 7 (41.2) |
| MSSA/MRSA | | | | | |
| Staphhylococcus aureus [symbol] MRSA | 1 (33.3) | 0 (0.0) | 1 (25.0) | 2 (40.0) | 4 (23.5) |
| Staphhylococcus aureus [symbol] MSSA | 2 (66.7) | 2 (40.0) | 0 (0.0) | 0 (0.0) | 4 (23.5) |
| Staphhylococcus [symbol] | 0 (0.0) | 1 (20.0) | 0 (0.0) | 0 (0.0) | 1 (5.9) |
| Bacill [symbol] s [symbol] non-speciated | 1 (33.3) | 0 (0.0) | 0 (0.0) | 0 (0.0) | 1 (5.9) |
| Enterococcus group D | 1 (33.3) | 0 (0.0) | 0 (0.0) | 0 (0.0) | 1 (5.9) |
| [symbol] | 1 (33.3) | 0 (0.0) | 0 (0.0) | 0 (0.0) | 1 (5.9) |

Gram-negative
Enterobac

[symbol]

| | | | | | |
|---|---|---|---|---|---|
| Entero [symbol] | 1 (33.3) | 1 (20.0) | 1 (25.0) | 1 (20.0) | 4 (2 [symbol] ) |
| [symbol] | 1 (33.3) | 1 (20.0) | 1 (25.0) | 0 (0.0) | [symbol] (17.6) |
| Entero [symbol] | 0 (0.0) | 1 (20.0) | 0 (0.0) | 0 (0.0) | 1 (5.9) |
| Esch [symbol] coli | 0 (0.0) | 0 (0.0) | 1 (25.0) | 0 (0.0) | 1 (5.9) |
| [symbol] | 1 (33.3) | 0 (0.0) | 0 (0.0) | 0 (0.0) | 1 (5.9) |

Prot [symbol] bacteria

| | | | | | |
|---|---|---|---|---|---|
| [symbol] | 0 (0.0) | 2 (40.0) | 0 (0.0) | 1 (20.0) | [symbol] (17.6) |
| [symbol] | 0 (0.0) | 0 (0.0) | 1 (25.0) | 0 (0.0) | 1 (5.9) |

A [symbol]
Gram-positive
Actinobacteria

| | | | | | |
|---|---|---|---|---|---|
| [symbol] | 0 (0.0) | 0 (0.0) | 0 (0.0) | 1 (20.0) | 1 (5.9) |

[symbol]

| | | | | | |
|---|---|---|---|---|---|
| [symbol] | 0 (0.0) | 2 (40.0) | 0 (0.0) | 0 (0.0) | 2 ( [symbol] ) |
| [symbol] from broth only | 0 (0.0) | 0 (0.0) | 0 (0.0) | 1 (20.0) | 1 (5.9) |

Both [symbol] and [symbol]
Yeast
A [symbol] ota

| | | | | | |
|---|---|---|---|---|---|
| [symbol] | 0 (0.0) | 0 (0.0) | 1 (25.0) | 0 (0.0) | 1 (5.9) |

Percentage was calculated using the number of subjects in the column heading as the denominator.
Note:
Sorted by respi [symbol] type [symbol] Gram-staining and descending frequency [symbol]
MRSA = [symbol] resistent Staphhylococcus aureus [symbol] MSSA = [symbol] sensitive Staphhylococcus aureus [symbol]
Source: [symbol] Table [symbol] [symbol]
indicates data missing or illegible when filed

TABLE 25
Distribution of Pathogens Isolated at Baseline from Central Microbiologic
Laboratory - mITT Population with any Baseline Pathogens

| | | | | | |
|---|---|---|---|---|---|
| | MBN-101 | MBN-101 | MBN-101 | | |
| | 0.025 mg/mL | 0.075 mg/mL | 0.25 mg/mL | | |
| | (0.5 μg/cm [symbol] ) | (1.5 μg/cm [symbol] ) | (5.0 μg/cm [symbol] ) | Placebo | Total |
| | (N = 4) | (N = 6) | (N = 4) | (N = [symbol] ) | (N = 19) |
| Baseline Pathogen | n (%) | n (%) | n (%) | n (%) | n (%) |

A [symbol]
Gram-positive
Actinobacteria

| | | | | | |
|---|---|---|---|---|---|
| [symbol] bacterium [symbol] | 1 (25.0) | 0 (0.0) | 0 (0.0) | 1 (20.0) | 2 ( [symbol] .5) |
| [symbol] bacterium [symbol] non-speciated | 1 (25.0) | 1 (16.7) | 0 (0.0) | 0 (0.0) | 2 ( [symbol] .5) |
| A [symbol] | 0 (0.0) | 1 (16.7) | 0 (0.0) | 0 (0.0) | 1 (5.3) |
| [symbol] bacterium [symbol] | 0 (0.0) | 1 (16.7) | 0 (0.0) | 0 (0.0) | 1 (5.3) |
| [symbol] bacterium [symbol] | 1 (25.0) | 0 (0.0) | 0 (0.0) | 0 (0.0) | 1 (5.3) |
| [symbol] bacterium [symbol] | 0 (0.0) | 1 (16.7) | 0 (0.0) | 0 (0.0) | 1 (5.3) |
| D [symbol] | 0 (0.0) | 1 (16.7) | 0 (0.0) | 0 (0.0) | 1 (5.3) |

Fi

[symbol]

| | | | | | |
|---|---|---|---|---|---|
| St [symbol] | 2 (50.0) | 2 (33.3) | 0 (0.0) | 1 (20.0) | [symbol] ( [symbol] ) |
| St aureus MRSA | 1 (25.0) | 0 (0.0) | 1 (25.0) | 2 (40.0) | 4 (21.1) |
| St aureus MSSA | 1 (25.0) | 2 (33.3) | 0 (0.0) | 1 (20.0) | 4 (21.1) |
| St [symbol] | 2 (50.0) | 0 (0.0) | 0 (0.0) | 1 (20.0) | [symbol] (15.8) |
| Entero [symbol] | 1 (25.0) | 0 (0.0) | 0 (0.0) | 0 (0.0) | 1 (5.3) |
| [symbol] non-speciated | 0 (0.0) | 1 (16.7) | 0 (0.0) | 0 (0.0) | 1 (5.3) |
| St [symbol] | 0 (0.0) | 0 (0.0) | 0 (0.0) | 1 (20.0) | 1 (5.3) |
| Sta [symbol] | 0 (0.0) | 0 (0.0) | 0 (0.0) | 1 (20.0) | 1 (5.3) |
| St [symbol] | 0 (0.0) | 0 (0.0) | 1 (25.0) | 0 (0.0) | 1 (5.3) |
| St [symbol] | 1 (25.0) | 0 (0.0) | 0 (0.0) | 0 (0.0) | 1 (5.3) |
| St [symbol] | 1 (25.0) | 0 (0.0) | 0 (0.0) | 0 (0.0) | 1 (5.3) |
| St [symbol] | 1 (25.0) | 0 (0.0) | 0 (0.0) | 0 (0.0) | 1 (5.3) |

Gram-negative
Enterobac

[symbol]

| | | | | | |
|---|---|---|---|---|---|
| Entero [symbol] | 2 (50.0) | 2 (33.3) | 1 (25.0) | 1 (20.0) | [symbol] (31.6) |
| S [symbol] | 1 (25.0) | 1 (16.7) | 1 (25.0) | 0 (0.0) | [symbol] (15.8) |
| Esch [symbol] coli | 0 (0.0) | 0 (0.0) | 1 (25.0) | 0 (0.0) | 1 (5.3) |
| K [symbol] | 1 (25.0) | 0 (0.0) | 0 (0.0) | 0 (0.0) | 1 (5.3) |

Prot [symbol] bacteria

| | | | | | |
|---|---|---|---|---|---|
| Pseudomonas aer [symbol] ginosa | 0 (0.0) | 2 (33.3) | 0 (0.0) | 1 (20.0) | [symbol] (15.8) |
| [symbol] | 0 (0.0) | 0 (0.0) | 0 (0.0) | 1 (20.0) | 1 (5.3) |

A [symbol]
Gram-positive
Actinobacteria

| | | | | | |
|---|---|---|---|---|---|
| [symbol] | 1 (25.0) | 0 (0.0) | 2 (50.0) | 0 (0.0) | [symbol] (15.8) |

F

[symbol]

| | | | | | |
|---|---|---|---|---|---|
| [symbol] | 1 (25.0) | 2 (33.3) | 0 (0.0) | 1 (20.0) | 4 (21.1) |
| [symbol] | 0 (0.0) | 1 (16.7) | 0 (0.0) | 0 (0.0) | 1 (5.3) |
| [symbol] | 0 (0.0) | 1 (16.7) | 0 (0.0) | 0 (0.0) | 1 (5.3) |

Percentage was calculated using the number of subjects in the column heading as the denominator.
Note:
Sorted by respi [symbol] type [symbol] Gram-staining and descending frequency [symbol]
MRSA = [symbol] resistent Staphhylococcus aureus [symbol] MSSA = [symbol] sensitive Staphhylococcus aureus [symbol]
Source: [symbol] Table [symbol] [symbol]
indicates data missing or illegible when filed

Table 26 and Table 27 summarize an ad-hoc analysis of baseline microbiological results from the central laboratory of Gram-positive aerobes for the mITT Population with any baseline pathogen isolates with resistance to 1 or more antibiotics. Table 26 and Table 27 alphabetically list the A to Land M to Z tested antibiotics, respectively.

Bismuth-1,2-ethanedithiol (BisEDT, the active pharmaceutical ingredient in MBN-101) demonstrated low MICs against Gram-positive aerobic baseline pathogens, including those with multidrug antibiotic resistance. The BisEDT mean MIC of Gram-positive aerobes was the following:
Actinomyces species (spp.)—0.25 mg/L (Actinomyces turicensis)
Corynebacterium spp.—0.03 mg/L (Corynebacterium jeikeium), 0.06 mg/L (Corynebacterium resistens, Corynebacterium simulans, and non-speciated Corynebacterium), and 0.12 mg/L (Corynebacterium amycolatum)
Dermabacter spp.—4.00 mg/L (Dermabacter hominis)
Enterococcus spp.—2.00 mg/L (Enterococcus faecalis)
Staphylococcus spp.—0.02 mg/L (S. epidermidis), 0.03 mg/L (Staphylococcus lugdunensis), 0.06 mg/L (Staphylococcus hominis, Staphylococcus pasteuri, and Staphylococcus warneri), 0.10 mg/L (S. aureus [MRSA]), 0.12 mg/L S. aureus [MSSA] and Staphylococcus haemolyticus), 0.25 mg/L (Staphylococcus arlettae)
Streptococcus spp.—0.50 mg/L (Streptococcus oralis)

Gram-positive aerobes demonstrated resistance to the following antibiotics:
Ceftriaxone—C. jeikeium (2 [100.0%] subjects) and C. resistens (1 [100.0%] subject)
Clindamycin—C. amycolatum (1 [100.0%] subject), C. jeikeium (2 [100.0%] subjects), non-speciated Corynebacterium (1 [50.0%] subject), S. aureus [MRSA) (1 [25.0%] subject), S. epidermidis (1 [20.0%] subject), S. haemolyticus (1 [100.0%] subject), and S. pasteuri (1 [100.0%] subject)
Erythromycin—C. amycolatum (1 [100.0%] subject), C. jeikeium (1 [50.0%] subject), non-speciated Corynebacterium (1 [50.0%] subject), D. hominis (1 [100.0%] subject), and S. oralis (1 [100.0%] subject)
Levofloxacin—S. aureus (MRSA) (4 [100.0%] subjects), S. epidermidis (2 [40.0%] subjects), S. haemolyticus (1 [100.0%] subject), and S. hominis (1 [100.0%] subject)
Metronidazole—A. turicensis (1 [100.0%] subject)
Oxacillin—S. arlettae (1 [100.0%1] subject), S. aureus (MRSA) (4 [100.0%] subjects), S. epidermidis (2 [40.0%] subjects), S. haemolyticus (1 [100.0%] subject), S. hominis (1 [100.0%] subject), and S. lugdunensis (2 [66.7%] subjects)
Penicillin G—C. jeikeium (2 [100.0%] subjects), C. resistens (1 [100.0%] 1 subject), and non-speciated Corynebacterium (1 [50.0%] subject)
Trimethoprim/Sulfamethoxazole—non-speciated Corynebacterium (1 [50.0%] subject), D. hominis (1 [100.0%] subject), S. arlettae (1 [100.0%] subject), S. aureus (MRSA) (1 [25.0%] subject), S. epidermidis (3 [60.0%] subjects), and S. hominis (1 [100.0%] subject)

TABLE 26
Summary of Baseline Microbiological Results from Central Laboratory - Gram-positive Aerobes - Modified Intent-to-Treat
Population With Any Baseline Pathogen (Isolates with Resistance to One or More Antibiotics [A-L Antibiotics])

| | BisEDT | | | | | | | | |
|---|---|---|---|---|---|---|---|---|---|
| | Mean | | | | | | | | |
| Taxonomy | MIC mg/L | Mean MIC mg/L n (% resistant) | | | | | | | |
| Species | (±SD) | AMC | AMP | CFZ | CLI | CRO | ERY | LVX | LZD |

Actinobacteria (High-GC)

| | | | | | | | | | |
|---|---|---|---|---|---|---|---|---|---|
| Actinomyces turicensis (N′ = 1) | 0.25 | — | — | — | 0.25 | — | — | — | — |
| | (NA) | | | | 0 (0.0) | | | | |
| Corynebacterium amycolatum (N′ = 1) | 0.12 | 1.00 | — | — | 2.00 | 1.00 | 4.00 | 8.00 | 0.12 |
| | (NA) | 0 (0.0) | | | 1 (100.0) | 0 (0.0) | 1 (100.0) | 0 (0.0) | 0 (0.0) |
| Corynebacterium jeikeium (N′ = 2) | 0.03 | 3.00 | — | — | 2.00 | 4.00 | 2.25 | 0.18 | 0.38 |
| | (0.000) | 0 (0.0) | | | 2 (100.0) | 2 (100.0) | 1 (50.0) | 0 (0.0) | 0 (0.0) |
| Corynebacterium resistens (N′ = 1) | 0.06 | 8.00 | — | — | 0.50 | 4.00 | 0.02 | 0.12 | 0.25 |
| | NA | 0 (0.0) | | | 0 (0.0) | 1 (100.0) | 0 (0.0) | 0 (0.0) | 0 (0.0) |
| Corynebacterium non-speciated (N′ = 2) | 0.06 | 4.03 | — | — | 1.06 | 0.75 | 2.02 | 0.18 | 0.18 |
| | (0.000) | 0 (0.0) | | | 1 (50.0) | 0 (0.0) | 1 (50.0) | 0 (0.0) | 0 (0.0) |
| Dermabacter hominis (N′ = 1) | 4.00 | 0.50 | — | — | 0.50 | 0.50 | 2.00 | 1.00 | 0.50 |
| | (NA) | 0 (0.0) | | | 0 (0.0) | 0 (0.0) | 1 (100.0) | 0 (0.0) | 0 (0.0) |

Firmicutes (Low-GC)

| | | | | | | | | | |
|---|---|---|---|---|---|---|---|---|---|
| Staphylococcus arlettae (N′ = 1) | 0.25 | 0.25 | 0.50 | 2.00 | 0.25 | — | — | 0.50 | 2.00 |
| | (NA) | 0 (0.0) | 0 (0.0) | 0 (0.0) | 0 (0.0) | | | 0 (0.0) | 0 (0.0) |
| Staphylococcus aureus, MRSA (N′ = 4) | 0.10 | 8.00 | 28.00 | 13.00 | 4.16 | — | — | 8.00 | 1.25 |
| | (0.101) | 0 (0.0) | 0 (0.0) | 0 (0.0) | 1 (25.0) | | | 4 (100.0) | 0 (0.0) |
| Staphylococcus epidermidis (N′ = 5) | 0.02 | 1.05 | 2.60 | 1.87 | 3.66 | — | — | 2.52 | 0.80 |
| | (0.008) | 0 (0.0) | 0 (0.0) | 0 (0.0) | 1 (20.0) | | | 2 (40.0) | 0 (0.0) |
| Staphylococcus haemolyticus (N′ = 1) | 0.12 | 2.00 | 16.00 | 4.00 | 8.00 | — | — | 8.00 | 1.00 |
| | (NA) | 0 (0.0) | 0 (0.0) | 0 (0.0) | 1 (100.0) | | | 1 (100.0) | 0 (0.0) |
| Staphylococcus hominis (N′ = 1) | 0.06 | 2.00 | 4.00 | 8.00 | 0.12 | — | — | 8.00 | 0.50 |
| | (NA) | 0 (0.0) | 0 (0.0) | 0 (0.0) | 0 (0.0) | | | 1 (100.0) | 0 (0.0) |
| Staphylococcus lugdunensis (N′ = 3) | 0.03 | 3.67 | 7.33 | 6.83 | 0.03 | — | — | 0.92 | 0.83 |
| | (0.023) | 0 (0.0) | 0 (0.0) | 0 (0.0) | 0 (0.0) | | | 0 (0.0) | 0 (0.0) |

Firmicutes (Low-GC) (continued)

| | | | | | | | | | |
|---|---|---|---|---|---|---|---|---|---|
| Staphylococcus pasteuri (N′ = 1) | 0.06 | 0.12 | 0.25 | 0.25 | 1.00 | — | — | 0.25 | 1.00 |
| | (NA) | 0 (0.0) | 0 (0.0) | 0 (0.0) | 1 (100.0) | | | 0 (0.0) | 0 (0.0) |
| Streptococcus oralis (N′ = 1) | 0.50 | 0.06 | — | — | 0.03 | 0.06 | 1.00 | 2.00 | 1.00 |
| | (NA) | 0 (0.0) | | | 0 (0.0) | 0 (0.0) | 1 (100.0) | 0 (0.0) | 0 (0.0) |

Percentage was calculated using N′ as the denominator, where N′ was the number of unique organisms tested for the specified antimicrobial.
Note:
If the same organism was isolated from multiple samples within the baseline visit, the highest MIC to the specified antimicrobial was used.
AMC = amoxicilin clavulanate; AMP = ampicilin; BisEDT = Bismuth-1,2-ethanedithiol; CFZ = cefazolin; CLI = clindamycin; CRO = ceftriaxone; ERY = erythromycin; GC = guanine-cytosine; LVX = levofloxacin; LZD = linezolid; MIC = minimum inhibitory concentration; MRSA = methicillin-resistant Staphylococcus aureus; NA = not applicable; SD = standard deviation.
Source: Post-text Ad-hoc Table 14.1.3a3

TABLE 27
Summary of Baseline Microbiological Results from Central Laboratory - Gram-positive Aerobes - Modified Intent-
to-Treat Population With Any Baseline Pathogen (Isolates with Resistance to One or More Antibiotics [M-Z Antibiotics])

| | BisEDT | | | | | | | | | |
|---|---|---|---|---|---|---|---|---|---|---|
| | Mean | | | | | | | | | |
| Taxonomy | MIC mg/L | Mean MIC mg/L n (% resistant) | | | | | | | | |
| Species | (±SD) | MEM | MTZ | MIN | MXF | OXA | PEN | SXT | TZP | VAN |

Actinobacteria (High-GC)

| | | | | | | | | | | |
|---|---|---|---|---|---|---|---|---|---|---|
| Actinomyces turicensis (N′ = 1) | 0.25 | 0.25 | 16.00 | — | 2.00 | — | — | — | 1.00 | — |
| | (NA) | 0 (0.0) | 1 (100.0) | | 0 (0.0) | | | | 0 (0.0) | |
| Corynebacterium amycolatum (N′ = 1) | 0.12 | — | — | — | — | — | 1.00 | 0.25 | 1.00 | 0 25 |
| | (NA) | | | | | | 0 (0.0) | 0 (0.0) | 0 (0.0) | 0 (0.0) |
| Corynebacterium jeikeium (N′ = 2) | 0.03 | — | — | — | — | — | 4.00 | 2.00 | 1.00 | 0 38 |
| | (0.000) | | | | | | 2 (100.0) | 0 (0.0) | 0 (0.0) | 0 (0.0) |
| Corynebacterium resistens (N′ = 1) | 0.06 | — | — | — | — | — | 4.00 | 2.00 | 1.00 | 1.00 |
| | (NA) | | | | | | 1 (100.0) | 0 (0.0) | 0 (0.0) | 0 (0.0) |
| Corynebacterium non-speciated (N′ = 2) | 0.06 | — | — | — | — | — | 2.06 | 2.25 | 1.00 | 0.12 |
| | (0.000) | | | | | | (50.0) | 1 (30.0) | 0 (0.0) | 0 (0.0) |
| Dermabacter hominis (N′ = 1) | 4.00 | — | — | — | — | — | 0.50 | 4.00 | 0.50 | 0.25 |
| | (NA) | | | | | | 0 (0.0) | 1 (1.00.0) | 0 (0.0) | 0 (0.0) |

Firmicutes (Low-GC)

| | | | | | | | | | | |
|---|---|---|---|---|---|---|---|---|---|---|
| Staphylococcus arlettae (N′ = 1) | 0.25 | — | — | 0.25 | — | 2.00 | — | 8.00 | — | 2.00 |
| | (NA) | | | 0 (0.0) | | 1 (100.0) | | 1 (100.0) | | 0 (0.0) |
| Staphylococcus aureus, MRSA (N′ = 4) | 0.10 | — | — | 0.11 | — | 4 00 | — | 2.09 | — | 1.25 |
| | (0.101) | | | 0 (0.0) | | 4 (100.0) | | 1 (25.0) | | 0 (0.0) |
| Staphylococcus epidermidis (N′ = 5) | 0.02 | — | — | 0.18 | — | 125 | — | 4.42 | — | 150 |
| | (0.008) | | | 0 (0.0) | | 2 (40.0) | | 3 (60.0) | | 0 (0.0) |
| Staphylococcus haemolyticus (N′ = 1) | 0.12 | — | — | 0.12 | — | 4.00 | — | 0.25 | — | 1.00 |
| | (NA) | | | 0 (0.0) | | 1 (100.0) | | 10.0 | | () (0.0) |
| Staphylococcus hominis (N′ = 1) | 0.06 | — | — | 0.12 | — | 4.00 | — | 8.00 | — | 2.00 |
| | (NA) | | | 0 (0.0) | | 1 (100.0) | | I (100.0) | | 0 (0.0) |

Firmicutes (Low-GC) (continued)

| | | | | | | | | | | |
|---|---|---|---|---|---|---|---|---|---|---|
| Staphylococcus lugdunensis (N′ = 3) | 0.03 | — | — | 0.05 | — | 2.83 | — | 0.16 | — | 0.83 |
| | (0 023) | | | 0 (0 0) | | 2. (66.7) | | 0 (0.0) | | 0 (0.0) |
| Staphylococcus pasteuri (N′ = 1) | 0.06 | — | — | 0.06 | — | 0.25 | — | 0.12 | — | 1.00 |
| | (NA) | | | 0 (0.0) | | 0 (0.0) | | 0: (0.0) | | 0 (0.0) |
| Streptococcus oralis (N′ = 1) | 0.30 | — | — | — | — | — | 0.06 | 0.12 | 0.06 | 0.50 |
| | (NA) | | | | | | 0 (0.0) | 0 (0.0) | 0 (0.0) | 0 (0.0) |

Percentage was calculated using N′ as the denominator, where N′ was the number of unique organisms tested for the specified antimicrobial.
Note:
If the same organism was isolated from multiple samples within the baseline visit, the highest MIC to the specified antimicrobial was used.
BisEDT = Bismuth-1,2-ethanedithiol; GC = guanine-cytosine; MEM = meropenem; MIC = minimum inhibitory concentration; MIN = minocycline; MRSA = methicillin-resistant Staphylococcus aureus; MTZ = metronidazole; MXF = moxifloxacin; NA = not applicable; OXA = oxacillin, PEN = penicillin G; SD = standard deviation; SXT = trimethoprim/sulfamethoxazole: TZP = piperacillin/tazobactam; VAN = vancomycin.
Source: Post-text Ad-hoc Table 14.1.3a3

Table 28 summarizes an ad-hoc analysis of baseline microbiological results from the central laboratory of anaerobes for the mITT Population with any baseline pathogen isolates with resistance to 1 or more antibiotics.

Bismuth-1,2-ethanedithiol (BisEDT, the active pharmaceutical ingredient in MBN-101) demonstrated low MICs against anaerobic baseline pathogens, including those with multidrug antibiotic resistance. The BisEDT mean MIC of anaerobes was the following:
Anaerococcus spp.—0.06 mg/L (Anaerococcus murdochii) and 0.25 mg/L (non-speciated Anaerococcus)
Clostridium spp.—0.25 mg/L (Clostridium sphenoides)
Cutibacterium (Propionibacterium) spp.—0.06 mg/L (Cutibacterium [Propionibacterium] acnes)
Finegoldia spp.—0.22 mg/L (F. magna)
Peptoniphilus spp.—0.06 mg/L (Peptoniphilus gorbachii)
Prevotella spp.—0.06 mg/L (Prevotella bergensis)
   Gram-positive anaerobes demonstrated resistance to the following antibiotics:
Clindamycin—A. murdochii (1 [100.0%] subject), C. sphenoides (1 [100.0%] subject), F. magna (2 [50.0%] subjects), and P. gorbachii (1 [100.0%] subject)
Metronidazole—C. (Propionibacterium) acnes (3 [100.0%] subjects) and non-speciated Anaerococcus (1 [100.0%] subject)
Moxifloxacin—F. magna (1 [25.0%] subject)
   No Gram-negative anaerobes demonstrated resistance to antibiotics.

TABLE 28
Summary of Baseline Microbiological Results from Central Laboratory - Anaerobes - Modified Intent-to-
Treat Population With Any Baseline Pathogen (Isolates with Resistance to One or More Antibiotics)

| | BisEDT | | | | | |
|---|---|---|---|---|---|---|
| Taxonomy | MIC mg/L | Mcan MIC mg/L n (% resistant) | | | | |
| Species | (±SD) | CLI | MEM | MTZ | MXF | TZP |

Actinobacteria (High-GC nam-positive)

| | | | | | | |
|---|---|---|---|---|---|---|
| Cutibacterium (Proprionibacterium) acnes (N′ = 3) | 0.06 | 0.25 | 0.06 | 16.00 | 0.25 | 0.29 |
| | (0.000) | 0 (0.0) | 0 (0.0) | 3 (100.0) | 0 (0.0) | 0 (0.0) |

Firmicutes (Low-GC Gram-positive)

| | | | | | | |
|---|---|---|---|---|---|---|
| Anaerococcus murdochii (N′ = 1) | 0.06 | 8.00 | 0.06 | 1.00 | 4.00 | 0.50 |
| | (NA) | 1 (100.0) | 0 (0.0) | 0 (0.0) | 0 (0.0) | 0 (0.0) |
| Anaerococcus non-speciated (N′ = 1) | 0.25 | 0.25 | 0.25 | 16.00 | 2.00 | 1 00 |
| | (NA) | 0 (0.0) | 0 (0.0) | 1 (100.0) | 0 (0.0) | 0 (0.0) |
| Clostridium sphenoides (N′ = 1) | 0.25 | 8.00 | 2.00 | 0.32 | 4.00 | 16.00 |
| | (NA) | 1 (100.0) | 0 (0.0) | 0 (0.0) | 0 (0.0) | 0 (0.0) |
| Finegoldia magna (N′ = 4) | 0.22 | 4.38 | 0.06 | 0.56 | 4.16 | 0.34 |
| | (0.065) | 2 (50.0) | 0 (0.0) | 0 (0.0) | 1 (25.0) | 0 (0.0) |
| Peptoniphilus gorbachii (N′ = 1) | 0.06 | 8.00 | 0.06 | 0.50 | 0.50 | 0.03 |
| | (NA) | 1 (100.0) | 0 (0.0) | 0 (0.0) | 0 (0.0) | 0 (0.0) |

Percentage was calculated using N′ as the denominator, where N′ was the number of unique organisms tested for the specified antimicrobial.
Note:
If the same organism was isolated from multiple samples within the baseline visit, the highest MIC to the specified antimicrobial was used.
BisEDT = Bismuth-1,2-ethanedithiol; CLI = clindamycin; GC = guanine-cytosine; MEM = meropenem; MIC = minimum inhibitory concentration; MTZ = metronidazole; NA = not applicable; SD = standard deviation; TZP = piperacillin/lazobactum.
Source: Post-text Ad-hoc Table 14.1.3a3

### Medical History

None of the subjects in the mITT population had a history of connective tissue disease/autoimmune disease. A history of HIV/AIDS was reported for 1 (16.7%) subject in the 0.5 μg/cm² dose group. A history of renal disease was reported for 1 (16.7%) subject in the 0.5 μg/cm² dose group, 1 (16.7%) subject in the 1.5 μg/cm² dose group, and 2 (28.6%) subjects in the placebo group. A history of diabetes mellitus was reported for 1 (16.7%) subject in the 0.5 μg/cm² dose group, 1 (16.7%) subject in the 1.5 μg/cm² dose group, and 1 (14.3%) subject in the placebo group.

### Concomitant Systemic Antibiotic Treatment

Table 29 summarizes the concomitant systemic antibiotics for the Safety Population. All 25 (100.0%) subjects took at least 1 concomitant systemic antibiotic to treat the infected bone site during the study. The most common concomitant systemic antibiotic used to treat the infected bone site was intravenous vancomycin (4 [66.7%] subjects in the 0.5 μg/cm² dose group, 3 [50.0%] subjects in the 1.5 μg/cm² dose group, 3 [50.0%] subjects in the 5.0 μg/cm² dose group, and 5 [71.4%] subjects in the placebo group). Prior and/or concomitant topical antibiotics (i.e., within 30 days prior to informed consent until study participation was complete) were administered at the infected bone site in 8 subjects.

TABLE 29
Summary of Concomitant Systemic Antibiotics Used to Treat Infected Bone Site - Safety Population

| | MBN-101 | MBN-101 | MBN-101 | | |
|---|---|---|---|---|---|
| | 0.025 mg/mL | 0.075 mg/mL | 0.25 mg/mL | | |
| | (0.5 μg/cm [symbol] ) | (1.5 μg/cm [symbol] ) | (5.0 μg/cm [symbol] ) | Placebo | Total |
| | (N = 6) | (N = 6) | (N = 6) | (N = 7) | (N = 25) |
| ATC Classification Preferred Term | n (%) | n (%) | n (%) | n (%) | n (%) |
| Any concomitant systemic antibiotics | 6 (100.0) | 6 (100.0) | 6 (100.0) | 7 (100.0) | 25 (100.0) |
| used to treat infected bone site | | | | | |
| Glycopeptide antibacterials | 4 (66.7) | 3 (50.0) | 3 (50.0) | 6 (85.7) | 16 (64.0) |
| Vancomycin [1] | 4 (66.7) | 3 (50.0) | 3 (50.0) | 5 (71.4) | 15 (60.0) |
| Vancomycin hydrochloride [1] | 0 (0.0) | 0 (0.0) | 0 (0.0) | 1 (14.3) | 1 (4.0) |
| First-generation cephalosporins | 3 (50.0) | 6 (100.0) | 4 (66.7) | 2 (28.6) | 15 (60.0) |
| Cefazolin | 3 (50.0) | 5 (83.3) | 4 (66.7) | 2 (28.6) | 14 (56.0) |
| Cefadroxil | 1 (16.7) | 0 (0.0) | 0 (0.0) | 0 (0.0) | 1 (4.0) |
| Cefalexin | 1 (16.7) | 0 (0.0) | 0 (0.0) | 0 (0.0) | 1 (4.0) |
| Cefazolin sodium | 0 (0.0) | 1 (16.7) | 0 (0.0) | 0 (0.0) | 1 (4.0) |
| Combinations of penicillins [symbol] including | 3 (50.0) | 1 (16.7) | 1 (16.7) | 3 (42.9) | 8 (32.0) |
| beta lactamase inhibitors | | | | | |
| PIP/TAZO | 3 (50.0) | 0 (0.0) | 1 (16.7) | 3 (42.9) | 7 (28.0) |
| Spek [symbol] | 1 (16.7) | 1 (16.7) | 0 (0.0) | 0 (0.0) | 2 (8.0) |
| Fluoro [symbol] olones | 2 (33.3) | 2 (33.3) | 2 (33.3) | 2 (28.6) | 8 (32.0) |
| Ciprofloxacin | 1 (16.7) | 2 (33.3) | 0 (0.0) | 1 (14.3) | 4 (16.0) |
| Levofloxacin | 1 (16.7) | 0 (0.0) | 2 (33.3) | 1 (14.3) | 4 (16.0) |
| Fourth-generations cep [symbol] losporins | 1 (16.7) | 4 (66.7) | 1 (16.7) | 1 (14.3) | 7 (28.0) |
| Cefepi [symbol] e | 1 (16.7) | 4 (66.7) | 1 (16.7) | 0 (0.0) | 6 (24.0) |
| Cefepi [symbol] e hydroc [symbol] oride | 0 (0.0) | 0 (0.0) | 0 (0.0) | 1 (14.3) | 1 (4.0) |
| Antibiotics | 1 (16.7) | 0 (0.0) | 1 (16.7) | 3 (42.9) | 5 (20.0) |
| Rifampicin | 1 (16.7) | 0 (0.0) | 1 (16.7) | 1 (14.3) | 3 (12.0) |
| Vancomycin [2] | 0 (0.0) | 0 (0.0) | 0 (0.0) | 1 (14.3) | 1 (4.0) |
| Vancomycin hydrochloride [2] | 0 (0.0) | 0 (0.0) | 0 (0.0) | 1 (14.3) | 1 (4.0) |
| Combinations of sulfonamides and | 1 (16.7) | 1 (16.7) | 1 (16.7) | 1 (14.3) | 4 (16.0) |
| trimethoprim, including derivatives | | | | | |
| Bactrim | 1 (16.7) | 1 (16.7) | 1 (16.7) | 1 (14.3) | 4 (16.0) |
| Tetracyclines | 1 (16.7) | 2 (33.3) | 1 (16.7) | 0 (0.0) | 4 (16.0) |
| Doxycycline | 0 (0.0) | 1 (16.7) | 1 (16.7) | 0 (0.0) | 2 (8.0) |
| Doxycycline monohydrate | 1 (16.7) | 1 (16.7) | 0 (0.0) | 0 (0.0) | 2 (8.0) |
| Doxycycline [symbol] clate | 1 (16.7) | 0 (0.0) | 0 (0.0) | 0 (0.0) | 1 (4.0) |
| Imidazole derivatives | 1 (16.7) | 0 (0.0) | 0 (0 0) | 1 (14.3) | 2 (8.0) |
| Metronidazole | 1 (16.7) | 0 (0.0) | 0 (0.0) | 1 (14.3) | 2 (8.0) |
| Lincosamides | 1 (16.7) | 0 (0.0) | 1 (16.7) | 0 (0.0) | 2 (8.0) |
| Clindamycin | 1 (16.7) | 0 (0.0) | 1 (16.7) | 0 (0.0) | 2 (8.0) |
| Other antibacterials | 1 (16.7) | 0 (0.0) | 1 (16.7) | 0 (0.0) | 2 (8.0) |
| Daptomycin | 0 (0.0) | 0 (0.0) | 1 (16.7) | 0 (0.0) | 1 (4.0) |
| Linezolid | 1 (16.7) | 0 (0.0) | 0 (0.0) | 0 (0.0) | 1 (4.0) |
| Beta lactamase resistant penicillins | 0 (0.0) | 1 (16.7) | 0 (0.0) | 0 (0.0) | 1 (4.0) |
| Nafcillin | 0 (0.0) | 1 (16.7) | 0 (0.0) | 0 (0.0) | 1 (4.0) |
| Monobac [symbol] | 1 (16.7) | 0 (0.0) | 0 (0.0) | 0 (0.0) | 1 (4.0) |
| A [symbol] | 1 (16.7) | 0 (0.0) | 0 (0.0) | 0 (0.0) | 1 (4.0) |
| Penicillins with extended specim [symbol] n | 0 (0.0) | 0 (0.0) | 1 (16.7) | 0 (0.0) | 1 (4.0) |
| Amoxicillin | 0 (0.0) | 0 (0.0) | 1 (16.7) | 0 (0.0) | 1 (4.0) |
| Third-generation cephalosporins | 1 (16.7) | 0 (0.0) | 0 (0.0) | 0 (0.0) | 1 (4.0) |
| Cefpodoxi [symbol] e | 1 (16.7) | 0 (0.0) | 0 (0.0) | 0 (0.0) | 1 (4.0) |
| Ceftr [symbol] xone | 1 (16.7) | 0 (0.0) | 0 (0.0) | 0 (0.0) | 1 (4.0) |
| Triazole derivatives | 0 (0.0) | 0 (0.0) | 0 (0.0) | 1 (14.3) | 1 (4.0) |
| Vorico [symbol] zole | 0 (0.0) | 0 (0.0) | 0 (0.0) | 1 (14.3) | 1 (4.0) |

Coding was based on the WHO Drug Dictionary (March 2015) [symbol]
Percentage was calculated using the number of subjects in the column heading as the denominator [symbol]
Concomitant antibiotics are antibiotics that were taken on or after the first dose of study drug [symbol]
[1] IV route of administration.
[2] Oral route of administration.
ATC = Anatomical Therapeutic Chemical [symbol] IV = intravenous [symbol] PIP/TAZO = piper [symbol] WHO = World Health Organization [symbol]
Source: Post-text Table [symbol] [symbol]
indicates data missing or illegible when filed

### Efficacy and PK Results

### Treatment Failures

Treatment failure was defined as a subject with non-healing or worsening status of their surgical site requiring serious intervention by Week 12. Treatment failure occurred in a lower percentage of subjects who were treated with MBN-101 compared to placebo. Occurrence of treatment failure and the corresponding difference (95% QI comparing treatment with MBN-101 vs placebo was 1 (16.7%) subject m the 0.5 μg/cm² dose group (difference=−26.2 [−72.3, 28.8]), 2 (33.3%) subjects in the 1.5 μg/cm² dose group (difference=−9.5 [−61.0, 45.2]), 1 (16.7%) subject in the 5.0 μg/cm² dose group (difference=−26.2 [−72.3, 28.8]), and 3 (42.9%) subjects in the placebo group.

In total, 3 subjects were considered a treatment failure due to a non-healing or worsening status of their surgical site requiring serious intervention by Week 12: 1 subject (Subject 001-002) in the 0.5 μg/cm² dose group, 1 subject (Subject 001-007) in the 1.5 μg/cm² dose group, and 1 subject (Subject 014-011) in the placebo group. Four subjects were considered a treatment failure due to loss to follow-up or withdrawal by the subject prior to Week 12. 1 subject (Subject 014-003) in the 1.5 μg/cm² dose group, 1 subject (Subject 001-010) in the 5.0 μg/cm² dose group, and 2 subjects (Subject 001-001 and Subject 001-011) in the placebo group. Two subjects required serious intervention by Week 12, but were not considered treatment failures due to an improved or healed status of their surgical site: 1 subject (Subject 006-003) in the 0.5 μg/cm² dose group and 1 subject (Subject 020-001) in the 1.5 μg/cm² dose group.

Treatment failure in subjects with baseline infections resistant to 1 or more antibiotics based on central laboratory microbiological data for the mITT Population was also evaluated. Occurrence of treatment failure and the corresponding difference (95% CI) comparing treatment with MBN-101 vs placebo in subjects with baseline infections resistant to 1 or more antibiotics was 1 out of 4 (25.0%) subjects in the 0.5 μg/cm² dose group (difference=−25.0 [−83.0, 51.0]), 2 out of 5 (40.0%) subjects in the 1.5 μg/cm² dose group (difference=−10.0 [−70.1, 56.1]), 1 out of 4 (25.0%) subjects in the 5.0 μg/cm² dose group (difference=−25.0 [−83.0, 51.0]), and 2 out of 4 (50.0%) subjects in the placebo group.

### Serious Intervention

The 5.0 μg/cm² (highest) dose group was the only treatment group that did not have a subject with a serious intervention during after surgery during the study. Throughout the duration of the study, occurrence of a serious intervention and the corresponding difference (95% CI) comparing treatment with MBN-101 vs placebo was 2 (33.3%) subjects in the 0.5 μg/cm² dose group (difference=19.0 [−37.2, 66.4]), 2 (33.0%) subjects in the 1.5 μg/cm² dose group (difference=19.0 [−37.2, 66.4]), and 1 (14.3%) subject in the placebo group.

### Readmission

The mean time to readmission was 66.0 days for 2 (33.3%) subjects in the 0.5 μg/cm² dose group, 21.0 days for 1 (16.7%) subject in the 1.5 μg/cm² dose group, and 27.0 days for 1 (14.3%) subject in the placebo group. Subjects who did not have any readmission were censored to the last observation date. The majority (≥66.7%) of subjects in each treatment group were censored. Censored subjects comprised 4 (66.7%) subjects in the 0.5 μg/cm² dose group, 5 (83.3%) subjects in the 1.5 μg/cm² dose group, 6 (100.0%) subjects in the 5.0 μg/cm² dose group, and 6 (85.7%) subjects in the placebo group.

### Reoperation

The mean time to reoperation was 72.5 days for 2 (33.3%) subjects in the 0.5 μg/cm² dose group and 29.5 days for 2 (33.3%) subjects in the 1.5 μg/cm² dose group. Subjects who did not have any reoperation were censored to the last observation date. The majority (≥66.7%) of subjects in each treatment group were censored. Censored subjects comprised 4 (66.7%) subjects in the 0.5 μg/cm² dose group, 4 (66.7%) subjects in the 1.5 μg/cm² dose group, 6 (100.0%) subjects in the 5.0 μg/cm² dose group, and 7 (100.0%) subjects in the placebo group.

### Self-Reported Outcomes

Veterans RAND 12-item Health Survey: No trends were observed in the mean PCS (public health domain score) from baseline to Week 6 and Week 12 in the MBN-101 dose groups for the mITT population. There was a mean increase in the MCS (mental health domain score) from baseline to Week 6 and Week 12 in the MBN-101 dose groups; no such trend occurred in the placebo group at Week 12.

There were no meaningful differences between the treatment groups or trends in the mean change of SMFA dysfunction or bother indices from baseline to Week 6 and Week 12 for the mITT Population.

### Microbiological Success

Microbiological success was assessed by clearance of infection, which was defined as eradication of all baseline pathogens, at the time of any subsequent surgical procedure at the index site. Microbiological success was assessed in a small number of subjects due to the requirement that subjects must have had subsequent surgery for microbiological culture to be performed. The majority of subjects did not have a subsequent surgical procedure of the index site to obtain a culture; these subjects were postulated to be a clinical cure. In total, 3 (50.0%) subjects in the 0.5 μg/cm² dose group and 1 (16.7%) subject in the 1.5 μg/cm² dose group were assessed for microbiological success. None of the 4 subjects assessed had microbiological success on Day 2 through Week 12.

Table 30 present microbiological results from the local and central laboratory for subjects with baseline and post-baseline microbiological assessment for the ITT Population. Specimens from superficial swabs, deep swabs, and tissue were obtained on the day of surgery (baseline) from all subjects. Four subjects treated with MBN-101 had additional specimens collected at various time points after baseline surgery.

TABLE 30
Microbiological Results from Local and Central Laboratory for Subjects With Baseline
and Post-Baseline Microbiological Assessment - Intent-to-Treat Population

| | | | | | |
|---|---|---|---|---|---|
| | | | | BisEDT | BisEDT |
| | | | BisEDT | MIC (mg/L) | MIC (mg/L) |
| Dose Group | Source of | | MIC (mg/L) | for Specimen | for Specimen |
| Subject | Specimen | Isolated Pathogen(s) | at Baseline [1] | Collection #2 | Collection #3 |

0.5 μg/cm

[symbol]

| | | | | | |
|---|---|---|---|---|---|
| 001-002 [2] | Deep swab | S. epidermidis | 0.03 | 0.03 | — |
| | Superficial swab | No growth | — | — | — |
| | Tissue | S paste [symbol] ri | 0.06 | — | — |
| | Deep swab | C. [symbol] | — | 0.06 | — |
| | Superficial swab | S. epidermidis | — | 0.03 | — |
| | Superficial swab | C. [symbol] | — | 0.12 | — |
| | Tissue | S. epidermidis | — | 0.06 | — |
| | Tissue | C. [symbol] | — | 0.06 | — |
| | Deep swab | K. pneumo [symbol] ae | — | — | 2 |
| | Superficial swab | K. pneumo [symbol] ae | — | — | 2 |
| | Tissue | K. pneumo [symbol] ae | — | — | 2 |
| 006-002 [3] | Superficial swab | E. cloacae | 2 | — | — |
| | Superficial swab | S. mar [symbol] | 1 | Not tested [4] | — |
| | Superficial swab | [symbol] | 1 | — | — |
| | Superficial swab | S. oralis | 0.5 | — | — |
| | Superficial swab | E. faecalis | 2 | — | — |
| | Superficial swab | S. aure [symbol] MSSA | 0.12 | — | — |
| | Deep swab | S. marc [symbol] | 1 | — | — |
| | Deep swab | K. [symbol] toca | 1 | — | — |
| | Deep swab | E. faecalis | 2 | — | — |
| | Deep swab | S. aure [symbol] MSSA | 0.12 | — | — |
| | Deep swab | S. [symbol] ensis | 0.06 | — | — |
| | Deep swab | C. re [symbol] | 0.06 | — | — |
| | Tissue | K. [symbol] toca | 1 | — | — |
| | Tissue | S. marc [symbol] | 2 | — | — |
| | Tissue | E. faecalis | 2 | — | — |
| | Tissue | S. aure [symbol] MSSA | 0.12 | — | — |
| | Tissue | S. oralis | 0.5 | — | — |
| 006-003 [5] | Superficial swab | S. aure [symbol] MRSA | 0.06 | 0.06 | — |
| | Deep swab | S. aure [symbol] MRSA | — | 0.06 | — |
| | Superficial swab | C. [symbol] | 0.0 [symbol] 99 | — | — |
| | | (formerly P. [symbol] ) | | | |
| | Deep swab | S. epidermidis | 0.0 [symbol] | — | — |
| | Deep swab | S. warn [symbol] | 0.06 | — | — |
| | Tissue | S. aure [symbol] MRSA | 0.06 | 0.06 | — |

1.5 μg/cm

[symbol]

| | | | | | |
|---|---|---|---|---|---|
| 001-007 [6] | Deep swab | S. aure [symbol] MSSA | 0.12 | — | — |
| | Deep swab | F. magna | 0.0599 | — | — |
| | Superficial swab | S. aure [symbol] MSSA | 0.12 | — | — |
| | Superficial swab | S. epidermidis | 0.0149 | — | — |
| | Superficial swab | C. [symbol] mycola [symbol] | 0.12 | — | — |
| | Superficial swab | F. magna | 0.12 | — | — |
| | Superficial swab | P. g [symbol] bachii | 0.0599 | — | — |
| | Tissue | S. aure [symbol] MSSA | 0.12 | — | — |
| | Tissue | F. magna | 0.12 | — | — |
| | Superficial swab | S. lag [symbol] | — | 0.03 | — |
| | Deep swab | S. lag [symbol] | — | 0.03 | — |
| | Tissue | S. lag [symbol] | — | 0.03 | — |
| | Tissue | S. epidermidis | — | 0.03 | — |
| | Tissue | S. haem [symbol] yticus | — | 0.06 | — |

[symbol]

indicates data missing or illegible when filed

For Subject 001-002 in the 0.5 pig/cm² dose group at baseline, S. epidermidis and S. pasteuri were isolated from deep swab and tissue, respectively; no growth of any pathogen was obtained from superficial swab. Approximately 3 months after surgery, S. epidermidis and Corynebacterium striatum were isolated from superficial swab, S. epidermidis and C. striatum were isolated from deep swab, and S. epidermidis and C. striatum were isolated from tissue. At approximately 4.5 months after baseline surgery, only K. pneumoniae was isolated from superficial swab, deep swab, and tissue. The BisEDT MIC values were unchanged for pathogens isolated at more than time point from the same source.

For Subject 006-002 in the 0.5 μg/cm² dose group at baseline, E. cloacae, Serratia marcescens, Klebsiella oxytoca, S. oralis, Enterococcus faecalis, and S. aureus (MSSA) were isolated from superficial swab; S. marcescens, K. oxytoca, E faecalis, S. aureus (MSSA), S. lugdunensis, and C. resistens were isolated from deep swab; and K. oxytoca, S. marcescens, E faecalis, S. aureus (MSSA), and S. oralis were isolated from tissue. At approximately 12 weeks after baseline surgery, only S. marcescens was isolated from superficial swab. The BisEDT MIC value for S. marcescens isolated from superficial swab approximately 12 weeks after surgery was not calculated by the local or central laboratory.

For Subject 006-003 in the 0.5 μg/cm² dose group at baseline, S. aureus (MRSA) and C. acnes were isolated from superficial swab, S. epidermidis and S. warneri were isolated from deep swab, and S. aureus (MRSA) was isolated from tissue. Approximately 2 months after baseline surgery, S. aureus (MRSA) was isolated from superficial swab, deep swab, and tissue. The BisEDT MIC was unchanged for S. aureus (MRSA) isolated from tissue and superficial swab at baseline and approximately 2 months after surgery.

For Subject 001-007 in the 1.5 μg/cm² dose group at baseline, S. aureus (MSSA), S. epidermidis, C. amycolatum, F. magna, and P. gorbachii were isolated from superficial swab; S. aureus (MSSA) and F. magna were isolated from deep swab; and S. aureus (MSSA) and F. magna were isolated from tissue. Approximately 1.5 months after baseline surgery, S. lugdunensis was isolated from superficial swab, deep swab, and tissue; S. epidermidis and S. haemolyticus were isolated from tissue.

### Serologic Markers

Table 31 presents a summary of serological markers by visit for the mITT Population.

There was a mean decrease in CRP values from baseline to Week 6 and Week 12 for all treatment groups and a mean decrease in ESRs from baseline to Week 12 for all treatment groups, indicating a reduction in systemic markers of inflammation.

TABLE 31
Summary of Serologic Markers - Modified Intent-to-Treat Population

| | | | | | |
|---|---|---|---|---|---|
| | MBN-101 | MBN-101 | MBN-101 | | |
| | 0.025 mg/mL | 0.075 mg/mL | 0.25 mg/mL | | |
| Parameter (Unit) | (0.5 μg/cm [symbol] ) | (1.5 μg/cm [symbol] ) | (5.0 μg/cm [symbol] ) | Placebo | Total |
| Visit Statistic | (N = 6) | (N = 6) | (N = 6) | (N = 7) | (N = 25) |

[symbol]

-reactive protein (g/L)
Baseline [1]

| | | | | | |
|---|---|---|---|---|---|
| n [2] | 4 | 3 | 3 | 2 | 12 |
| Mean (SD) | 0.0290 | 0.0173 | 0.0175 | 0.0994 | 0.0349 |
| | (0.0438) | (0.0151) | (0.0252) | (0.0 [symbol] 5) | (0.0502) |

Day 14

| | | | | | |
|---|---|---|---|---|---|
| n [2] | 4 | 5 | 4 | 6 | 19 |
| Mean (SD) | 0.0215 | 0.0316 | 0.0045 | 0.1980 | 0.0763 |
| | (0.0159) | (0.0437) | (0.0024) | (0. [symbol] 734) | (0.21 [symbol] 7) |

Change from baseline on Day 14

| | | | | | |
|---|---|---|---|---|---|
| n [3] | 3 | 2 | 2 | 1 | 8 |
| Mean (SD) | −0.0171 | −0.0039 | 0.001 [symbol] | 0.0107 | −0.00 [symbol] 7 |
| | (0.0305) | (0.0124) | (0.0021) | (NA) | (0.0200) |

Week 6

| | | | | | |
|---|---|---|---|---|---|
| n [2] | 5 | 4 | 5 | 7 | 21 |
| Mean (SD) | 0.1914 | 0.0141 | 0.0073 | 0.0707 | 0.0735 |
| | (0.4002) | (0.0102) | (0.0065) | (0.0932) | (0.1999) |

Change from baseline on Week 6

| | | | | | |
|---|---|---|---|---|---|
| n [3] | 4 | 3 | 3 | 2 | 12 |
| Mean (SD) | −0.0165 | −0.0016 | −0.0094 | −0.0747 | −0.0207 |
| | (0.0432) | (0.0071) | (0.0162) | (0.0780) | (0.0423) |

Week 12

| | | | | | |
|---|---|---|---|---|---|
| n [2] | 6 | 5 | 5 | 3 | 19 |
| Mean (SD) | 0.2733 | 0.0174 | 0.0070 | 0.0105 | 0.0944 |
| | (0.6500) | (0.0173) | (0.0063) | (0.0030) | (0.3647) |

Change from baseline on Week 12

| | | | | | |
|---|---|---|---|---|---|
| n [3] | 4 | 3 | 3 | 2 | 12 |
| Mean (SD) | −0.0209 | −0.0034 | −0.0091 | −0.0884 | −0.0248 |
| | (0.0421) | (0.0062) | (0.0167) | (0.1037) | (0.0495) |

Week 24, EOT/Early Termination

| | | | | | |
|---|---|---|---|---|---|
| n [2] | 4 | 0 | 0 | 1 | 5 |
| Mean (SD) | 0.0364 | NA | NA | 0.0060 | 0.0303 |
| | (0.0631) | (NA) | (NA) | (NA) | (0.0 [symbol] 63) |

Change from baseline on Week 24, EOT/Early Termination

| | | | | | |
|---|---|---|---|---|---|
| n [3] | 2 | 0 | 0 | 0 | 2 |
| Mean (SD) | −0.0465 | NA | NA | NA | −0.0465 |
| | (0.0624) | (NA) | (NA) | (NA) | (0.0624) |

Erythrocyte sedimentation rate (mm/h)
Baseline [1]

| | | | | | |
|---|---|---|---|---|---|
| n [2] | 4 | 3 | 3 | 2 | 12 |
| Mean (SD) | 27.0 | #Z.899;1.0 | 37.0 | 84.0 | 40.0 |
| | (19.41) | (24.02) | (54.58) | (43.84) | (36.91) |

Day 14

| | | | | | |
|---|---|---|---|---|---|
| n [2] | 5 | 5 | 4 | 5 | 19 |
| Mean (SD) | 52.8 | 28.4 | 29.8 | 54.8 | 42.1 |
| | (51.79) | (19.89) | (32.01) | ( [symbol] 0.74) | (35.04) |

Change from baseline on Day 14

| | | | | | |
|---|---|---|---|---|---|
| n [3] | 4 | 2 | 2 | 1 | 9 |
| Mean (SD) | 4.0 | −31.0 | 16.5 | −11.0 | −2.7 |
| | (15.87) | (8.49) | (24.7#Z,899) | (NA) | (22.44) |

Week 6

| | | | | | |
|---|---|---|---|---|---|
| n [2] | 5 | 4 | 5 | 7 | 21 |
| Mean (SD) | 53.4 | 39.3 | 20.6 | 51.9 | 42.4 |
| | ( [symbol] 3.13) | (16.32) | (32.5#Z,899) | (28.40) | (35.22) |

Change from baseline on Week 6

| | | | | | |
|---|---|---|---|---|---|
| n [3] | 4 | 3 | 3 | 2 | 12 |
| Mean (SD) | 6.0 | 8.0 | −9.7 | −30.5 | −3.5 |
| | (15.21) | (11.79) | (11.59) | (10.61) | (18.30) |

Week 12

| | | | | | |
|---|---|---|---|---|---|
| n [2] | 6 | 5 | 4 | 5 | 20 |
| Mean (SD) | 49.7 | 23.2 | 18.0 | 23.0 | 30.1 |
| | (46.37) | (14.86) | (20.93) | (16.57) | (30.28) |

Change from baseline on Week 12

| | | | | | |
|---|---|---|---|---|---|
| n [3] | 4 | [symbol] | 2 | 2 | 11 |
| Mean (SD) | −1.8 | −9.0 | −25.5 | −64.5 | −19.5 |
| | (15.20) | (22.61) | ( [symbol] ) | (61.52) | (35.37) |

Week 24, EOT/Early Termination

| | | | | | |
|---|---|---|---|---|---|
| n [2] | 4 | 0 | 0 | 1 | 5 |
| Mean (SD) | 6 [symbol] .0 | NA | NA | 20.0 | 54.4 |
| | (44.61) | (NA) | (NA) | (NA) | (43.15) |

Change from baseline on Week 24, EOT/Early Termination

| | | | | | |
|---|---|---|---|---|---|
| n [3] | 2 | 0 | 0 | 0 | 2 |
| Mean (SD) | −14.5 | NA | NA | NA | −14.5 |
| | ( [symbol] 1.82) | (NA) | (NA) | (NA) | (31.82) |

Note:
All subjects in Cohort 1 (0.5 μg/cm [symbol] dose group and placebo) required a Week 24 visit per the protocol in effect at the time. No subjects in Cohort 2 (1.5 μg/cm [symbol] dose group and placebo) or Cohort 3 (5.0 μg/cm [symbol] dose group and placebo) required [symbol] Week 24 visit.
[1] Baseline was defined as the [symbol] measurement or assessment prior to the first dose of study drug [symbol]
[2] [symbol] was the number of subjects at the specified visit [symbol]
[3] [symbol] was the number of subjects with both baseline and post-baseline measurements [symbol]
EOT = end of treatment [symbol] NA = not applicable [symbol] SD = standard deviation [symbol]
Source: [symbol] Table 14. [symbol] 1 [symbol]
indicates data missing or illegible when filed

### Pharmacokinetic Evaluation

Measurement of Bi levels in blood was performed as a surrogate for BisEDT using a validated inductively coupled plasma mass spectrometry method. Pharmacokinetic parameter analysis was planned but was not performed due to the barely detectable and sporadic Bi levels in MBN-101-treated subjects, indicating low systemic exposure at the administered dose levels (50.600 mg total dose). A few (3 of 7) of the placebo-treated subjects also had low Bi levels that were higher than those in any MBN-101-treated subjects and that were not attributable to BisEDT. Upon further investigation with clinical site investigators, it was determined that some of these subjects were repeatedly treated with Bi-containing wound dressings and/or Bi-containing emollients that were the probable source of Bi in the placebo subjects. This may also be the source of some of the sporadic low blood levels in some of the BisEDT-treated subjects. Thus, no other PK conclusion could be drawn from these data, noting that the total BisEDT dose(s) administered were low (≤0.600 mg total dose), as was any systemic exposure to BisEDT-derived Bi.

### Efficacy Conclusions

Treatment failure rates at Week 12 were lower in all of the MBN-101 dose groups compared to the placebo group. The rates for treatment failure and the corresponding difference comparing treatment with MBN-101 vs placebo for the mITT Population were 16.7% of subjects in the 0.5 μg/cm² dose group (difference=−26.2 [95% CI: −72.3, 28.8]), 33.3% of subjects in the 1.5 μg/cm² dose group (difference=−9.5 [95% CI: −61.0, 45.2]), 16.7<; o of subjects in the 5.0 μg/cm² dose group (difference=−26.2 [95% CI: −72.3, 28.8]), and 42.9% of subjects in the placebo group.

No serious interventions after surgery occurred in the 5.0 μg/cm² (highest) dose group. Serious interventions after surgery occurred in all other treatment groups, including placebo. The rates for serious intervention during the study and the corresponding difference comparing treatment with MBN-101 vs placebo for the mITT Population were 33.3% of subjects in the 0.5 μg/cm² dose group (difference=19.0 [95% CI: −37.2, 66.4]), 33.3% of subjects in the 5 μg/cm² dose group (difference=19.0 [95% CI: −37.2, 66.4]), and 14.3% of subjects in the placebo group. Within the first 4 weeks after surgery, serious intervention occurred only in 2 (33.3%) subjects in the 1.5 μg/cm² dose group (difference=33.3 [95% CI: −23.3, 77.7]). From Week 4 to Week 8 after surgery, serious intervention occurred only in 1 (16.7%) subject in the 1.5 μg/cm² dose group (difference=2.4 [95% CI: −50.2, 51.1]) and 1 (14.3%) subject in the placebo group. From Week 8 to Week 12, serious intervention occurred only in 2 (33.3%) subjects in the 0.5 μg/cm² dose group (difference=33.3 [95% CI: −23.3, 77.7]).

There were no apparent trends in the time to first serious intervention after surgery, time to readmissions, and time to reoperations for the mITT Population.

No subjects had removal of orthopedic hardware due to a healed bone site during the study for the mITT Population.

Local erythema, induration, drainage, and the degree of healing of the surgical site improved in all of the treatment groups by Week 6 and Week 12 compared to baseline for the mITT Population.

There were no meaningful differences between the treatment groups or trends in the VAS surgical site pain scores from baseline to Week 6 and Week 12 for the mITT Population.

Microbiological success was assessed by clearance of infection, which was defined as eradication of all baseline pathogens, at the time of any subsequent surgical procedure at the index site. Microbiological success was assessed in a small number of subjects due to the requirement that subjects must have had subsequent surgery for microbiological culture to be performed. The majority of subjects did not have a subsequent surgical procedure of the index site to obtain a culture; these subjects were postulated to be a clinical cure. In total, 3 (50.0%) subjects in the 0.5 μg/cm² dose group and 1 (16.7%) subject in the 1.5 μg/cm² dose group were assessed for microbiological success. None of the 4 subjects assessed had microbiological success on Day 2 through Week 12.

### Pharmacokinetic Conclusions

Pharmacokinetic analyses were planned but not performed due to the measured Bi concentrations in blood (used as a surrogate for BisEDT levels) being sporadic and low in MBN-101-treated subjects, indicating low systemic exposure at the administered dose levels (50.600 mg total dose). No other PK conclusions could be drawn from these data.

### Safety Results

### Adverse Events

A TEAE was defined as any adverse event starting after the first dose of study drug or placebo was administered. If the adverse event was present prior to the administration of the first dose of study drug or placebo but increased in severity, it was also considered a TEAE. Coding of TEAEs was based on Medical Dictionary for Regulatory Activities (MedDRA) Version 18.1.

Table 32 summarizes the TEAEs for the Safety Population. In total, 16 (64.0%) subjects had a TEAE: 5 (83.3%) subjects in the 0.5 μg/cm² dose group, 4 (66.7%) subjects in the 1.5 μg/cm² dose group, 1 (16.7%) subject in the 5.0 μg/cm² dose group, and 6 (85.7%) subjects in the placebo group. Two (33.3%) subjects in the 1.5 μg/cm² dose group, 1 (16.7%) subject in the 5.0 μg/cm² dose group, and 3 (42.9%) subjects in the placebo group had TEAEs considered mild in severity. One (16.7%) subject in the 0.5 μg/cm² dose group and 3 (42.9%) subjects in the placebo group had TEAEs considered moderate in severity. Three (50.0%) subjects in the 0.5 μg/cm² dose group and 2 (33.3%) subjects in the 1.5 μg/cm² dose group had TEAEs considered severe. One (16.7%) subject in the 0.5 μg/cm² dose group (Subject 001-002) experienced TEAEs considered potentially life-threatening (acute kidney injury, cardiac failure, diabetic ketoacidosis, myocardial ischemia, and septic shock).

In total, 4 (16.0%) subjects had at least 1 TEAE considered possibly related to study drug (MBN-101 or placebo): 1 (16.7%) subject in the 0.5 μg/cm² dose group, 1 (16.7%) subject in the 1.5 μg/cm2 dose group, 1 (16.7%) subject in the 5.0 μg/cm² dose group, and 1 (14.3%) subject in the placebo group. All TEAEs considered possibly related to study drug were considered mild in severity. The outcomes of TEAEs considered possibly related to study drug were resolved in 1 (16.7%) subject in the 0.5 μg/cm² dose group and unknown in 1 (16.7%) subject in the 5 μg/cm² dose group, 1 (16.7%) subject in the 5.0 μg/cm² dose group, and 1 (14.3%) subject in the placebo group.

TABLE 32
Overview of Adverse Events - Safety Population

| | | | | |
|---|---|---|---|---|
| MBN-101 | MBN-101 | MBN-101 | | |
| 0.025 mg/mL | 0.075 mg/mL | 0.25 mg/mL | | |
| (0.5 μg/cm [symbol] ) | (1.5 μg/cm [symbol] ) | (5.0 μg/cm [symbol] ) | Placebo | Total |
| (N = 6) | (N = 6) | (N = 6) | (N = 7) | (N = 25) |
| Subject Events | Subject Events | Subject Events | Subject Events | Subject Events |
| n (%) # | n (%) # | n (%) # | n (%) # | n (%) # |

TEAEs

| | | | | | |
|---|---|---|---|---|---|
| All TEAEs | 5 (83.3) 43 | 4 (66.7) 8 | 1 (16.7) 4 | 6 (85.7) 22 | 16 (64 0) 77 |
| Drug-related TEAEs | 1 (16.7) 2 | 1 (16.7) 1 | 1 (16.7) 3 | 1 (14.3) 2 | 4 (16.0) 8 |

TEAE by maximum severity [1]
All TEAEs

| | | | | | |
|---|---|---|---|---|---|
| Mild | 0 (0.0) 0 | 2 (33.3) 4 | 1 (16.7) 4 | 3 (4.29) 6 | 6 (24.0) 14 |
| Moderate | 1 (16.7) 2 | 0 (0.0) 0 | 0 (0.0) 0 | 3 (42.9) 8 | 4 (16.0) 10 |
| Severe | 3 (50.0) 4 | 2 (33.3) 4 | 0 (0.0) 0 | 0 (0.0) 0 | [symbol] (20.0) 8 |
| Potentially life threatening | 1 (16.7) 5 | 0 (0.0) 0 | 0 (0.0) 0 | 0 (0.0) 0 | 1 (4.0) 5 |

Drug-related TEAEs

| | | | | | |
|---|---|---|---|---|---|
| Mild | 1 (16.7) 2 | 1 (16.7) 1 | 1 (16.7) 3 | 1 (14.3) 2 | 4 (16.0) 8 |
| Deaths | 0 (0.0) 0 | 0 (0.0) 0 | 0 (0.0) 0 | 0 (0.0) 0 | 0 (0.0) 0 |

SAEs

| | | | | | |
|---|---|---|---|---|---|
| All SAEs | 4 (66.7) 14 | 2 (33.3) 3 | 0 (0.0) 0 | 3 (42.9) 3 | 9 (36.0) 20 |
| Drug-related SAEs | 0 (0.0) 0 | 0 (0.0) 0 | 0 (0.0) 0 | 0 (0.0) 0 | 0 (0.0) 0 |

Discontinuation from study due to adverse events

| | | | | | |
|---|---|---|---|---|---|
| All adverse events | 0 (0.0) 0 | 0 (0.0) 0 | 0 (0.0) 0 | 0 (0.0) 0 | 0 (0.0) 0 |

Percentage was calculated using the number of subjects in the column heading as the denominator [symbol]
# was the number of individual occurrences of events [symbol]
A TEAE was defined as any adverse event [symbol] after the first dose of study drug or placebo was administered. If the adverse event was present prior to the administration of the first dose of study drug or placebo but increased in severity [symbol] also considered a TEAE [symbol]
Drug-related adverse events were defined as possible or probable adverse events [symbol]
[1] Both subjects and events were by maximum severity per subject [symbol]
SAE = serious adverse event [symbol] TEAE = treatment-emergent adverse event [symbol]
Source: Pos [symbol] Table 14. [symbol] .1 [symbol]
indicates data missing or illegible when filed

TEAEs are summarized in Table 33 and those occurring in >20% of subjects detailed by system organ class, preferred term and relationship to study drug are detailed in Table 34. The subject incidence of TEAEs in the 5.0 μg/cm² dose group was lower compared to the placebo group and to the other MBN-101 dose groups. In total, TEAEs occurred in 5 (83.3%) subjects in the 0.5 μg/cm² dose group, 4 (66.7%) subjects in the 1.5 μg/cm² dose group, 1 (16.7%) subject in the 5.0 μg/cm² dose group, and 6 (85.7%) subjects in the placebo group. The most commonly reported TEAEs were blood alkaline phosphatase increased and osteomyelitis, which were reported in 5 subjects (2 subjects in the 0.5 μg/cm² dose group and 1 subject each in the 1.5 μg/cm², 5.0 μg/cm² and placebo group) and 3 subjects (2 in the 0.5 pig/cm² dose group and 1 in the placebo group), respectively. The majority of TEAEs were considered mild or moderate in severity. In total, TEAEs considered severe occurred in 5 subjects (3 subjects in the 0.5 μg/cm² dose group and 2 subjects in the 1.5 μg/cm² dose group). One subject in the 0.5 μg/cm² dose group experienced TEAEs considered potentially life-threatening (acute kidney injury, cardiac failure, diabetic ketoacidosis, myocardial ischemia, and septic shock).

TABLE 33
Overview of Adverse Events - Safety Population

| | | | | |
|---|---|---|---|---|
| MBN-101 | MBN-101 | MBN-101 | | |
| 0.025 mg/mL | 0.075 mg/mL | 0.25 mg/mL | | |
| (0.5 μg/cm²) | (1.5 μg/cm²) | (5.0 μg/cm²) | Placebo | Total |
| (N = 6) | (N = 6) | (N = 6) | (N = 7) | (N = 25) |

TEAEs

| | | | | | |
|---|---|---|---|---|---|
| All TEAEs (#) | 43 | 8 | 4 | 22 | 77 |
| Subjects with TEAES (n) | 5 | 4 | 1 | 6 | 16 |
| All Drug-related TEAES (#) | 2 | 1 | 3 | 2 | 9 |
| Subjects with Drug-related TEAEs (n) | 1 | 1 | 1 | 1 | 4 |

All TEAEs by maximum severity (n, #) [1]

| | | | | | |
|---|---|---|---|---|---|
| Mild | 0, 0 | 2, 4 | 1, 4 | 3, 6 | 6, 14 |
| Moderate | 1, 2 | 0, 0 | 0, 0 | 3, 8 | 4, 10 |
| Severe | 3, 4 | 2, 4 | 0, 0 | 0, 0 | 5, 8 |
| Potentially life threatening | 1, 5 | 0, 0 | 0, 0 | 0, 0 | 1, 5 |

Drug-related TEAEs (n, #)

| | | | | | |
|---|---|---|---|---|---|
| Mild | 1, 2 | 1, 1 | 1, 3 | 1, 2 | 4, 8 |

SAEs (n, #)

| | | | | | |
|---|---|---|---|---|---|
| All SAEs (n, #) | 4, 14 | 2, 3 | 0, 0 | 3, 3 | 9, 20 |
| Drug-related SAEs (n) | 0 | 0 | 0 | 0 | 0 |
| Discontinuation due to AEs (n) | 0 | 0 | 0 | 0 | 0 |
| Deaths (n) | 0 | 0 | 0 | 0 | 0 |

n = total number of subjects with event, # = the number of individual occurrences of events.
Drug-related adverse events were defined as possible or probable adverse events.
[1] Both subjects and events were by maximum severity per subject.

TABLE 34
Summary of Treatment-Emergent Adverse Events (≥20% of Subjects in any Treatment
Group) by System Organ Class, Preferred Term, and Relationship - Safety Population

| | MBN-101 | MBN-101 | MBN-101 | | |
|---|---|---|---|---|---|
| | 0.025 mg/mL | 0.075 mg/mL | 0.25 mg/mL | | |
| System Organ Class | (0.5 μg/cm²) | (1.5 μg/cm²) | (5.0 μg/cm²) | Placebo | Total |
| Preferred Term | (N = 6) | (N = 6) | (N = 6) | (N = 7) | (N = 25) |
| Relationship | n | n | n | n | n |
| Any TEAEs (%) | 5 (83.3) | 4 (66.7) | 1 (16.7) | 6 (85.7) | 16 (64.0) |
| None | 3 | 3 | 0 | 4 | 10 |
| Unlikely | 1 | 0 | 0 | 1 | 2 |
| Possible | 1 | 1 | 1 | 1 | 4 |
| Probable | 0 | 0 | 0 | 0 | 0 |
| Gastrointestinal disorders | 1 (16.7) | 0 (0.0) | 0 (0.0) | 2 (28.6) | 3 (12.0) |
| None | 1 | 0 | 0 | 2 | 3 |
| Unlikely | 0 | 0 | 0 | 0 | 0 |
| Possible | 0 | 0 | 0 | 0 | 0 |
| Probable | 0 | 0 | 0 | 0 | 0 |
| Nausea | 0 | 0 | 0 | 2 | 2 |
| None | 0 | 0 | 0 | 2 | 2 |
| Unlikely | 0 | 0 | 0 | 0 | 0 |
| Possible | 0 | 0 | 0 | 0 | 0 |
| Probable | 0 | 0 | 0 | 0 | 0 |
| Infections and infestations | 2 (33.3) | 1 (16.7) | 0 (0.0) | 4 (57.1) | 7 (28.0) |
| None | 2 | 1 | 0 | 4 | 7 |
| Unlikely | 0 | 0 | 0 | 0 | 0 |
| Posible | 0 | 0 | 0 | 0 | 0 |
| Probable | 0 | 0 | 0 | 0 | 0 |
| Osteomyelitis | 2 | 0 | 0 | 1 | 3 |
| None | 2 | 0 | 0 | 1 | 3 |
| Unlikely | 0 | 0 | 0 | 0 | 0 |
| Possible | 0 | 0 | 0 | 0 | 0 |
| Probable | 0 | 0 | 0 | 0 | 0 |
| Investigations | 3 (50.0) | 1 (16.7) | 1 (16.7) | 3 (42.9) | 8 (32.0) |
| None | 1 | 0 | 0 | 1 | 2 |
| Unlikely | 1 | 0 | 0 | 1 | 2 |
| Possible | 1 | 1 | 1 | 1 | 4 |
| Probable | 0 | 0 | 0 | 0 | 0 |
| Blood alkaline | 2 (33.3) | 1 (16.7) | 1 (16.7) | 1 (14.3) | 5 (20.0) |
| phosphatase increased | | | | | |
| None | 0 | 0 | 0 | 0 | 0 |
| Unlikely | 1 | 0 | 0 | 0 | 1 |
| Possible | 1 | 1 | 1 | 1 | 4 |
| Probable | 0 | 0 | 0 | 0 | 0 |

Coding was based on MedDRA Version 18.1.
If the same TEAE (based on preferred term) was reported for the same subject more than once, the TEAE was counted only once for that preferred term and at the strongest relationship to study drug.

The majority of TEAEs were not considered related to study drug and those considered possibly related to study drug were evenly distributed across MBN-101 treatment groups including placebo occurring in 1 subject each in the 0.5 μg/cm², 1.5 μg/cm², and 5.0 μg/cm² dose groups and the placebo group. All TEAEs considered possibly related to study drug were mild in severity. The outcomes of TEAEs considered possibly related to study drug were resolved in 1 subject in the 0.5 μg/cm² dose group and unknown for the remaining 3 subjects.

Liver function findings of blood alkaline phosphatase≥1.5×ULN were noted for 3 subjects. One subject in the 0.5 μg/cm² dose group had a blood alkaline phosphatase value of 202 U/L at Week 24 but was within the normal limit at all other visits. One subject in the 1.5 μg/cm² dose group and 1 subject in the placebo group had blood alkaline phosphatase values of 291 U/L and 211 U/L, respectively, at Week 12. In addition, 1 subject in the 0.5 μg/cm² dose group had TEAEs of aspartate transaminase increased and blood alkaline phosphatase increased, and 1 subject each in the 5.0 μg/cm² dose group and placebo group had a TEAE of blood alkaline phosphatase and gamma glutamyl transferase increased. Treatment-emergent adverse events for all of these specific subjects were considered mild in severity and possibly related to study drug. No potential Hy's Law cases were identified. No other notable safety signals were identified for chemistry or hematology parameters.

The majority of all vital signs, physical examination findings, and 12-lead ECG findings were within normal limits (data not shown). Subjects with abnormal, clinically significant vital signs or ECG findings reverted to normal values by Week 12 with the exception of 1 subject in the placebo group who experienced abnormal systolic and diastolic blood pressure on Day 3, Day 4, Week 6, and Week 12; this subject did not have a TEAE related to vital signs or 12-lead ECGs (data not shown).

### Serious Adverse Events (SAEs)

In total, 9 (36%) subjects had an SAE. None of the SAEs were considered possibly or probably related to study drug (MBN-101 or placebo). No subjects were discontinued from the study due to an adverse event and no subjects died during the study.

The highest dose group, 5.0 μg/cm², was the only dose group with no SAEs. SAEs occurred in 66.7, 33.3, 0.0% of subjects in 0.5, 1.5, 5.0 μg/cm² MBN-101 and 42.9% of placebo. In the 0.5 μg/cm² dose group, a single subject was reported with an event including several SAEs that were considered potentially life-threatening; these SAEs were acute kidney injury, cardiac failure, diabetic ketoacidosis, myocardial ischemia, and septic shock. In the 0.5 μg/cm² dose group, other SAEs that occurred in 1 subject each were device failure, tibia fracture, wound dehiscence, linear immunoglobulin A disease, drug hypersensitivity, multiple injuries, and road traffic accident; 2 subjects experienced osteomyelitis. In the 1.5 μg/cm² dose group, SAEs that occurred in 1 subject each were bacterial sepsis, wound dehiscence, and hematoma. In the placebo group, SAEs that occurred in 1 subject each were deep vein thrombosis, Clostridium difficile infection, and osteomyelitis.

### Safety Conclusions

In total, TEAEs occurred in 5 (83.3%) subjects in the 0.5 μg/cm² dose group, 4 (66.7%) subjects in the 1.5 μg/cm² dose group, 1 (16.7%) subject in the 5.0 μg/cm² dose group, and 6 (85.7%) subjects in the placebo group. The most commonly reported TEAEs were blood alkaline phosphatase increased and osteomyelitis, which were reported in 5 (20.0%) subjects (2 [33.3%] subjects in the 0.5 μg/cm² dose group, 1 [16.7%,] subject in the 1.5 μg/cm² dose group, 1 [16.7%] subject in the 5.0 μg/cm² dose group, and 1 [14.3%1] subject in the placebo group) and 3 (12.0%) subjects (2 [33.3%] subjects in the 0.5 μg/cm² dose group and 1 [14.3%] subject in the placebo group), respectively. The majority of TEAEs were considered mild or moderate in severity. In total, TEAEs considered severe occurred in 5 (20.0%) subjects (3 [50.0%] subjects in the 0.5 μg/cm² dose group and 2 [33.3%] subjects in the 1.5 μg/cm² dose group). In total, 1 (4.0%) subject in the 0.5 μg/cm² dose group (Subject 001-002) experienced TEAEs considered potentially life-threatening (acute kidney injury, cardiac failure, diabetic ketoacidosis, myocardial ischemia, and septic shock).

The majority of TEAEs were not considered related to study drug (MBN-101 or placebo). In total, TEAEs considered possibly related to study drug occurred in 1 (16.7%) subject each in the 0.5 μg/cm², 1.5 μg/cm², and 5.0 μg/cm² dose groups, and 1 (14.3%) subject in the placebo group. All TEAEs considered possibly related to study drug were mild in severity. The outcomes of SAEs considered possibly related to study dmg were resolved in 1 (16.7%) subject in the 0.5 μg/cm² dose group and unknown in 1 (16.7%) subject in the 1.5 μg/cm² dose group, 1 (16.7%) subject in the 5.0 μg/cm² dose group, and 1 (14.3%) subject in the placebo group.

In total, SAEs occurred in 4 (66.7%) subjects in the 0.5 μg/cm² dose group, 2 (33.3%) subjects in the 1.5 μg/cm² dose group, 0 (0.0%) subjects in the 5.0 μg/cm² dose group, and 3 (42.9%) subjects in the placebo group. In the 0.5 μg/cm² dose group, a single subject (Subject 001-002) was reported with an event including several SAEs that were considered potentially life-threatening; these SAEs were acute kidney injury, cardiac failure, diabetic ketoacidosis, myocardial ischemia, and septic shock. In the 0.5 μg/cm² dose group, other SAEs that occurred in 1 subject each were device failure, tibia fracture, wound dehiscence, linear immunoglobulin A disease, drug hypersensitivity, multiple injuries, and road traffic accident; 2 subjects experienced osteomyelitis. In the 1.5 μg/cm² dose group, SAEs that occurred in 1 subject each were bacterial sepsis, wound dehiscence, and hematoma. In the placebo group, SAEs that occurred in 1 subject each were deep vein thrombosis, C. difficile infection, and osteomyelitis. None of the SAEs were considered possibly or probably related to study drug.

The 5.0 μg/cm² dose group was the only dose group with no SAEs, and subject incidence of TEAEs in the 5.0 μg/cm² dose group was lower compared to the placebo group and to the other MBN-101 dose groups.

No subject had an adverse event that led to the discontinuation from the study.

No subject died during the study.

Liver function findings of blood alkaline phosphatase 21.5×ULN were noted for 3 subjects. One subject in the 0.5 μg/cm² dose group had a blood alkaline phosphatase value of 202 U/L at Week 24 but was within the normal limit at all other visits. One subject in the 1.5 μg/cm² dose group and 1 subject in the placebo group had blood alkaline phosphatase values of 291 U/L and 211 U/L, respectively, at Week 12. In addition, 1 subject in the 0.5 μg/cm² dose group had TEAEs of aspartate transaminase increased and blood alkaline phosphatase increased, and 1 subject each in the 5.0 μg/cm² dose group and placebo group had a TEAE of blood alkaline phosphatase and gamma glutamyl transferase increased. Treatment-emergent adverse events for all subjects were considered mild in severity and possibly related to study drug. No potential Hy's Law cases were identified. No other notable safety signals were identified for chemistry or hematology parameters.

The majority of all vital signs, physical examination findings, and 12-lead ECG findings were within normal limits. Subjects with abnormal, clinically significant vital signs or ECG findings reverted to normal values by Week 12 with the exception of 1 subject (Subject 014-011) in the placebo group who experienced abnormal systolic and diastolic blood pressure on Day 3, Day 4, Week 6, and Week 12. Subject 014-011 did not have a TEAE related to vital signs or 12-lead ECGs.

In conclusion, MBN-101 was safe and well tolerated at exposures as high as 5.0 μg/cm² (0.25 mg/mL) when administered according to the dosing algorithm.

### DISCUSSION

This was a randomized, single-blind, placebo-controlled, multi center study to assess the safety and tolerability of single escalating doses of MBN-101 applied directly to target structures within infected osteosynthesis sites during revision surgery, with or without hardware removal and replacement for subjects diagnosed with an apparent fracture site infection, or to sites of chronic or acute-on-chronic osteomyelitis of the long bone extremities or residual amputated limbs. Subjects were randomized in a 3:1 ratio to receive study drug (MBN-101 or placebo). Subjects were enrolled in 3 consecutive dose cohorts and received 0.025, 0.( )75, or 0.25 mg/mL (w:v) of BisEDT formulated as MBN-101 (equal to 0.5, 1.5, or 5.0 μg/cm² BisEDT, respectively, and up to 8 mL, for a dose of up to 0.2, 0.6, or 2.0 mg BisEDT, respectively) or placebo (diluent). Enrollment of the next cohort did not commence until the DRC reviewed all available safety data on all subjects through Week 6 of the study and approved escalation to the next cohort.

The most common pathogens isolated at baseline from the central microbiologic laboratory were E. cloacae, S. epidermidis, S. aureus (MRSA), S. aureus (MSSA), and F. magna. In total, 13 (68.4%) subjects had polymicrobial infections and 6 (31.6%) subjects had monomicrobial infections at baseline based on central microbiologic laboratory data. Almost half of the subjects with polymicrobial infections (9 [47.4%] subjects) at baseline, and none of the subjects with monomicrobial infections at baseline, were infected with anaerobic pathogens.

Bismuth-1,2-ethanedithiol (BisEDT, the active pharmaceutical ingredient in MBN-101) demonstrated low MICs against Gram-positive and Gram-negative aerobic and anaerobic baseline pathogens, including those with multidrug antibiotic resistance. The BisEDT mean MIC of Gram-positive and Gram-negative aerobes ranged from 0.01 to 4.00 mg/L and 1.00 to 2.00 mg/L, respectively. The BisEDT mean MIC of Gram-positive and Gram-negative anaerobes ranged from 0.06 to 0.25 mg/L and 0.06 mg/L, respectively.

Treatment failure was lowest in the 5.0 μg/cm² dose group (16.7%) and the 0.5 μg/cm² dose group (16.7%) with a difference of −26.2 (95% CI: −72.3, 28.8) compared to placebo. Subjects in the 1.5 μg/cm² dose group also had a lower treatment failure rate compared to placebo.

The 5.0 μg/cm² (highest) dose group was the only treatment group that did not have a subject with a serious intervention after surgery during the study.

Treatment-emergent adverse events occurred in 16 (64.0%) subjects. The most commonly reported TEAEs were blood alkaline phosphatase increased, which were considered possibly related to study drug for 1 subject each in the 0.5 μg/cm², 1.5 μg/cm², and 5.0 μg/cm² dose groups, and 1 subject in the placebo group; and osteomyelitis, none of which were considered related to study drug. All TEAEs considered possibly related to study drug were also considered mild in severity. The outcomes of TEAEs considered possibly related to study drug were resolved in 1 subject in the 0.5 μg/cm² dose group and unknown in 1 subject in the 1.5 μg/cm², 1 subject in the 5.0 μg/cm² dose group, and 1 subject in the placebo group. The lowest number of TEAEs occurred in the 5.0 μg/cm² dose group.

Serious adverse events occurred in 9 (36.0%) subjects. None of the SAEs were considered possibly or probably related to study drug. No subject died in the study, and no subject experienced an adverse event that led to discontinuation from the study.

### Overall Conclusions

MBN-101 was safe and well tolerated at all doses, including at exposures as high as 5.0 μg/cm² (0.25 mg/mL). The 5.0 μg/cm² highest dose group was the only dose group with no SAEs, and subject incidence of TEAEs in the 5.0 μg/cm² dose group was lower compared to the placebo group and to the other MBN-101 dose groups. None of the SAEs that occurred in any treatment group were considered possibly or probably related to study drug.

Bismuth-1,2-ethanedithiol (BisEDT, the active pharmaceutical ingredient in MBN-101) demonstrated low MICs against Gram-positive and Gram-negative aerobic and anaerobic baseline pathogens, including those with multidrug antibiotic resistance.

Treatment failure, the primary measure of efficacy in this study, occurred in a lower percentage of subjects who were treated with MBN-101 compared to placebo, including subjects who had baseline infections resistant to at least 1 antibiotic. In addition, the 5.0 μg/cm2 dose group was the only treatment group that did not have serious interventions after surgery during the study. Results from a self-administered survey suggested that subjects treated with MBN-101 perceived that their mental health had improved after treatment from baseline to Week 12 compared to subjects treated with placebo that did not demonstrate this trend. While several trends suggesting clinical activity were noted, statistical significance was not observed due to the small sample size of the study

According to the study, MBN-101 was well tolerated at all doses studied, and trended towards efficacy in the treatment of contaminated hardware and bone after fracture fixation.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. A method for treating osteomyelitis, the method comprising administering to a subject in need thereof a therapeutically effective amount of a bismuth-thiol (BT) composition that comprises BisEDT suspended therein, wherein the composition is topically applied to an infected bone of said patient.

2. The method of claim 1, wherein the osteomyelitis is the result of a bacterial infection.

3. The method of claim 1, wherein the osteomyelitis is the result of a fungal infection.

4. The method of claim 1, wherein the BT composition comprises a plurality of microparticles, wherein at least 70% of said microparticles have a volumetric mean diameter (VMD) from about 0.6 μm to about 2.5 μm.

5. (canceled)

6. (canceled)

7. The method of claim 1, wherein the BT composition comprises a plurality of microparticles, wherein at least 70% of said microparticles have a volumetric mean diameter (VMD) from about 1 μm to about 2 μm.

8. The method of claim 1, wherein said microparticles have a D90 of less than 2 μm, a D50 of less than or equal to 0.72 μm to 1.3 μm, and/or a D10 of less than or equal to 0.33 μm to 0.70 μm.

9. The method of claim 8, wherein said microparticles have a D90 of less than or equal to 1.6 μm.

10. The method of claim 1, wherein the BT composition comprises BisEDT at a concentration greater than about 0.025 mg/mL or greater, about 1% to about 5% methylcellulose, about 0.05% to about 1.0% polysorbate 80, about 1 to 40 mM sodium chloride, and about 2 to 20 mM sodium phosphate at about pH 7.4.

11. (canceled)

12. (canceled)

13. The method of claim 1, wherein the osteomyelitis results from an infection spread through the bloodstream or from nearby tissue.

14. The method of claim 1, wherein the osteomyelitis results from an infection of an open fracture, skin puncture wound or surgical site.

15. The method of claim 1, wherein the osteomyelitis is the result of an infection obtained from an orthopaedic procedure.

16. The method of claim 15, wherein the orthopaedic procedure involves stabilization of a bone fracture with a metal rod or plate, or a bone replacement surgery.

17. The method of claim 1, wherein the osteomyelitis results from a diabetic foot infection.

18. The method of claim 1, wherein the method comprises at least one of: (i) reducing a biofilm (e.g. bacterial and/or fungal), (ii) impairing growth of a biofilm (e.g. bacterial and/or fungal), (iii) preventing initial formation of the biofilm (e.g. bacterial and/or fungal), and/or (iv) preventing reformation of the biofilm (e.g. bacterial and/or fungal).

19. The method of claim 1, wherein the osteomyelitis is caused by one or more pathogens selected from the group consisting of Corynebacterium jeikeium, Corynebacterium, non speciated, Actinomyces turicensis, Corynebacterium amycolatum, Corynebacterium resistens, Corynebacterium simulans, Dermabacter hominis, Staphylococcus epidermidis, Staphylococcus aureus, MRSA, Staphylococcus aureus, MSSA, Staphylococcus lugdunensis, Enterococcus faecalis, Granulicatella, non speciated, Staphylococcus arlettae, Staphylococcus haemolyticus, Staphylococcus hominis, Staphylococcus pasteuri, Staphylococcus warneri, Streptococcus oralis, Enterobacter cloacae, Serratia marcescens, Escherichia coli, Klebsiella oxytoca, Pseudomonas aeruginosa, Stenotrophomonas maltophilia, Cutibacterium (Propionibacterium) acnes, Finegoldia magna, Anaerococcus murdochii, Anaerococcus, non speciated, Clostridium sphenoides, Peptoniphilus gorbachii, Prevotella bergensis, and Candida parapsilosis.

20. The method of claim 19, wherein the osteomyelitis is caused by a Staphylococcus aureus infection.

21. (canceled)

22. (canceled)

23. The method of claim 1, wherein the subject has undergone an orthopaedic procedure.

24. (canceled)

25. The method of claim 1, wherein the subject has chronic or acute-one-chronic osteomyelitis.

26. The method of claim 1, wherein the subject has diabetes, a weakened immune system, a poor blood supply, or a condition that requires hemodialysis.

27. The method of claim 1, wherein the subject has undergone a debridement procedure.

28. (canceled)

29. The method of claim 1, wherein the BisEDT is applied on the site of infection in a concentration greater than about 0.1 μg/cm².

30.-32. (canceled)

33. The method of claim 1, wherein the composition maintains activity on the site of direct application after one week, one month, or two months.

34. The method of claim 1, wherein the BT composition is administered three times per day, two times per day, once daily, every other day, once every three days, three times per week, once every week, once every other week, once every month, or once every other month.

35. (canceled)

36. The method of claim 1, wherein the subject is administered multiple doses of the BT composition daily or weekly for a length of time ranging from about one week to about 12 weeks.

37. (canceled)

38. The method of claim 1, wherein the BT composition is co-administered with one or more antimicrobial agents.

39. (canceled)

40. A method of preventing a microbial infection and/or osteomyelitis, the method comprising administering to a subject in need thereof a therapeutically effective of a bismuth-thiol (BT) composition comprising BisEDT suspended therein, wherein the composition is applied to a site susceptible to a bone infection.

41.-72. (canceled)

73. A method of treating osteomyelitis, bone infection, or infection near a bone or orthopaedic device inserted in a subject, comprising administering to the subject in need thereof a therapeutically effective amount of a composition comprising a bismuth-thiol (BT) compound, wherein the composition is applied to the infection.

74.-145. (canceled)

146. A method for treating an osteosynthesis-associated infection, comprising intraoperatively administering to a subject in need thereof a therapeutically effective amount of a composition, wherein the composition is a suspension of microparticles having a volumetric mean diameter (VMD) from about 0.4 μm to about 5 μm, and comprising BisEDT at a concentration of 0.025 mg/mL or greater, about 1% to about 5% methylcellulose, about 0.1% to about 1% Tween 80®, about 2 mM to about 20 mM sodium chloride, and about 2 mM to about 20 mM sodium phosphate, and wherein the composition is applied either:
(a) directly to structures within infected osteosynthesis sites during revision surgery with or without hardware removal; or
(b) directly to the immediate soft tissue and bone in subjects with chronic or acute-on-chronic osteomyelitis of the long bone extremities or residual amputated limbs.

147.-185. (canceled)
